(19) United States
(12) Patent Application Publication
(10) Pub. No.: US 2026/0347365 A1
(43) Pub. Date: Nov. 5, 2026

(54) COMPOUND FOR REGULATING AND CONTROLLING 15-PGDH ACTIVITY AND PREPARATION METHOD THEREFOR

(71) Applicant: SCINNOHUB PHARMACEUTICAL CO.,LTD, Chengdu, Sichuan Province (CN)

(72) Inventors: Sen JI, Chengdu, Sichuan Province (CN); Hao WANG, Chengdu, Sichuan Province (CN); Wenqing ZHANG, Chengdu, Sichuan Province (CN); Xiao WANG, Chengdu, Sichuan Province (CN); Jie LIANG, Chengdu, Sichuan Province (CN); Nan LI, Chengdu, Sichuan Province (CN); Jing WANG, Chengdu, Sichuan Province (CN); Yuanqing TANG, Chengdu, Sichuan Province (CN); Xiaodong ZHANG, Chengdu, Sichuan Province (CN); Jun TANG, Chengdu, Sichuan Province (CN)

(21) Appl. No.: 19/484,189

(22) PCT Filed: May 17, 2024
(86) PCT No.: PCT/CN2024/093993

§ 371 Date: Nov. 13, 2025

(30) Foreign Application Priority Data
May 19, 2023 (CN) 202310569546.8

(51) Int. Cl. C07D 487/04; A61K 31/4985; A61K 31/519; C07D 519/00

(52) U.S. Cl. CPC C07D 487/04; A61K 31/4985; A61K 31/519; C07D 519/00

(57) ABSTRACT

The present application relates to a compound of formula (I) for regulating and controlling 15-PGDH activity, a stereoisomer thereof, a tautomer thereof or a mixture thereof, a pharmaceutically acceptable salt thereof, a solvate thereof, or a prodrug thereof, a pharmaceutical composition containing same, and the use thereof for inhibiting 15-PGDH activity.

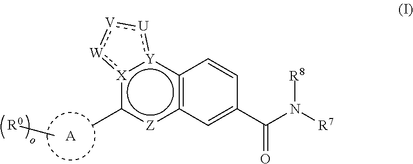

### TECHNICAL FIELD

The present application relates to a compound for regulating the activity of 15-PGDH and a preparation method thereof, specifically relates to a compound that can be used as a medicament for regulating the activity of 15-PGDH, and a pharmaceutically acceptable salt thereof, a composition comprising the compound or the salt thereof, and use thereof in the preparation of a medicament, belonging to the field of pharmaceutical chemistry.

### BACKGROUND

15-hydroxyprostaglandin dehydrogenase (15-PGDH) belongs to an evolutionarily conserved superfamily of short-chain dehydrogenases/reductases (SDRs), and has been designated as SDR36C1 according to the recently approved nomenclature for human enzymes. Based on current research results, the majority of the in vivo activity can be attributed to HPGD gene-encoded type I 15-PGDH. 15-PGDH plays an important role in the inactivation of active prostaglandins (PGD2, PGE1, PGE2, PGF2α, PGI2, etc.), hydroxyeicosatetraenoic acids (HETEs), and inflammation-resolving lipid mediators (RvD1, RvD2, RvE1, MaR1, LXA4, etc.) (hereinafter referred to generally as 15-PGDH substrates) (e.g., by catalyzing an oxidation reaction of hydroxy at position 15 of PGF2α into 15-keto-PGF2α). These 15-PGDH substrates exert their functions through specific receptors on target cells. Among others, prostaglandins, such as PGE1, PGE2, PGF2α, PGI2, etc., are often used to assess the activity of 15-PGDH. For example, the activity of PGDH is assessed by measuring ketone metabolites of hydroxy at position 15 of PGF2α (Journal of Clinical Endocrinology and Metabolism, Vol 84, No. 1, 291-299).

Receptors of 15-PGDH substrates are widely and differentially distributed in vivo, and the diversity of expression distributions, receptor types, and signaling together create a diversity of functions in vivo. For example, PGE1 acts on blood vessels and platelets to prompt an increase in blood flow by vasodilatory effects, and inhibition of platelet aggregation, and is therefore commonly used for treating diseases such as chronic arterial occlusion (thromboangiitis obliterans (TAO) or occlusive arteriosclerosis obliterans (ASO)), skin ulcers; PGF2α has uterine contraction and intraocular pressure lowering effects, and derivatives thereof have been used as therapeutic agents for glaucoma; and PGD2 inhibits inflammation by enhancing the barrier function of the pulmonary blood vessels. In addition, PGE2 has vasodilatory effects, as well as a variety of effects involving blood pressure, pain, bone formation, cell growth, stem cell differentiation, anti-fibrotic and anti-inflammatory effects, etc. PGI2 has an inhibitory effect on platelet activation and a relaxing effect on vascular smooth muscle, and its derivatives are used as therapeutic agents for chronic arterial occlusion and primary pulmonary hypertension. Inflammation-resolving lipid mediators (RvD1, RvD2, RvE1, MaR1, LXA4, etc.) inhibit migration/activation of neutrophils and accelerate apoptosis of neutrophils. In addition, it is indispensable in the process of increasing the phagocytic activity of macrophages to effectively remove apoptotic neutrophils/tissue debris remaining at the site of inflammation. These functions can promote inflammation and maintain homeostasis within the organism. These inflammation-resolving lipid mediators have been reported to show medicinal efficacy in various types of pathology models (e.g., mouse pulmonary inflammation model, colitis model, and liver injury model).

Recent studies indicate that 15-PGDH inhibitors and 15-PGDH agonists may have therapeutic values. A recent study indicates that the expression of 15-PGDH in protection against thrombin-mediated cell death is increased. It is well known that the 15-PGDH causes the inactivation of prostaglandin E2 (PGE2), and the prostaglandin E2 is a downstream product of COX-2 metabolism. Studies have shown that PGE2 is beneficial in a variety of biological processes, such as maintaining hair density and promoting skin wound healing and bone formation.

15-PGDH as an important enzyme in the inactivation of 15-PGDH substrates involves a wide range of in vivo roles, and 15-PGDH inhibitors may be used to prevent or treat diseases associated with 15-PGDH and/or 15-PGDH substrates and/or when there is a need to increase the level of 15-PGDH substrates in the body of a subject.

As mentioned above, some substrates of 15-PGDH have anti-fibrotic, anti-inflammatory, blood flow improvement, growth-promoting, stem cell increase-promoting, smooth muscle contraction/relaxation-promoting, immunosuppression and bone metabolism-affecting effects, etc. Thus, 15-PGDH inhibitors may effectively treat or prevent fibrosis (e.g., pulmonary fibrosis (idiopathic pulmonary fibrosis, etc.), hepatic fibrosis, renal fibrosis, myocardial fibrosis, scleroderma, and myelofibrosis), inflammatory diseases (e.g., chronic obstructive pulmonary disease (COPD), acute lung injuries, sepsis, lung disease and asthma, inflammatory bowel disease (e.g., ulcerative colitis and Crohn's disease), peptic ulcers (e.g., NSAID-induced ulcers), autoinflammatory diseases (e.g., Behçet's disease), vasculitis syndromes, acute liver injury, acute kidney injury, nonalcoholic steatohepatitis (NASH), atopic dermatitis, psoriasis, interstitial cystitis, prostatitis syndrome (e.g., chronic prostatitis/chronic pelvic pain syndrome)), cardiovascular disease (e.g., pulmonary arterial hypertension, angina pectoris, myocardial infarction, heart failure, ischemic heart disease, chronic kidney disease, renal failure, cerebral apoplexy, and peripheral circulatory disorders), trauma (e.g., diabetic ulcers, burns, pressure ulcers, acute mucosal injuries (including Stevens-Johnson syndrome, and mucosal injuries associated with alkylating agents, inhibitors of DNA synthesis, inhibitors of DNA gyrase, and anticancer chemotherapeutic agents, such as antimetabolites, mucosal injuries associated with cellular or humoral immunotherapy, mucosal injuries associated with graft-versus-host disease, such as mucositis or stomatitis), autoimmune diseases (e.g., multiple sclerosis or rheumatoid arthritis), graft-versus-host disease (GVHD), hair growth disorder, osteoporosis, ear diseases (e.g., hearing loss, tinnitus, vertigo, and dysequilibrium), eye diseases (e.g., glaucoma and dry eye), diabetes mellitus, underactive bladder, neutropenia, neurological diseases caused by transplantation of stem cells, bone marrows or organs (e.g., psychoneurosis, neuropathies, neurotoxic diseases, neuropathic pain, and neurodegenerative diseases), and muscle regenerative diseases (e.g., muscular dystrophy, myodystrophy, and muscle injuries); in addition, 15-PGDH inhibitors may also be used to promote cervical ripening.

Compounds and pharmaceutically acceptable salts thereof provided in the present application further meet the requirement of small molecules for inhibiting the activity of 15-PGDH.

### SUMMARY OF THE INVENTION

The present application provides a compound represented by formula (I), a stereoisomer thereof, a tautomer thereof or a mixture form thereof, or a pharmaceutically acceptable salt thereof, or a solvate (e.g., a hydrate) thereof, or a prodrug thereof:

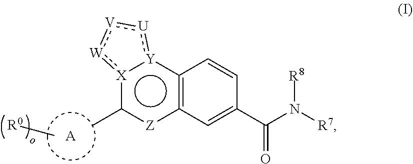

ring A is selected from an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle; o is selected from 0, 1, 2, 3, and 4,
R⁰ is each independently selected from deuterium, tritium, nitro, hydroxyl, mercapto, halogen, cyano, ═O, imino, an amine group, an ester group, an aldehyde group, carboxyl, carbonyl, amido, C₁-C₆ alkyl, C₁-C₆ haloalkyl, C₁-C₆ alkoxy, C₁-C₆ haloalkoxy, 3-12 membered cycloalkyl (i.e., C3-C₁₂ cycloalkyl), and 3-12 membered heterocycloalkyl,
◯ represents an aromatic ring, [symbol] is a single bond or double bond,
Z is selected from N or CH,
X and Y are each independently selected from C or N, and at least one of X, Y, and Z is N,
U, V, and W are each independently selected from N, CR², CR³R⁴, NR⁵, O, and S, wherein the R², R³, R⁴, and R⁵ are each independently selected from hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, 3-12 membered heterocycloalkyl, or the R³ and R⁴, together with the carbon atom to which they are attached, form C—O, C═NR^{W}, or ring B, the R^{W} is selected from hydrogen, deuterium, tritium, C₁-C₆ alkyl, or 3-12 membered cycloalkyl, and the ring B is 3-12 membered cycloalkyl or 3-12 membered heterocycloalkyl,
R⁷ and R⁸ are each independently selected from C₁-C₁₀ chain hydrocarbyl, or R⁷ and R⁸, together with the nitrogen atom to which they are attached, form

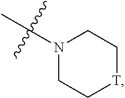

the T is selected from a covalent bond, O, or CR⁹R¹⁰, and the R⁹ and R¹⁰ are each independently selected from hydrogen, deuterium, tritium, nitro, hydroxyl, mercapto, halogen, cyano, —O, imino, an amine group, an ester group, an aldehyde group, carboxyl, amido, C₁-C₆ alkyl, C₁-C₆ haloalkyl, C₁-C₆ alkoxy, and C₁-C₆ haloalkoxy,
wherein R⁰, R², R³, R⁴, R⁵, and ring B are optionally substituted by one or two or more independently selected from deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl, the aromatic heterocycle, the aliphatic heterocycle, the unsaturated aliphatic heterocycle, the fused ring, and the heterocycloalkyl each independently comprise 1-3 heteroatoms which are independently selected from N, O, and S.

In certain embodiments of the present application, the

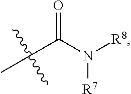

is

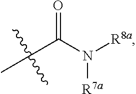

wherein the R^{7a} and R^{8a} are each independently selected from C₁-C₁₀ alkyl; preferably, the R^{7a} and R^{8a} are each independently selected from C₁-C₆ alkyl, preferably, C₁-C₅ alkyl, C₁-C₄ alkyl, or C₁-C₃ alkyl, more preferably, the R^{7a} and R^{8a} are each independently selected from methyl, ethyl, n-propyl, isopropyl, n-butyl, isobutyl, tert-butyl;

In certain embodiments of the present application, the

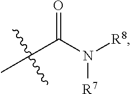

is

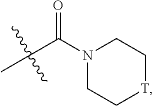

wherein the T is selected from a covalent bond, O, or CR⁹R¹⁰, and the R⁹ and R¹⁰ are each independently selected from hydrogen, deuterium, tritium, nitro, hydroxyl, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, and C₁-C₆ haloalkoxy; preferably, the T is selected from a covalent bond or CR⁹R¹⁰, and the R⁹ and R¹⁰ are each independently selected from hydrogen, deuterium, tritium, nitro, cyano, ═O, —NH₂, —OH, —SH, fluoro, chloro, bromo, iodo, methyl, ethyl, n-propyl, isopropyl, n-butyl, isobutyl, tert-butyl, methoxy, ethoxy, n-propoxy, isopropoxy, trifluoromethyl, trifluoroethyl, trichloromethyl, trichloroethyl, trifluoromethoxy, trifluoroethoxy, trichloromethoxy, and trichloroethoxy; in certain specific embodiments of the present application, the T is selected from a covalent bond, CH₂, CHF, CF₂, CH₂, CHI, CI₂, CHCH₃, and C(CH₃)₂; in certain embodiments of the present application, the T is selected from O;

In certain embodiments of the present application, the

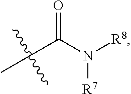

is

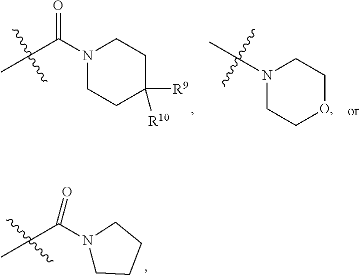

and the R⁹ and R¹⁰ are each independently selected from hydrogen, deuterium, tritium, halogen (fluorine, chlorine, bromine, iodine), C₁-C₃ alkyl, and C₁-C₃ haloalkyl, preferably, the R⁹ and R¹⁰ are each independently selected from hydrogen, deuterium, tritium, and halogen (fluorine, chlorine, bromine, iodine);

In certain embodiments of the present application, the Y and Z are selected from N, and the X is selected from C;

In certain embodiments of the present application, the X and Z are selected from N, and the Y is selected from C;

In certain embodiments of the present application, the Z is selected from N, and the X and Y are selected from C;

In certain embodiments of the present application, the Z, X, and Y are all selected from N;

In certain embodiments of the present application, the Z is selected from CH, the X is selected from N, and Y is selected from C;

In certain embodiments of the present application, the Z is selected from CH, the X is selected from C, and Y is selected from N;

In certain embodiments of the present application, the Z is selected from CH, and both the X and Y are selected from N;

In certain embodiments of the present application, the Z is N, and one of the X and Y is N and the other is C;

In certain embodiments of the present application, the

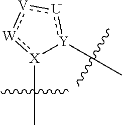

is selected from the following groups:

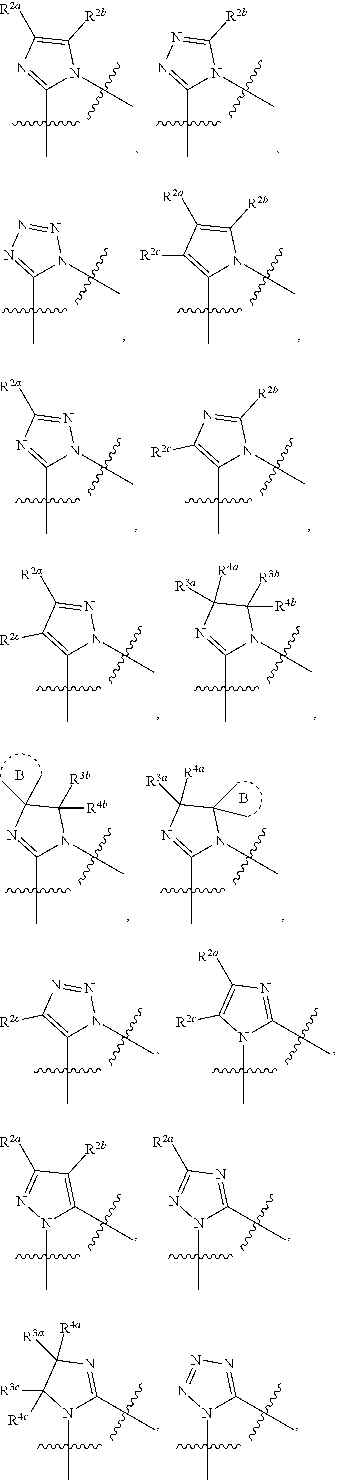

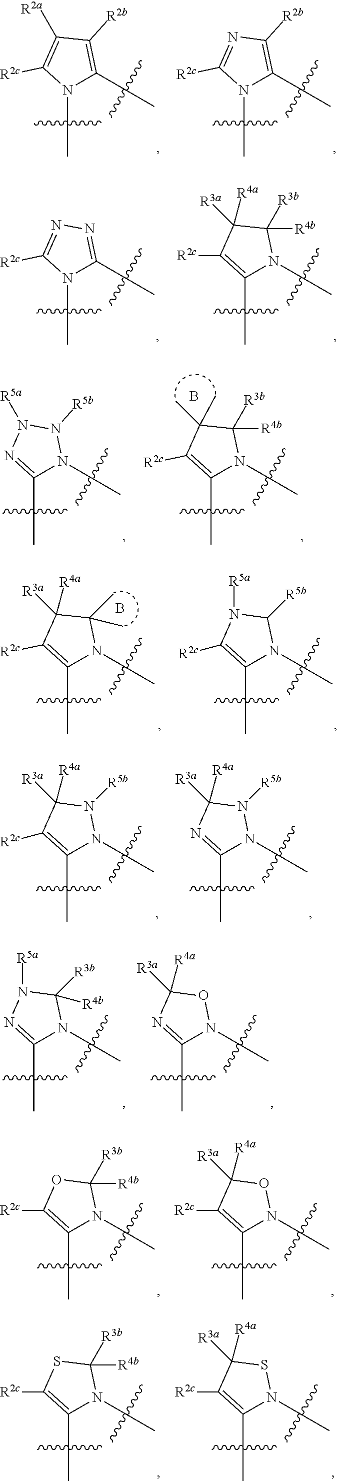

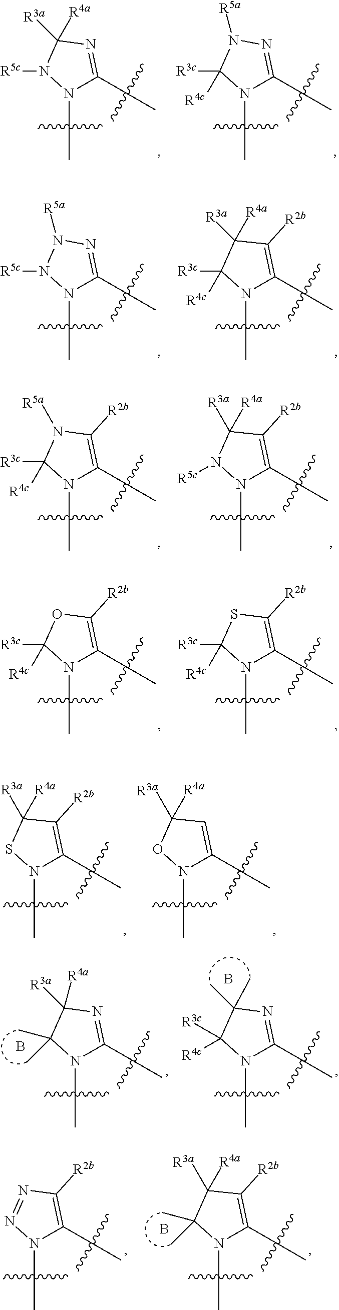

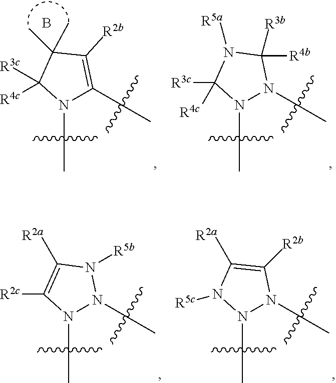

or in certain embodiments of the present application, the

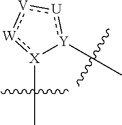

is selected from the following groups:

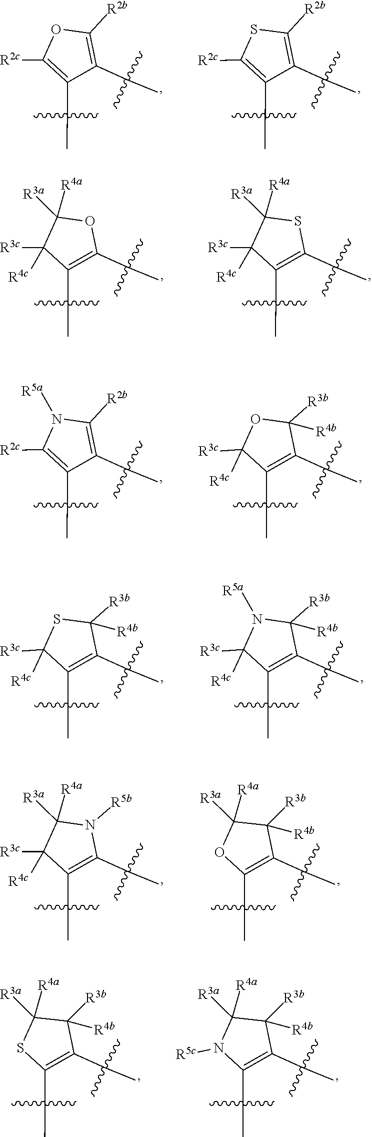

wherein the ring B is 3-12 membered cycloalkyl or 3-12 membered heterocycloalkyl, and the R^{2a}, R^{2b}, R^{2c}, R^{3a}, R^{3b}, R^{3c}, R^{4a}, R^{4b}, R^{4c}, R^{5a}, R^{5b}, and R^{5c} are each independently selected from hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl, or the R^{3a} and R^{4a}, the R^{3b} and R^{4b}, and the R^{3c} and R^{4c}, respectively, together form ═O or imino, and the ring B, R^{2a}, R^{2b}, R^{2c}, R^{3a}, R^{3b}, R^{3c}, R^{4a}, R^{4b}, R^{4c}, R^{5a}, R^{5b}, and R^{5c} are optionally substituted by one or two or more independently selected from deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl;

Preferably, in certain specific embodiments of the present application, the

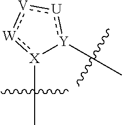

is selected from

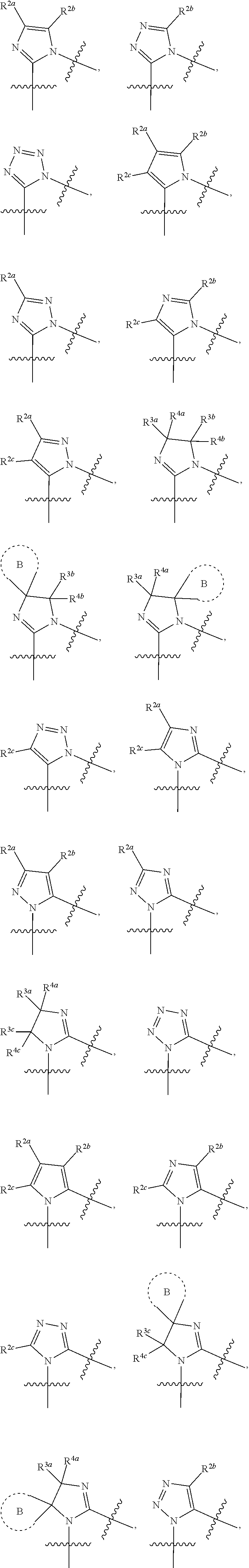

wherein the ring B, R^{2a}, R^{2b}, R^{2c}, R^{3a}, R^{3b}, R^{3c}, R^{4a}, R^{4b}, and R^{4c} are the same as defined above; Preferably, in certain specific embodiments of the present application, the ring B is selected from cyclopropyl, cyclobutyl, cyclopentyl,

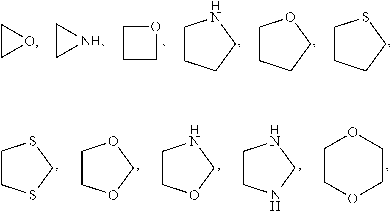

and the R^{2a}, R^{2b}, R^{2c}, R^{3a}, R^{3b}, R^{3c}, R^{4a}, R^{4b}, R^{4c}, R^{5a}, R^{5b}, and R^{5c} are each independently selected from hydrogen, deuterium, tritium, nitro, hydroxyl, mercapto, fluoro, chloro, bromo, iodo, cyano, —NH₂, carboxyl, —CONH₂, —NHCOOH, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl, or the R^{3a} and R^{4a}, the R^{3b} and R^{4b}, the R^{3c} and R^{4c}, respectively, together form ═O, ═NH, wherein the ring B, R^{2a}, R^{2b}, R^{2c}, R^{3a}, R^{3b}, R^{3c}, R^{4a}, R^{4b}, R^{4c}, R^{5a}, R^{5b}, and R^{5c} are optionally substituted by one or two or more independently selected from deuterium, tritium, nitro, hydroxyl, an aldehyde group, an amine group, imino, halogen, cyano, an ester group, carboxyl, amido, —O, C₁-C₆ alkyl, C₁-C₆ alkoxy, 3-8 membered cycloalkyl, and 3-8 membered heterocycloalkyl;

In certain embodiments of the present application,

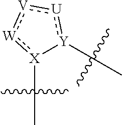

is selected from

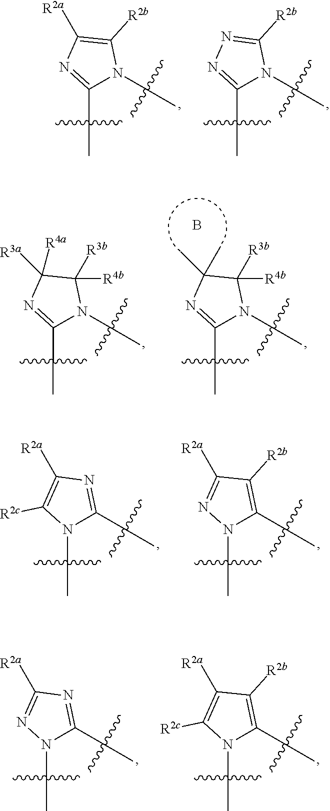

the ring B is selected from cyclopropyl, cyclobutyl, cyclopentyl,

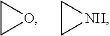

and the R^{2a}, R^{2b}, R^{2c}, R^{3a}, R^{3b}, R^{4a}, and R^{4b} are each independently selected from hydrogen, deuterium, tritium, hydroxyl, cyano, —NH₂, C₁-C₆ alkyl (preferably C₁-C₅ alkyl, C₁-C₄ alkyl, or C₁-C₃ alkyl, such as methyl, ethyl, n-propyl, isopropyl, n-butyl, isobutyl, tert-butyl), C₁-C₆ haloalkyl (preferably C₁-C₅ haloalkyl, C₁-C₄ haloalkyl, or C₁-C₃ haloalkyl, such as trifluoromethyl, trifluoroethyl, trichloromethyl, trichloroethyl), cyclopropyl, cyclobutyl, and cyclopentyl;

In certain embodiments of the present application, the R⁰ is each independently selected from deuterium, tritium, hydroxyl, halogen (fluorine, chlorine, bromine, iodine), cyano, ═O, imino, an amine group, an ester group (such as —C(O)OR, where R is C₁-C₆ alkyl, C₁-C₅ alkyl, C₁-C₄ alkyl, or C₁-C₃ alkyl), an aldehyde group, carboxyl, carbonyl, amido, C₁-C₆ alkyl (preferably C₁-C₈alkyl, C₁-C₄ alkyl, or C₁-C₃ alkyl), C₁-C₆ haloalkyl, C₁-C₆ alkoxy, C₁-C₆ haloalkoxy, and 4-10 membered heterocycloalkyl comprising 1-3 heteroatoms selected from N or O or S; preferably, the R⁰ is each independently selected from deuterium, tritium, hydroxyl, halogen (fluorine, chlorine, bromine, iodine), cyano, ═O, imino, an amine group, an ester group (such as —C(O)OR, R is C₁-C₆ alkyl, C₁-C₅ alkyl, C₁-C₄ alkyl, or C₁-C₃ alkyl), an aldehyde group, carboxyl, carbonyl, amido, C₁-C₆ alkyl (preferably C₁-C₅ alkyl, C₁-C₄ alkyl, or C₁-C₃ alkyl), C₁-C₆ haloalkyl, C₁-C₆ alkoxy, C₁-C₆ haloalkoxy, and 5-8 membered (such as 5-7 membered or 5-6 membered) heterocycloalkyl comprising 1-3 heteroatoms selected from N or O or S; In certain embodiments of the present application, the o is 0, 1, 2, or 3, such as 0, 1, or 2; In certain embodiments of the present application, the ring A is selected from a 6-10 membered aromatic ring (i.e., a C₆-C₁₀ aromatic ring), a 5-10 membered aromatic heterocycle, a 3-8 membered unsaturated aliphatic heterocycle, a 7-14 membered fused ring consisting of an aromatic ring and an unsaturated aliphatic heterocycle, and a 7-14 membered fused ring consisting of an aromatic heterocycle and an unsaturated aliphatic heterocycle; preferably, the aromatic ring and the aromatic heterocycle are monocyclic or bicyclic, the unsaturated aliphatic heterocycle is monocyclic, and the aromatic heterocycle and the unsaturated aliphatic heterocycle each independently comprise 1-3 heteroatoms, which are independently selected from N, O, S; more preferably, the ring A is selected from benzene ring, naphthalene ring, a 5-10 membered aromatic heterocycle comprising 1-3 heteroatoms, a 6 membered unsaturated aliphatic heterocycle comprising 1-3 heteroatoms (preferably 1-2 heteroatoms), or an 8-14 membered fused ring consisting of benzene ring and an unsaturated aliphatic heterocycle comprising 1-3 heteroatoms (preferably 1-2 heteroatoms), which are independently selected from N, O, S. In some embodiments, the ring A is selected from benzene ring, naphthalene ring, a 5-10 membered aromatic heterocycle comprising 1-3 heteroatoms, or a 9-12 membered (such as a 9-10 membered) fused ring consisting of benzene ring and an unsaturated aliphatic heterocycle comprising 1-3 heteroatoms (preferably 1-2 heteroatoms), which are independently selected from N, O, S.

The present application further provides a compound represented by formula (II), a stereoisomer thereof, a tautomer thereof or a mixture form thereof, or a pharmaceutically acceptable salt thereof, or a solvate (e.g., a hydrate) thereof, or a prodrug thereof:

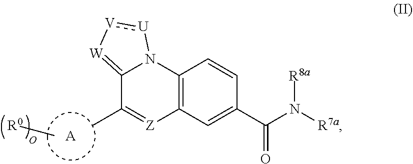

ring A is selected from an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle; Z is selected from N or CH; R^{7a} and R^{8a} are each independently selected from C₁-C₆ alkyl; W is independently selected from N or CR², U and V are each independently selected from N, CR², CR³R⁴, NR⁵, wherein R⁰, o, R², R³, R⁴, R⁵, and [symbol] are the same as defined above; further, the R^{7a}, R^{8a} are each independently selected from methyl, ethyl, n-propyl, isopropyl, n-butyl, isobutyl, and tert-butyl, preferably selected from ethyl.

In certain embodiments of the present application, the

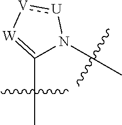

is selected from

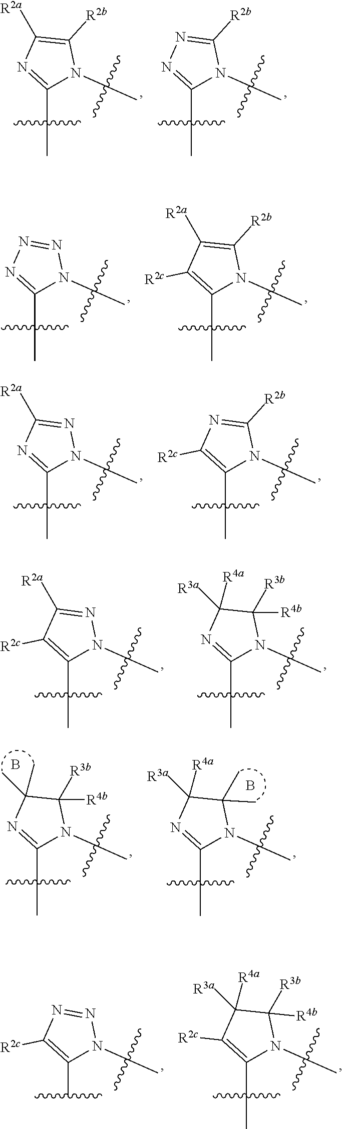

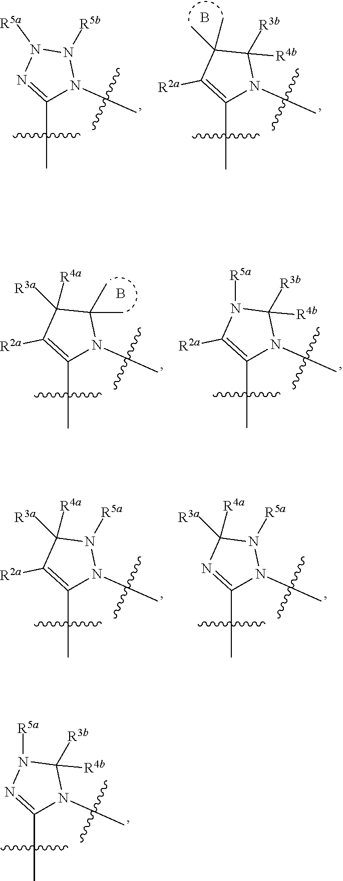

wherein the ring B is 3-12 membered cycloalkyl or 3-12 membered heterocycloalkyl, and the R^{2a}, R^{2b}, R^{2c}, R^{3a}, R^{3b}, R^{4a}, R^{4b}, R^{5a}, and R^{5b} are each independently selected from hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl, or the R^{3a} and R^{4a}, the R^{3b} and R^{4b}, respectively, together form ═O or imino, the ring B, R^{2a}, R^{2b}, R^{2c}, R^{3a}, R^{3b}, R^{4a}, R^{4b}, R^{5a}, and R^{5b} are optionally substituted by one or two or more independently selected from deuterium, tritium, nitro, hydroxyl, an aldehyde group, an amine group, imino, halogen, cyano, an ester group, carboxyl, amido, O, C₁-C₆ alkyl, C₁-C₆ alkoxy, 3-8 membered cycloalkyl, and 3-8 membered heterocycloalkyl;

Preferably, in certain specific embodiments of the present application, the

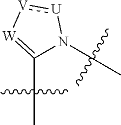

is selected from

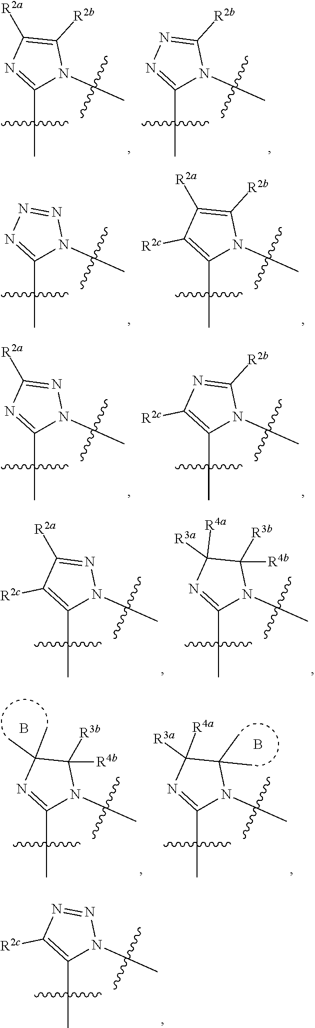

more preferably selected from

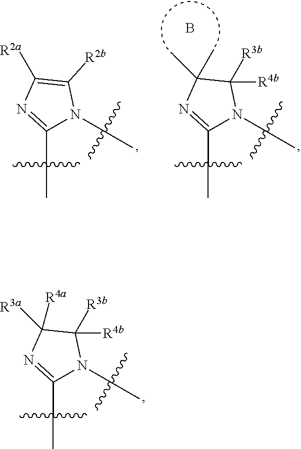

wherein the ring B, R^{2a}, R^{2b}, R^{2c}, R^{3a}, R^{3b}, R^{4a}, and R^{4b} are the same as defined above; preferably, in certain specific embodiments of the present application, the ring B is selected from cyclopropyl, cyclobutyl, cyclopentyl,

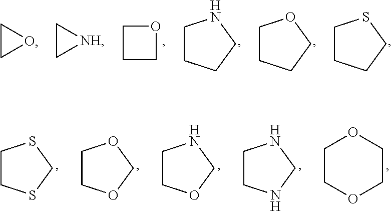

and the R^{2a}, R^{2b}, R^{2c}, R^{3a}, R^{3b}, R^{4a}, R^{4b}, R^{5a}, and R^{5b} are each independently selected from hydrogen, deuterium, tritium, nitro, hydroxyl, mercapto, fluoro, chloro, bromo, iodo, cyano, —NH₂, carboxyl, —CONH₂, —NHCOOH, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl, or the R^{3a} and R^{4a}, the R^{3b} and R^{4b}, respectively, together form ═O, ═NH, wherein the ring B, R^{2a}, R^{2b}, R^{2c}, R^{3a}, R^{3b}, R^{4a}, R^{4b}, R^{5a}, and R^{5b} are optionally substituted by one or two or more independently selected from deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl.

In certain embodiments of the present application, the ring A is selected from a 6-10 membered aromatic ring, a 5-10 membered aromatic heterocycle, a 3-8 membered unsaturated aliphatic heterocycle, a 7-14 membered fused ring consisting of an aromatic ring and an unsaturated aliphatic heterocycle, and a 7-14 membered fused ring consisting of an aromatic heterocycle and an unsaturated aliphatic heterocycle; preferably, the aromatic ring and the aromatic heterocycle are monocyclic or bicyclic, the unsaturated aliphatic heterocycle is monocyclic, and the aromatic heterocycle and the unsaturated aliphatic heterocycle each independently comprise 1-3 heteroatoms, which are independently selected from N, O, S; more preferably, the ring A is selected from benzene ring, naphthalene ring, a 5-10 membered aromatic heterocycle comprising 1-3 heteroatoms, a 6 membered unsaturated aliphatic heterocycle comprising 1-3 heteroatoms (preferably 1-2 heteroatoms), or an 8-14 membered fused ring consisting of benzene ring and an unsaturated aliphatic heterocycle comprising 1-3 heteroatoms (preferably 1-2 heteroatoms), which are independently selected from N, O, S.

In certain specific embodiments of the present application, the compound represented by formula (II) provided in the present application has the structure represented by formula (II-a):

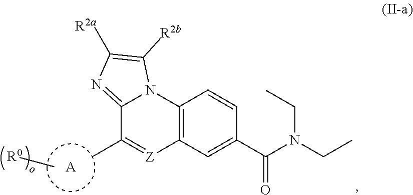

wherein ring A, o, R⁰, Z, R^{2a}, and R^{2b} are the same as defined above.

The present application further provides a compound represented by formula (III), a stereoisomer thereof, a tautomer thereof or a mixture form thereof, or a pharmaceutically acceptable salt thereof, or a solvate (e.g., a hydrate) thereof, or a prodrug thereof:

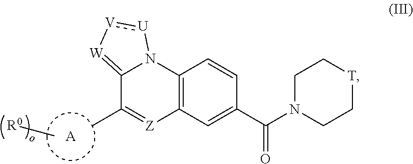

ring A is selected from an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle; Z is selected from N or CH; T is selected from a covalent bond, O, or CR⁹R¹⁰, and the R⁹ and R¹⁰ are each independently selected from hydrogen, deuterium, tritium, nitro, hydroxyl, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, and C₁-C₆ haloalkoxy; W is independently selected from N or CR², and U and V are each independently selected from N, CR², CR³R⁴, and NR⁵, wherein R⁰, o, R², R³, R⁴, R⁵, and [symbol] are the same as defined above; further, the T is selected from a covalent bond, O, or CR⁹R¹⁰, and the R⁹ and R¹⁰ are each independently selected from hydrogen, deuterium, tritium, nitro, cyano, —O, —NH₂, —OH, —SH, fluoro, chloro, bromo, iodo, methyl, ethyl, n-propyl, isopropyl, n-butyl, isobutyl, tert-butyl, methoxy, ethoxy, n-propoxy, isopropoxy, trifluoromethyl, trifluoroethyl, trichloromethyl, and trichloroethyl; preferably, the T is selected from a covalent bond, CH₂, CHF, CF₂, O; more preferably, the T is selected from CH₂, CHF, CF₂.

In certain embodiments of the present application, the

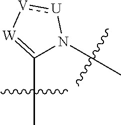

is selected from

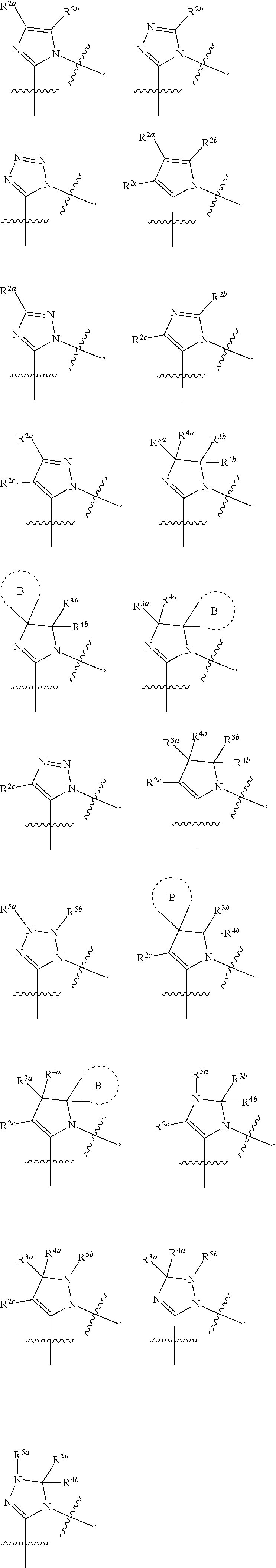

wherein the ring B is 3-12 membered cycloalkyl or 3-12 membered heterocycloalkyl, and the R^{2a}, R^{2b}, R^{2c}, R^{3a}, R^{3b}, R^{4a}, R^{4b}, R^{5a}, and R^{5b} are each independently selected from hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, a 3-12 membered heterocycloalkyl, or the R^{3a} and R^{4a}, the R^{3b} and R^{4b}, respectively, together form-O or imino, and the ring B, R^{2a}, R^{2b}, R^{2c}, R^{3a}, R^{3b}, R^{4a}, R^{4b}, R^{5a}, and R^{5b} are optionally substituted by one or two or more independently selected from deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl; preferably, in certain specific embodiments of the present application, the

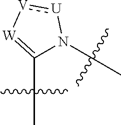

is selected from

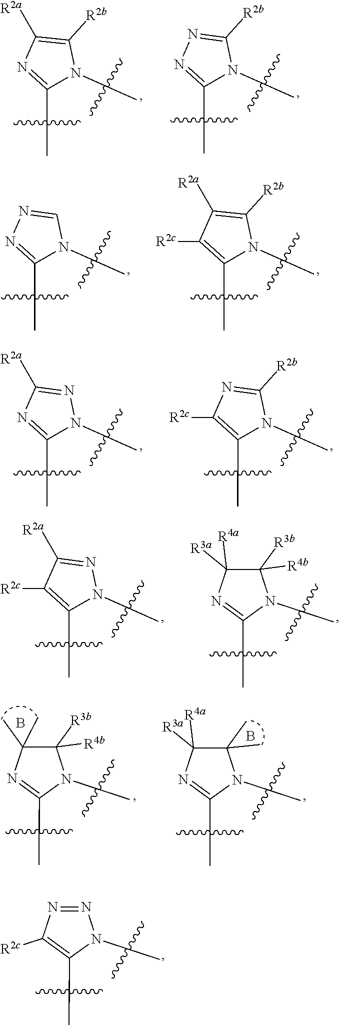

more preferably selected from

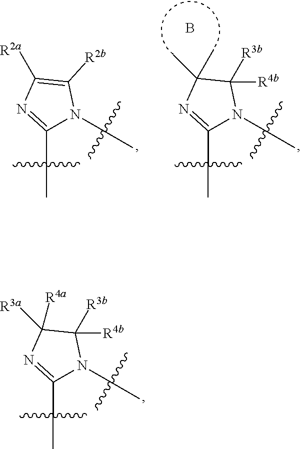

wherein the ring B, R^{2a}, R^{2b}, R^{2c}, R^{3a}, R^{3b}, R^{4a}, and R^{4b} are the same as defined above; preferably, in certain specific embodiments of the present application, the ring B is selected from cyclopropyl, cyclobutyl, cyclopentyl,

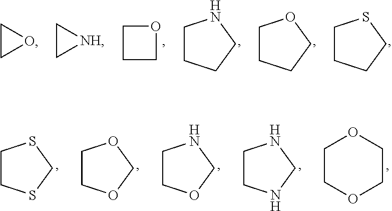

and the R^{2a}, R^{2b}, R^{2c}, R^{3a}, R^{3b}, R^{4a}, R^{4b}, R^{5a}, and R^{5b} are each independently selected from hydrogen, deuterium, tritium, nitro, hydroxyl, mercapto, fluoro, chloro, bromo, iodo, cyano, —NH₂, carboxyl, —CONH₂, —NHCOOH, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl, or the R^{3a} and R^{4a}, the R^{3b} and R^{4b}, respectively, together form-O, ═NH, wherein the ring B, R^{2a}, R^{2b}, R^{2c}, R^{3a}, R^{3b}, R^{4a}, R^{4b}, R^{5a}, and R^{5b} are optionally substituted by one or two or more independently selected from deuterium, tritium, nitro, hydroxyl, an aldehyde group, an amine group, imino, halogen, cyano, an ester group, carboxyl, amido, O, C₁-C₆ alkyl, C₁-C₆ alkoxy, 3-8 membered cycloalkyl, and 3-8 membered heterocycloalkyl.

In certain embodiments of the present application, the ring A is selected from a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle; preferably, the aromatic ring and the aromatic heterocycle are monocyclic or bicyclic, the unsaturated aliphatic heterocycle is monocyclic, and the aromatic heterocycle and the unsaturated aliphatic heterocycle each independently comprise 1-3 heteroatoms, which are independently selected from N, O, S; more preferably, the comprising 1-3 heteroatoms, a 6 membered unsaturated aliphatic heterocycle comprising 1-3 heteroatoms (preferably 1-2 heteroatoms), or an 8-14 membered, preferably an 8-12 membered fused ring consisting of benzene ring and an unsaturated aliphatic heterocycle comprising 1-3 heteroatoms (preferably 1-2 heteroatoms), which are independently selected from N, O, S.

In certain embodiments of the present application, the compound represented by formula (III) provided in the present application has any one of the structures represented by formulas (III-a) to (III-i):

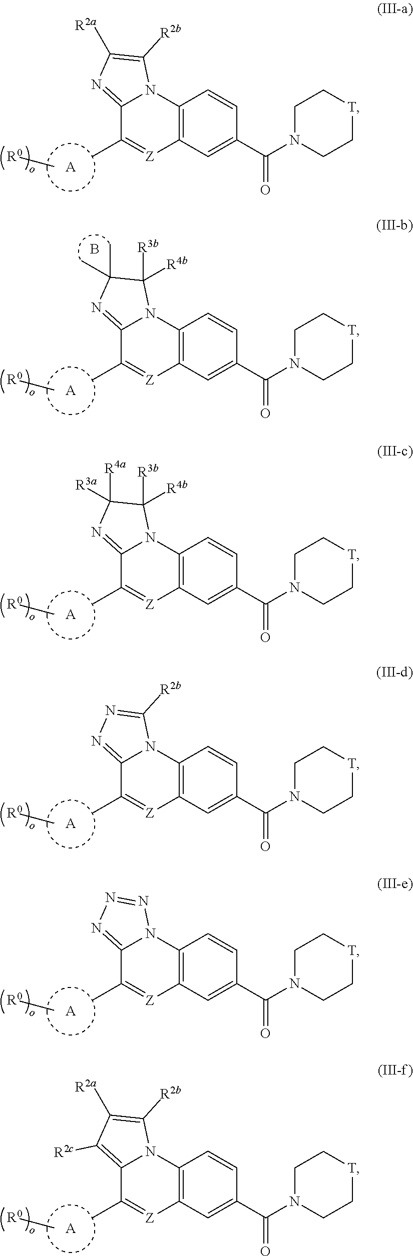

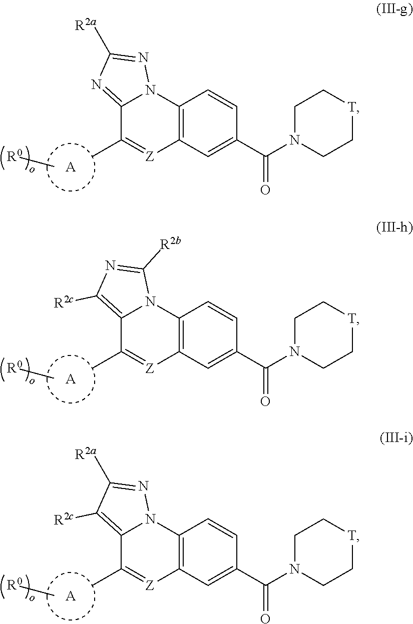

wherein ring A, o, R⁰, Z, R^{2a}, R^{2b}, R^{2c}, R^{3a}, R^{3b}, R^{4a}, and R^{4b} are the same as defined above; ring B is selected from cyclopropyl, cyclobutyl, cyclopentyl,

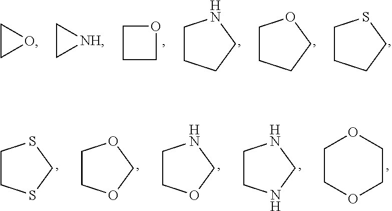

preferably selected from cyclopropyl, cyclobutyl, and cyclopentyl; T is selected from a covalent bond, O, CH₂, CHF, CF₂, preferably selected from CH₂, CHF, and CF₂.

The present application further provides a compound represented by formula (IV), a stereoisomer thereof, a tautomer thereof or a mixture form thereof, or a pharmaceutically acceptable salt thereof, or a solvate (e.g., a hydrate) thereof, or a prodrug thereof:

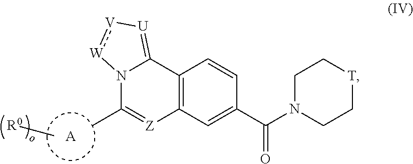

ring A is selected from an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle; Z is selected from N or CH; T is selected from a covalent bond, O, or CR⁹R¹⁰, and the R⁹ and R¹⁰ are each independently selected from hydrogen, deuterium, tritium, nitro, hydroxyl, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, and C₁-C₆ haloalkoxy; U is independently selected from N or CR², and V and W are each independently selected from N, CR², CR³R⁴, and NR⁵; wherein R⁰, o, R², R³, R⁴, R⁵ and [symbol] are the same as defined above; further, the T is selected from a covalent bond, O, or CR⁹R¹⁰, and the R⁹ and R¹⁰ are each independently selected from hydrogen, deuterium, tritium, nitro, cyano, —O, —NH₂, —OH, —SH, fluoro, chloro, bromo, iodo, methyl, ethyl, n-propyl, isopropyl, n-butyl, isobutyl, tert-butyl, methoxy, ethoxy, n-propoxy, isopropoxy, trifluoromethyl, trifluoroethyl, trichloromethyl, and trichloroethyl; preferably, the T is selected from a covalent bond, CH₂, CHF, CF₂, O; more preferably, the T is selected from CH₂, CHF, CF₂.

In certain embodiments of the present application, the

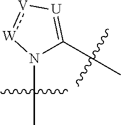

is selected from

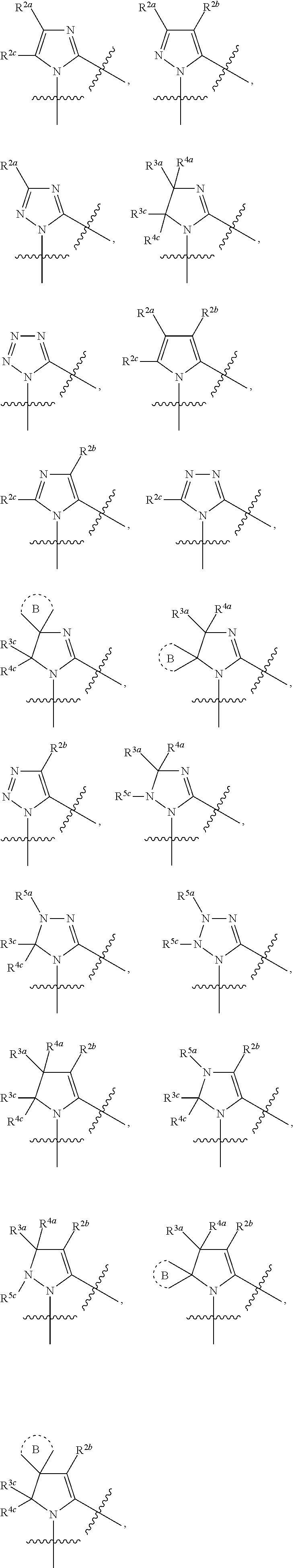

wherein the ring B is 3-12 membered cycloalkyl or 3-12 membered heterocycloalkyl, and the R^{2a}, R^{2b}, R^{2c}, R^{3a}, R^{3c}, R^{4a}, R^{4c}, R^{5a}, and R^{5c} are each independently selected from hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl, or the R^{3a} and R^{4a}, the R^{3c} and R^{4c}, respectively, together form ═O or imino, and the ring B, R^{2a}, R^{2b}, R^{2c}, R^{3a}, R^{3c}, R^{4a}, R^{4c}, R^{5a}, and R^{5c} are optionally substituted by one or two or more independently selected from deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl; preferably, in certain specific embodiments of the present application, the

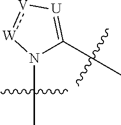

is selected from

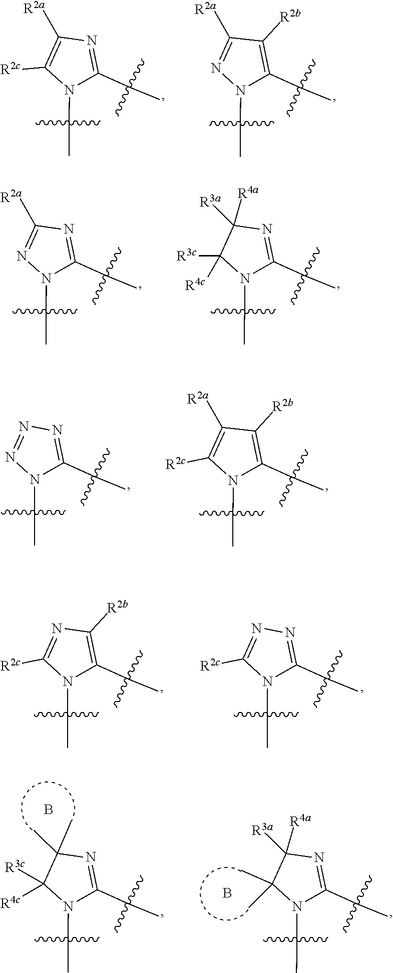

and more preferably selected from

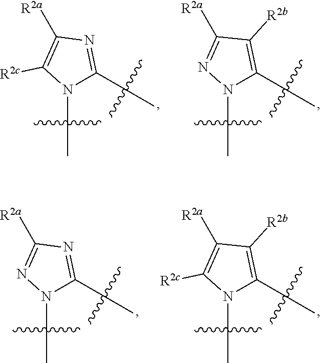

wherein the ring B, R^{2a}, R^{2b}, R^{2c}, R^{3a}, R^{3c}, R^{4a}, and R^{4c} are the same as defined above. Preferably, in certain specific embodiments of the present application, the ring B is selected from cyclopropyl, cyclobutyl, cyclopentyl,

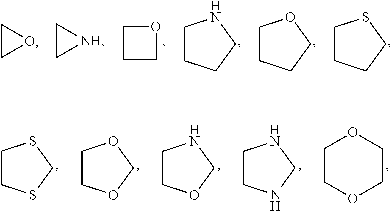

and the R^{2a}, R^{2b}, R^{2c}, R^{3a}, R^{3c}, R^{4a}, R^{4c}, R^{5a}, and R^{5c} are each independently selected from hydrogen, deuterium, tritium, nitro, hydroxyl, mercapto, fluoro, chloro, bromo, iodo, cyano, —NH₂, carboxyl, —CONH₂, —NHCOOH, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl, or the R^{3a} and R^{4a}, the R^{3c} and R^{4c}, respectively, together form ═O, —NH, wherein the ring B, R^{2a}, R^{2b}, R^{2c}, R^{3a}, R^{3b}, R^{4a}, R^{4b}, R^{5a}, and R^{5b} are optionally substituted by one or two or more independently selected from deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl.

In certain embodiments of the present application, the ring A is selected from a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle; preferably, the aromatic ring and the aromatic heterocycle are monocyclic or bicyclic, the unsaturated aliphatic heterocycle is monocyclic, and the aromatic heterocycle and the unsaturated aliphatic heterocycle each independently comprise 1-3 heteroatoms, which are independently selected from N, O, S; more preferably, the ring A is selected from benzene ring, naphthalene ring, a 5-10 membered aromatic heterocycle comprising 1-3 heteroatoms, a 6 membered unsaturated aliphatic heterocycle comprising 1-3 heteroatoms (preferably 1-2 heteroatoms), or an 8-14 membered, preferably an 8-12 membered fused ring consisting of benzene ring and an unsaturated aliphatic heterocycle comprising 1-3 heteroatoms (preferably 1-2 heteroatoms), which are independently selected from N, O, S.

In certain specific embodiments of the present application, the compound represented by formula (IV) provided in the present application has any one of the structures represented by formula (IV-a) to formula (IV-h):

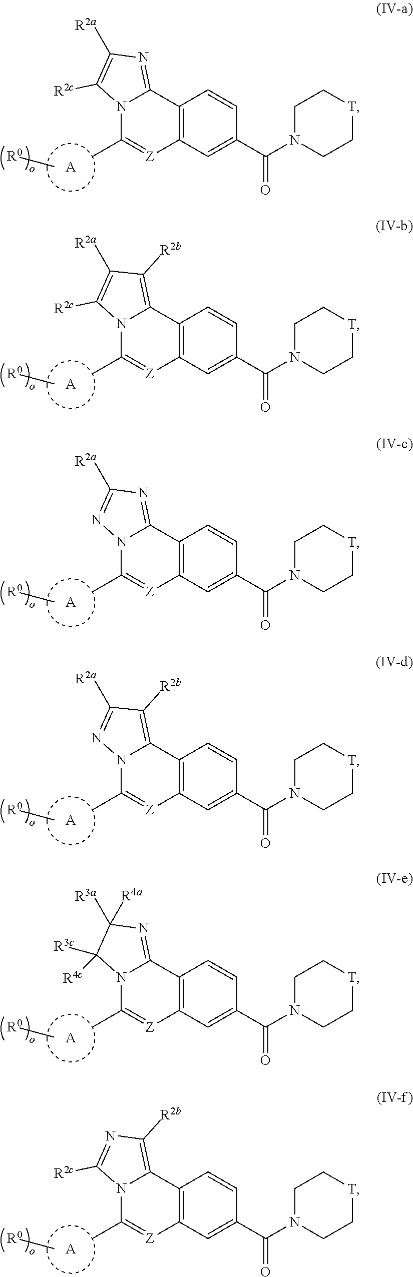

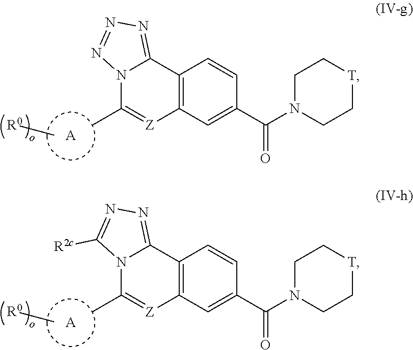

wherein ring A, o, R⁰, Z, R^{2a}, R^{2b}, R^{2c}, R^{3a}, R^{3c}, R^{4a}, and R^{4c} are the same as defined above, and T is selected from a covalent bond, O, CH₂, CHF, and CF₂, preferably selected from CH₂, CHF, and CF₂.

For the compounds represented by formula (I), formula (II), formula (III), formula (IV), formula (II-a), formula (III-a), formula (III-b), formula (III-c), formula (III-d), formula (III-e), formula (III-f), formula (III-g), formula (III-h), formula (III-i), formula (IV-a), formula (IV-b), formula (IV-c), formula (IV-d), formula (IV-e), formula (IV-f), formula (IV-g), and formula (IV-h) provided in the present application, in certain embodiments, the R⁰ is each independently selected from deuterium, tritium, nitro, hydroxyl, mercapto, fluoro, chloro, bromo, iodo, cyano, —O, ═NH, NHCH₃, —CH₂CH₃, ═NCH(CH₃)₂, —NH₂, —NHCH₃, —N(CH₃)₂, —NHCH₂CH₃, —N(CH₂CH₃)₂, —C(O)OCH₃, —C(O)OCH₂CH₃, —C(O)O(CH₂)₂CH₃, —C(O)OCH(CH₃)₂, —OC(O)CH₃, —OC(O)CH₂CH₃, —OC(O) (CH₂)₂CH₃, —OC(O)CH(CH₃)₂, an aldehyde group, carboxyl, —COCH₃, —COCH₂CH₃, —CO(CH₂)₂CH₃, —COCH(CH₃)₂, —CONH₂, —CONHCH₃, —CON(CH₃)₂, —NHCOH, —NHCOCH₃, —N(CH₃)COCH₃, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, trifluoromethoxy, trifluoroethoxy, trichloromethoxy, trichloroethoxy, piperazinyl, N-methylpiperazinyl, morpholinyl, piperidinyl, thiomorpholinyl, oxazolidinyl, isoxazolidinyl, thiazolidinyl, isothiazolidinyl, cyclopropoxy, cyclopropylmethoxy, cyclopropyl, cyclobutyl, cyclopentyl, and cyclohexyl; preferably, the R⁰ is each independently selected from fluoro, cyano, ═O, —NH₂, —N(CH₃)₂, —C(O)OCH₂CH₃, carboxyl, —COCH₃, methyl, piperazinyl, N-methylpiperazinyl, morpholinyl, and piperidinyl.

For the compounds represented by formula (I), formula (II), formula (III), formula (IV), formula (II-a), formula (III-a), formula (III-b), formula (III-c), formula (III-d), formula (III-e), formula (III-f), formula (III-g), formula (III-h), formula (III-i), formula (IV-a), formula (IV-b), formula (IV-c), formula (IV-d), formula (IV-e), formula (IV-f), formula (IV-g), and formula (IV-h) provided in the present application, in certain embodiments, the ring A is selected from benzene ring, naphthalene ring, a 5-10 membered aromatic heterocycle comprising 1-3 heteroatoms, a 6 membered unsaturated aliphatic heterocycle comprising 1-2 heteroatoms, or an 8-14 membered fused ring consisting of benzene ring and an unsaturated aliphatic heterocycle comprising 1-2 heteroatoms, which are independently selected from N, O, S; further, the ring A is selected from

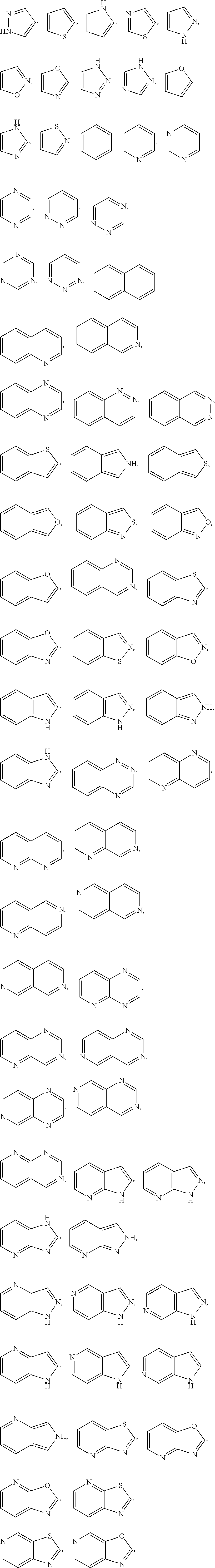

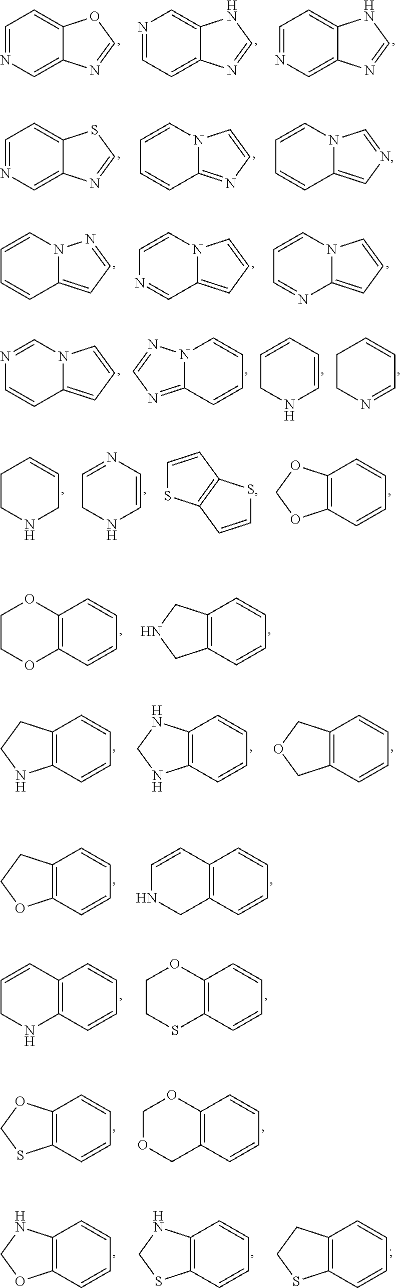

preferably selected from

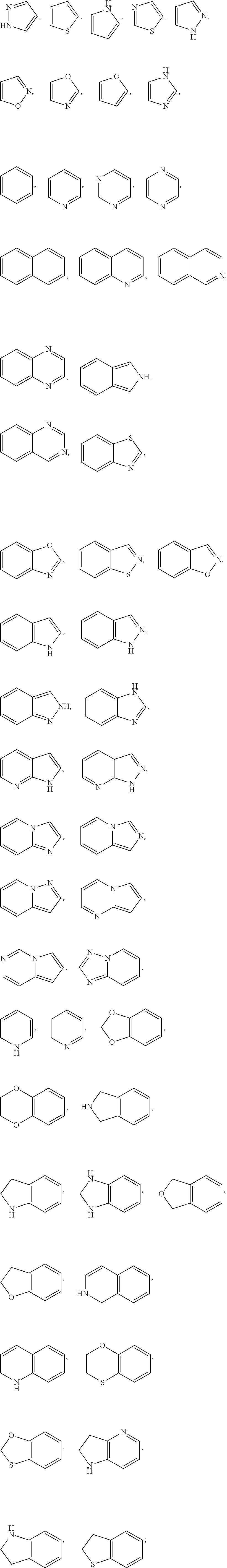

more preferably selected from

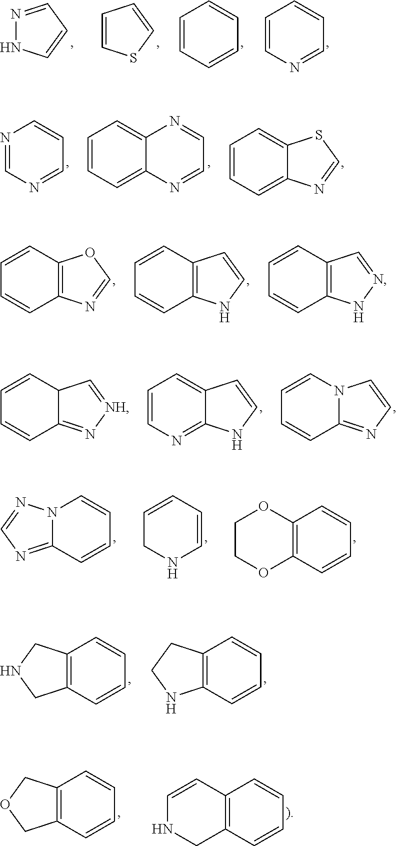

For the compounds represented by formula (I), formula (II), formula (III), formula (IV), formula (II-a), formula (III-a), formula (III-b), formula (III-c), formula (III-d), formula (III-e), formula (III-f), formula (III-g), formula (III-h), formula (III-i), formula (IV-a), formula (IV-b), formula (IV-c), formula (IV-d), formula (IV-e), formula (IV-f), formula (IV-g), and formula (IV-h), stereoisomers thereof, tautomers thereof or mixture forms thereof, or pharmaceutically acceptable salts thereof, or solvates (e.g., hydrates) thereof, or prodrugs thereof provided in the present application, in certain embodiments, the Z is selected from N, the o is selected from 0, 1, or 2, the R⁰ is each independently selected from deuterium, tritium, nitro, hydroxyl, mercapto, halogen, cyano, ═O, imino, an amine group, an ester group, an aldehyde group, carboxyl, carbonyl, amido, C₁-C₆ alkyl, C₁-C₆ haloalkyl, C₁-C₆ alkoxy, C₁-C₆ haloalkoxy (such as the R⁰ is each independently selected from fluoro, cyano, ═O, —NH₂, —N(CH₃)₂, —C(O)OCH₂CH₃, carboxyl, —COCH₃, methyl), the ring A is selected from benzene ring, naphthalene ring, a 5-10 membered aromatic heterocycle comprising 1-3 heteroatoms, a 6 membered unsaturated aliphatic heterocycle comprising 1 heteroatom, or an 8-14 membered fused ring consisting of benzene ring and an unsaturated aliphatic heterocycle comprising 1-2 heteroatoms, which are independently selected from N, O, S (such as the ring A is selected from

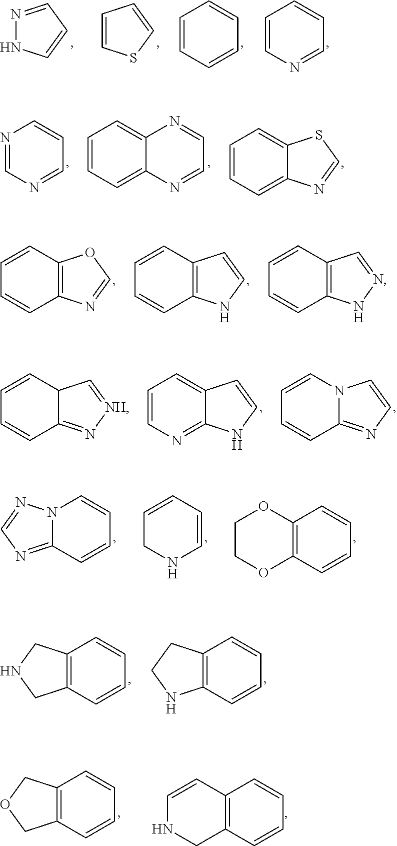

In some specific embodiments of the present application, in a compound represented by formula (I), a stereoisomer thereof, a tautomer thereof or a mixture form thereof, or a pharmaceutically acceptable salt thereof, or a solvate (e.g., a hydrate) thereof, or a prodrug thereof: ring A is

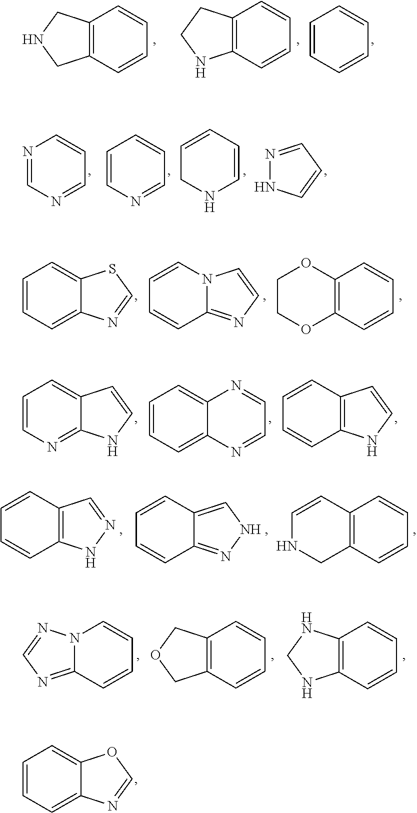

is selected from 0, 1, and 2,
R⁰ is each independently selected from deuterium, tritium, imino, an amine group, C₁-C₆ alkyl (preferably C₁-C₈alkyl, C₁-C₄ alkyl, or C₁-C₃ alkyl, such as methyl, ethyl, propyl), ═O, —C(O)OCH₃, —C(O)OCH₂CH₃, —C(O)O(CH₂)₂CH₃, —C(O)OCH(CH₃)₂, carboxyl, —NH₂, —NHCH₃, —N(CH₃)₂, —NHCH₂CH₃, —N(CH₂CH₃)₂, and cyano,

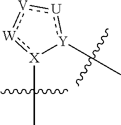

is selected from

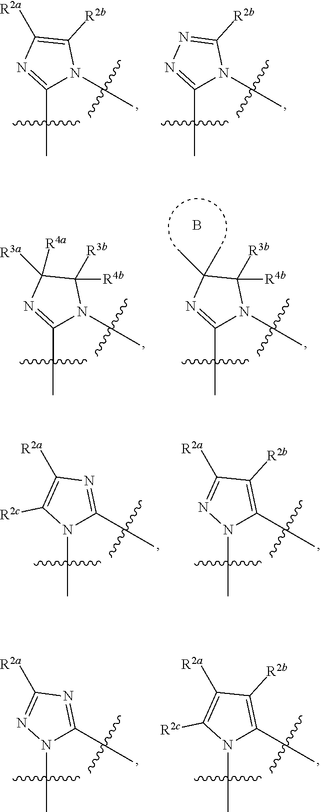

the ring B is selected from cyclopropyl and cyclobutyl, and the R^{2a}, R^{2b}, R^{2c}, R^{3a}, R^{3b}, R^{4a}, and R^{4b} are each independently selected from hydrogen, deuterium, tritium, methyl, ethyl, n-propyl, isopropyl, n-butyl, isobutyl, tert-butyl, trifluoromethyl, trifluoroethyl, trichloromethyl, trichloroethyl, and cyclopropyl,
◯ represents an aromatic ring, Z is selected from N,
R⁷ and R⁸ are each independently C₁-C₆ alkyl, preferably C₁-C₅ alkyl, C₁-C₄ alkyl, or C₁-C₃ alkyl (such as methyl, ethyl, propyl), or R⁷ and R⁸, together with the nitrogen atom to which they are attached, form

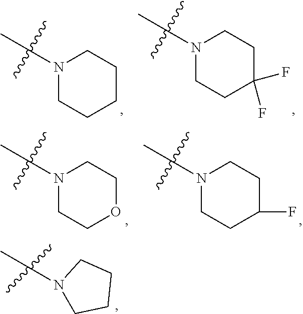

(preferably,

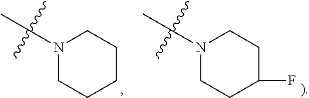 [SMILES: *N1CCCCC1 ; *N1CCC(F)CC1]

In some specific embodiments of the present application, the present application provides the following compounds, stereoisomers thereof, tautomers thereof or mixture forms thereof, or pharmaceutically acceptable salts thereof, or solvates (e.g., hydrates) thereof, or prodrugs thereof:

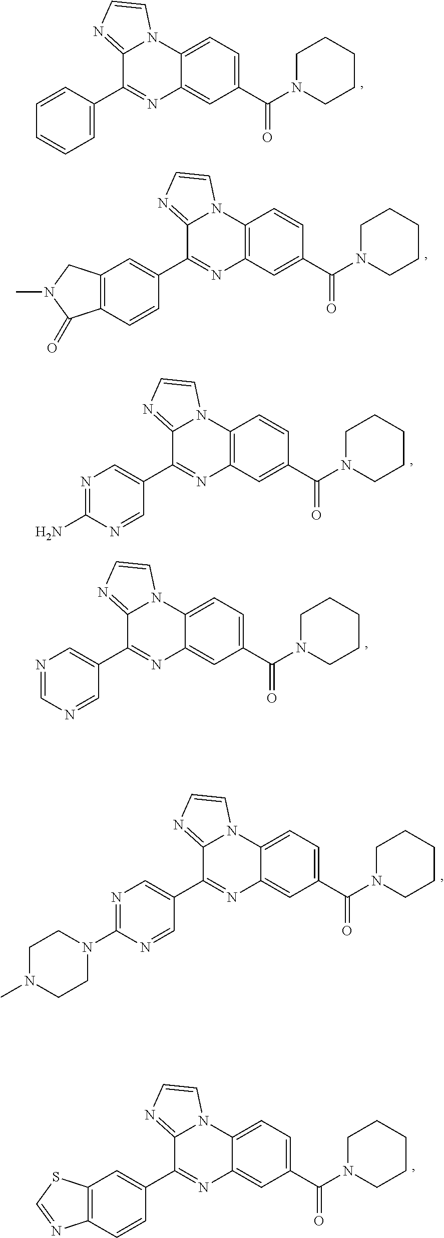

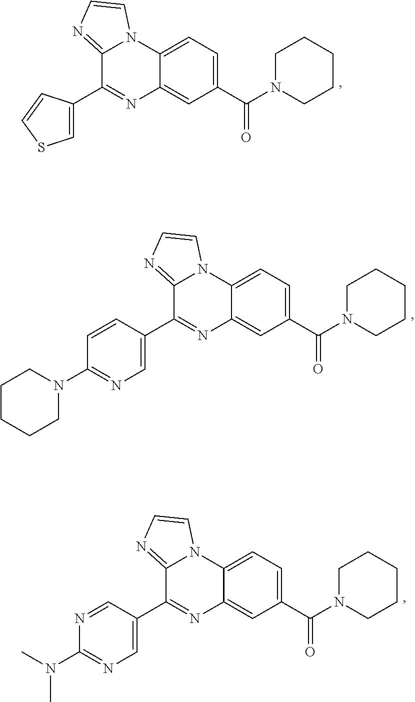 [SMILES: O=C(c1ccc2c(c1)nc(-c1ccsc1)c1nccn12)N1CCCCC1 ; O=C(c1ccc2c(c1)nc(-c1ccc(N3CCCCC3)nc1)c1nccn12)N1CCCCC1 ; CN(C)c1ncc(-c2nc3cc(C(=O)N4CCCCC4)ccc3n3ccnc23)cn1]

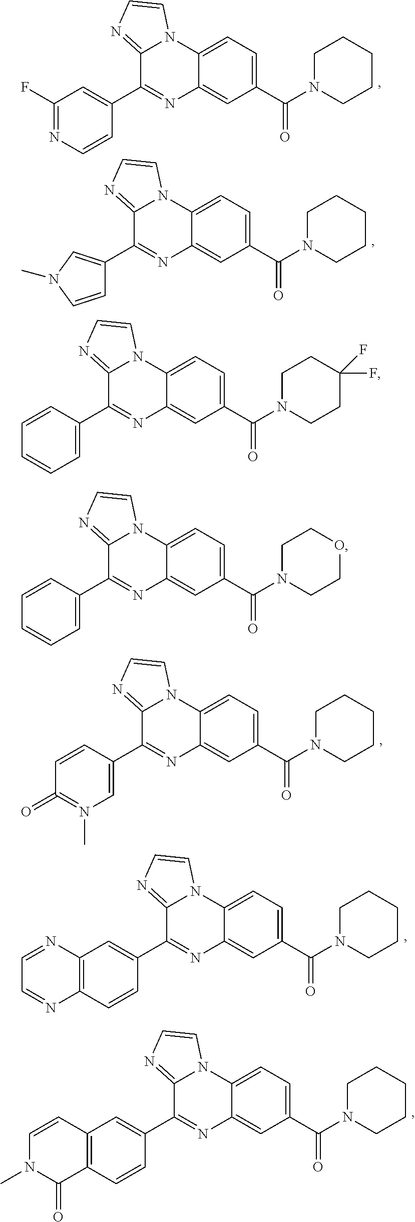

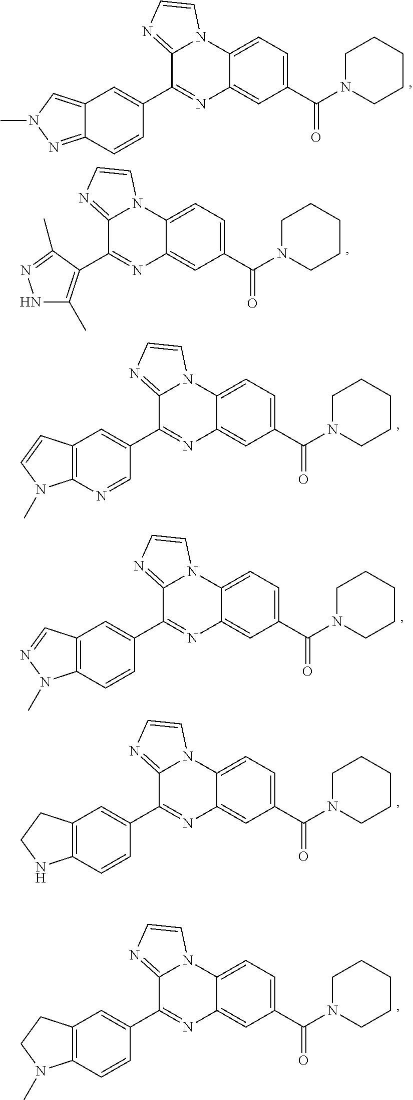

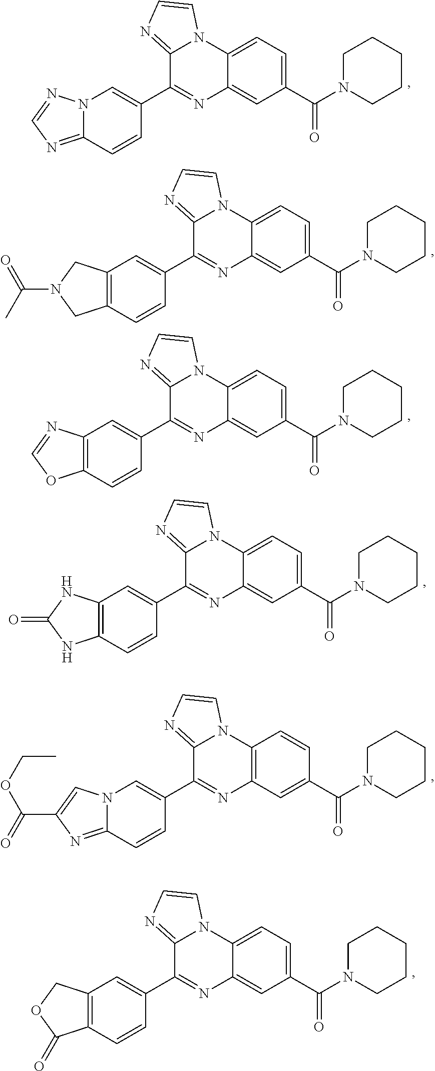

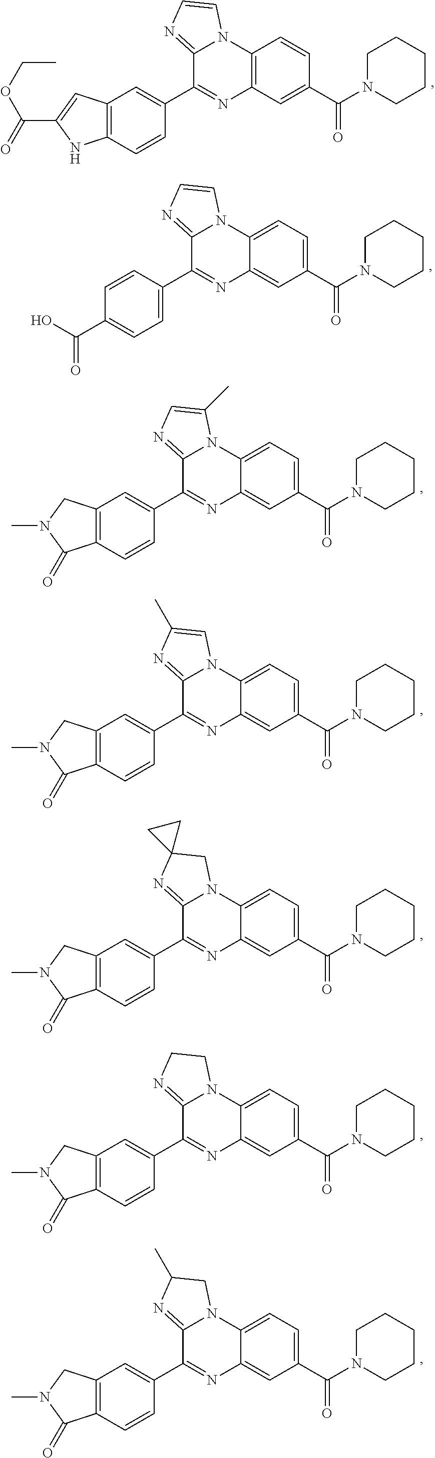

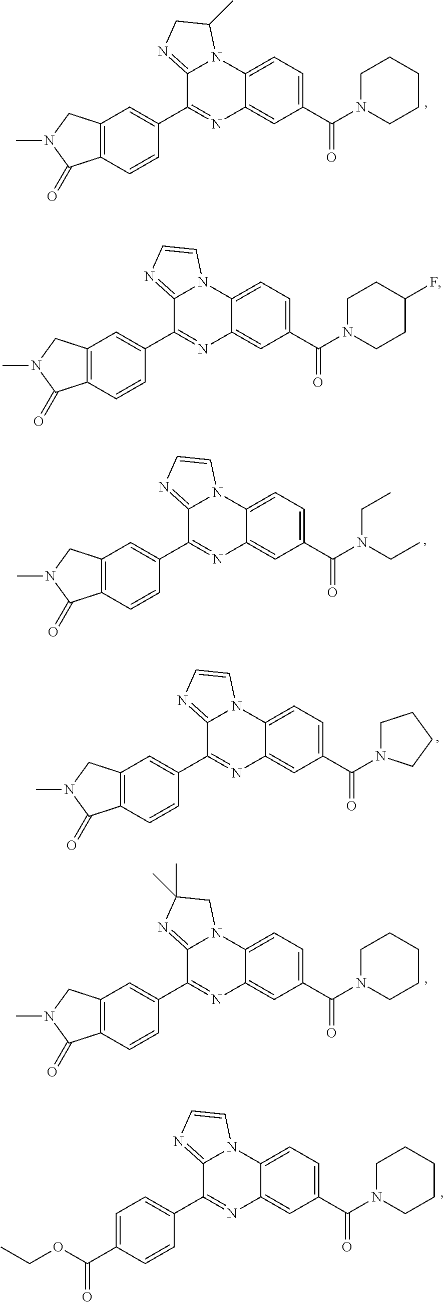

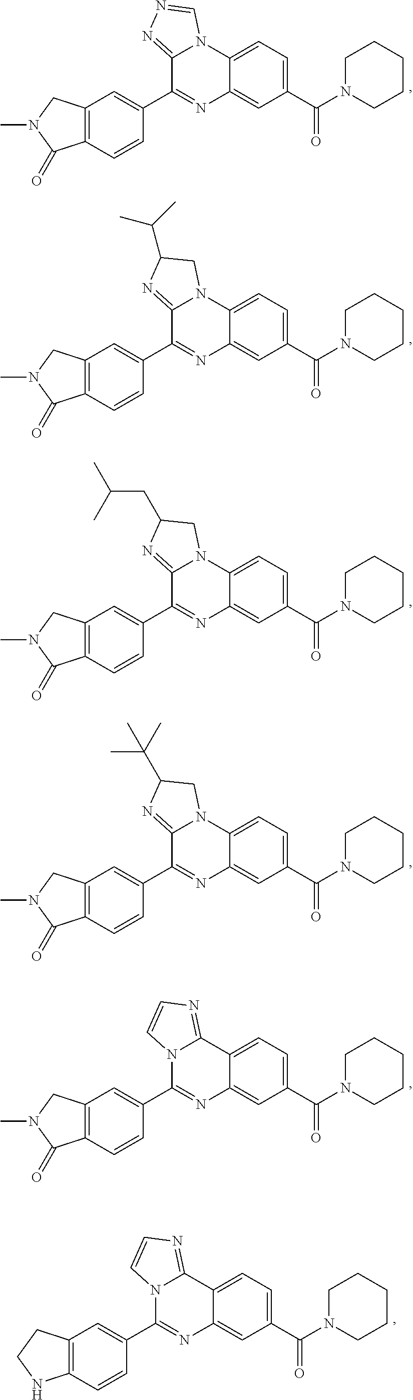 [SMILES: CN1Cc2cc(-c3nc4cc(C(=O)N5CCCCC5)ccc4n4cnnc34)ccc2C1=O ; CC(C)C1CN2C(=N1)C(c1ccc3c(c1)CN(C)C3=O)=Nc1cc(C(=O)N3CCCCC3)ccc12 ; CC(C)CC1CN2C(=N1)C(c1ccc3c(c1)CN(C)C3=O)=Nc1cc(C(=O)N3CCCCC3)ccc12 ; CN1Cc2cc(C3=Nc4cc(C(=O)N5CCCCC5)ccc4N4CC(C(C)(C)C)N=C34)ccc2C1=O ; CN1Cc2cc(-c3nc4cc(C(=O)N5CCCCC5)ccc4c4nccn34)ccc2C1=O ; O=C(c1ccc2c(c1)nc(-c1ccc3c(c1)CCN3)n1ccnc21)N1CCCCC1]

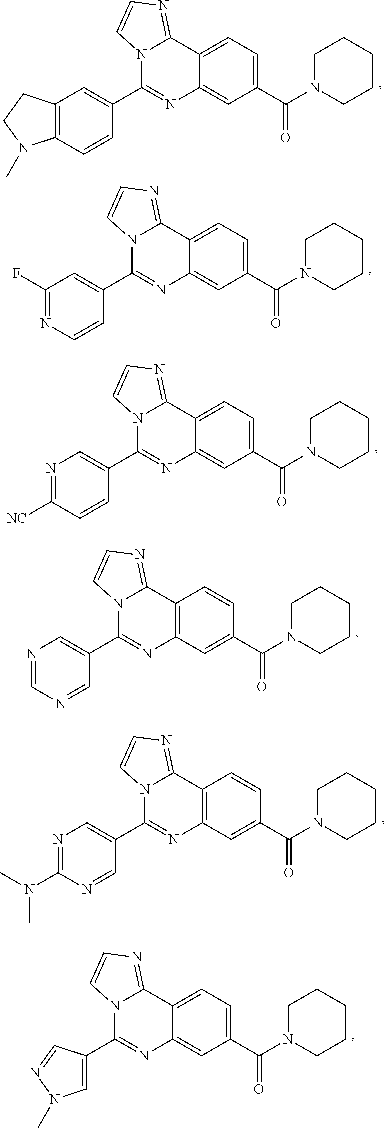

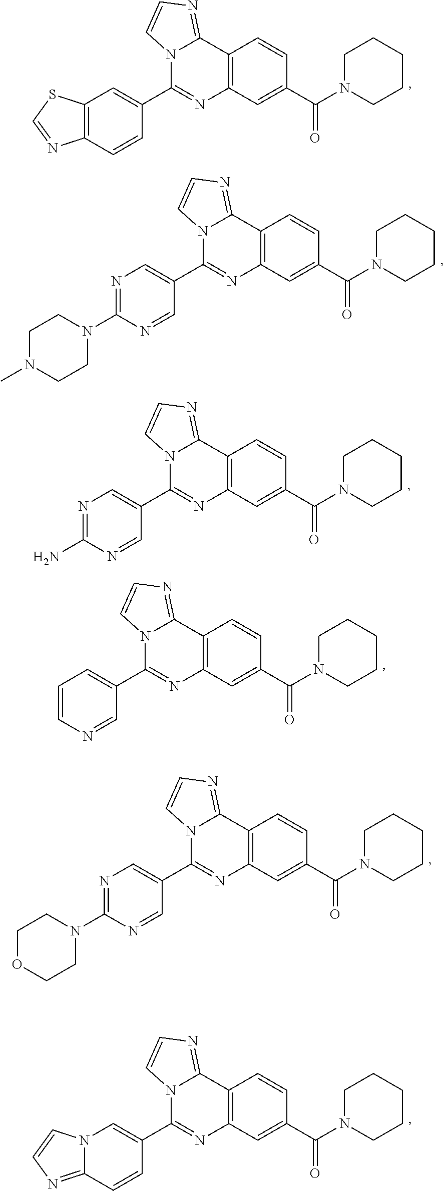

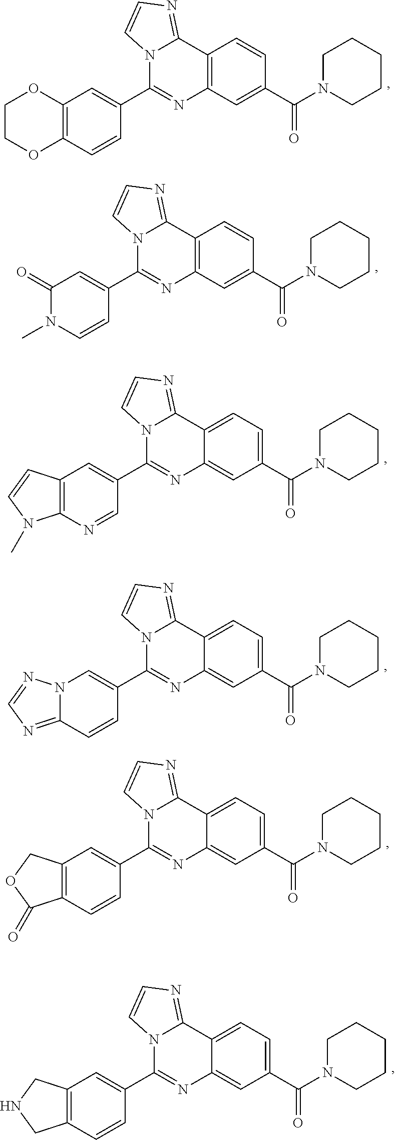

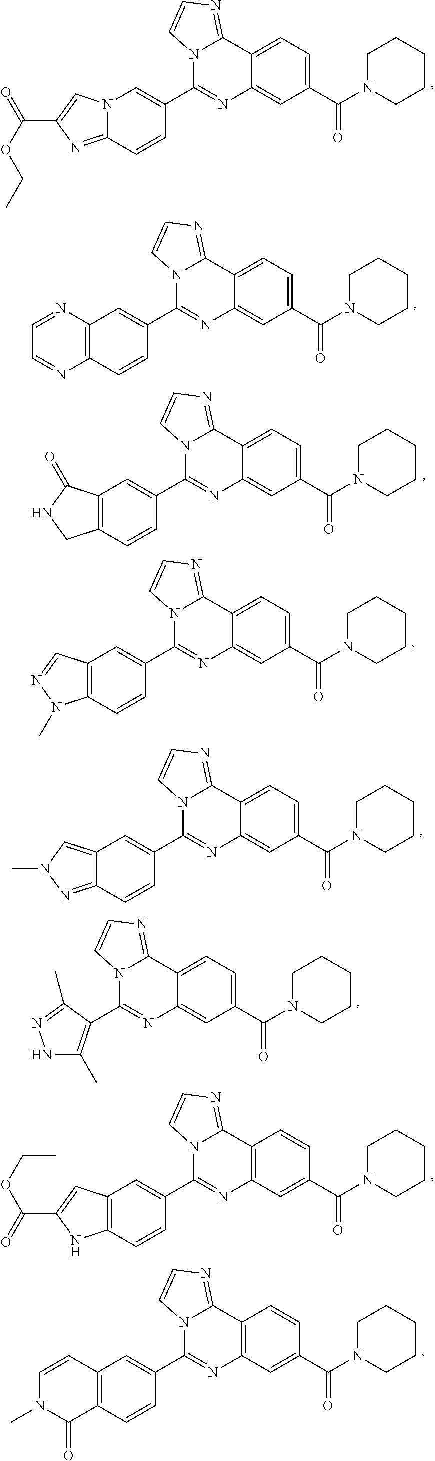

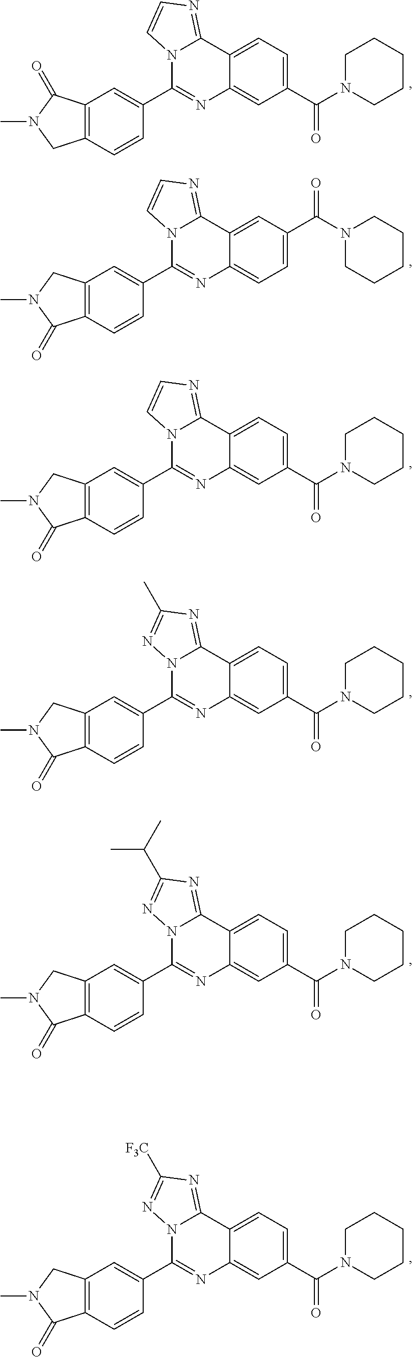

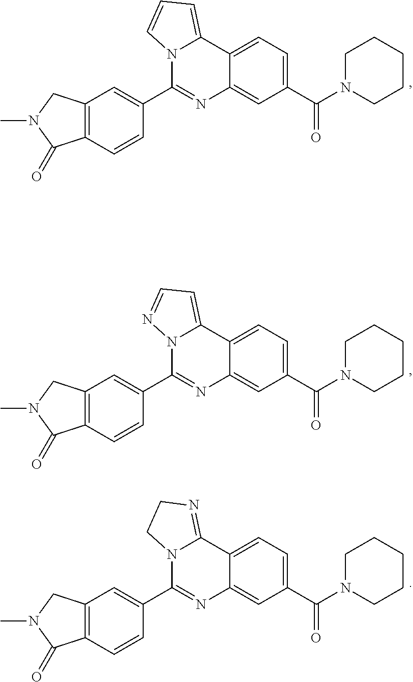 [SMILES: CN1Cc2cc(-c3nc4cc(C(=O)N5CCCCC5)ccc4c4cccn34)ccc2C1=O ; CN1Cc2cc(-c3nc4cc(C(=O)N5CCCCC5)ccc4c4ccnn34)ccc2C1=O ; CN1Cc2cc(C3=Nc4cc(C(=O)N5CCCCC5)ccc4C4=NCCN34)ccc2C1=O]

The present application further covers embodiments that are obtained by any combination, deletion or conversion of the above embodiments.

Another aspect of the present application provides a pharmaceutical composition, comprising at least one of the above-mentioned compounds, the stereoisomers, tautomers or mixture forms thereof, or the pharmaceutically acceptable salts thereof, or the solvates thereof, or the prodrugs thereof, and at least one pharmaceutically acceptable excipient.

Another aspect of the present application provides use of the above-mentioned compounds, or the stereoisomers, tautomers or mixture forms thereof, or the pharmaceutically acceptable salts thereof, or the solvates thereof, or the prodrugs thereof, or the pharmaceutical composition in the preparation of a medicament. Wherein the medicament is a 15-PGDH inhibitor which can be used for treating a disease associated with an undesirably increased activity level of 15-PGDH. Alternatively, the present application provides the above-mentioned compounds, or the stereoisomers, tautomers or mixture forms thereof, or the pharmaceutically acceptable salts thereof, or the solvates thereof, or the prodrugs thereof, or the pharmaceutical composition for use as a medicament. Alternatively, the present application provides a method of treating or preventing a disease associated with 15-PGDH, comprising administering to a subject in need thereof the above-mentioned compounds, or the stereoisomers, tautomers or mixture forms thereof, or the pharmaceutically acceptable salts thereof, or the solvates thereof, or the prodrugs thereof, or the pharmaceutical composition. The disease associated with 15-PGDH herein refers to a disease or complication thereof for which a clinically beneficial effect, such as remission, amelioration, cessation of progression, alleviation, or no further deterioration, is achieved by inhibiting the activity of 15-PGDH.

In certain specific embodiments, the medicament or the method is used for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, underactive bladder; or for promoting hair growth, pigmentation, tissue repair, tissue regeneration, implant in stem cell transplantation or bone marrow transplantation or organ transplantation, neurogenesis and neuronal cell death, muscle regeneration, and cervical ripening; or for enhancing resistance to the toxicity of chemotherapy and the toxicity of immunosuppressant.

The compounds, or the tautomers thereof or mixture forms thereof, or the pharmaceutically acceptable salts thereof, or the solvates thereof, or the prodrugs thereof, or the pharmaceutical compositions thereof provided in the present application further meet the requirement of small molecules for inhibiting the activity of 15-PGDH, have 15-PGDH enzyme inhibitory activity, and may have intracellular PGE2 up-regulatory activity. The research related to the present application is funded by the Sichuan Province Science and Technology Program.

### Definition

Unless otherwise stated, the following terms used in the specification and claims have the following meanings. A particular term or phrase shall not be considered uncertain or unclear in the absence of a specific definition, but should be understood according to its ordinary meaning.

“Chain hydrocarbyl” refers to aliphatic groups consisting of only carbon and hydrogen atoms linked in a chain form. The hydrocarbyl may be a saturated hydrocarbyl or an unsaturated hydrocarbyl; the chain may be linear or branched. C₁-C₁₀ chain hydrocarbyl used in the present application refers to a linear hydrocarbyl or a branched hydrocarbyl consisting of 1-10 (e.g., 1, 2, 3, 4, 5, 6, 7, 8, 9, 10, or a range value composed of any two of the preceding numerical values) carbon atoms, including saturated hydrocarbyl and unsaturated hydrocarbyl.

“Alkyl” refers to a saturated aliphatic chain hydrocarbyl group, comprising a linear or branched alkyl. For example, C₁-C₆ alkyl used in the present application refers to linear alkyl or branched alkyl consisting of 1-6 (e.g., 1, 2, 3, 4, 5, 6, or a range value composed of any two of the preceding numerical values) carbon atoms. Typical alkyl includes, but is not limited to, methyl, ethyl, n-propyl, isopropyl, n-butyl, isobutyl, tert-butyl, n-pentyl, isopentyl, tert-pentyl, n-hexyl, etc.

“Alkoxy” refers to —O-alkyl; C₁-C₆ alkoxy used herein refers to a linear or branched alkoxy group consisting of 1 to 6 (e.g., 1, 2, 3, 4, 5, 6, or a range value composed of any two of the preceding numerical values) carbon atoms. Typical alkoxy includes, but is not limited to, methoxy, ethoxy, n-propoxy, isopropoxy, n-butoxy, isobutoxy, tert-butoxy, n-pentyloxy, isopentyloxy, tert-pentyloxy, n-hexyloxy, etc.

“Ring” refers to any cyclic covalently closed structure, including, for example, a carbocycle (e.g., an aromatic or alicyclic ring), or a heterocycle (e.g., an aromatic or aliphatic heterocycle). The carbocycle refers to a ring only consisted of carbon atoms, and the heterocycle refers to a closed structure formed by covalently bonding carbon atoms and heteroatoms. Depending on the number of the ring, the “ring” may be monocyclic, bicyclic, tricyclic or polycyclic. When the ring is a bicyclic, tricyclic or polycyclic ring, the relationship between individual rings may include a fused ring, a spirocycle, or a bridged cycle.

“Heteroatom” refers to any atom, other than a carbon atom, that can be covalently bonded to a carbon atom. Common heteroatoms include, but are not limited to, O, S, N, P, Si, etc.

The “membered” refers to the number of skeleton atoms constituting a ring. A typical 5 membered ring may include, but is not limited to, cyclopentane, pyrrole, imidazole, thiazole, furan, thiophene, and the like, and a typical 6 membered ring includes, but is not limited to, cyclohexane, pyridine, pyran, pyrazine, thiapyran, pyridazine, pyrimidine, benzene, etc.

“Alicyclic ring” or “alicyclic group” refers to a saturated or partially unsaturated aliphatic carbocyclic group. The saturated aliphatic carbocycle is referred to as, for example, a saturated alicyclic ring, and may also be referred to as a “cycloalkyl”; the partially unsaturated carbocycle may be referred to as, for example, an unsaturated alicyclic ring. The alicyclic ring may be a monocyclic ring, a spirocycle, a fused ring or a bridged cycle, for example, a 3-8 membered alicyclic ring refers to an aliphatic carbocyclic group consisting of 3-8 skeleton carbon atoms. A typical alicyclic structure includes, but is not limited to:

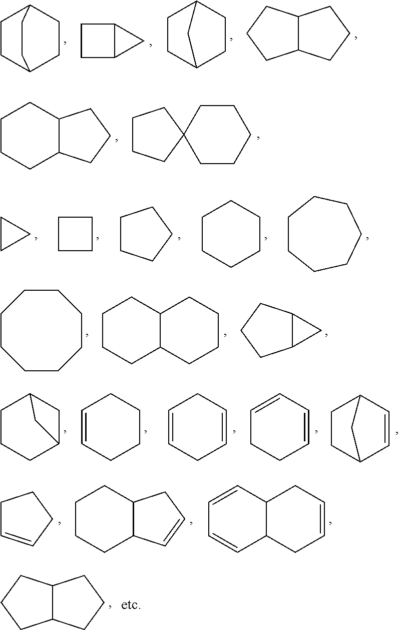

“Aliphatic heterocycle” or “aliphatic heterocyclic group” refers to a nonaromatic cyclic group formed by replacing carbon atom(s) in an alicyclic ring with one or more heteroatoms. The aliphatic heterocycle or aliphatic heterocyclic group may include a saturated aliphatic heterocycle and an unsaturated aliphatic heterocycle. For example, a 3-12 membered aliphatic heterocyclic group refers to a nonaromatic cyclic group comprising one or more heteroatoms consisting of 3-12 (e.g., 3, 4, 5, 6, 7, 8, 9, 10, 11, 12, or a range value composed of any two of the preceding numerical values) skeleton atoms, and may be a saturated aliphatic heterocyclic group and an unsaturated aliphatic heterocyclic group.

“Saturated alicyclic ring”, is also referred to as “cycloalkyl”, is an aliphatic cyclic group consisting of saturated carbon atoms as the skeleton. 3-8 membered cycloalkyl used in the application refers to a cyclic alkyl consisting of 3-8 (e.g., 3, 4, 5, 6, 7, 8, or a range value composed of any two of the preceding numerical values) carbon atoms. A typical cycloalkyl includes, but is not limited to, cyclopropyl, cyclobutyl, cyclopentyl, cyclohexyl, bicyclo[2,1,1]hexyl, cycloheptyl, and the like.

“Saturated aliphatic heterocycle”, also known as “heterocycloalkyl”, means that the carbon atoms in the aliphatic heterocycle that form the ring skeleton are all saturated. For example, a 3-12 membered heterocycloalkyl used in the present application refers to a nonaromatic cyclic group consisting of 3-12 (e.g., 3, 4, 5, 6, 7, 8, 9, 10, 11, 12, or a range value composed of any two of the preceding numerical values) atoms constituting the ring skeleton, wherein the atoms constituting the ring skeleton consist of saturated carbon atoms and heteroatoms. A typical saturated aliphatic heterocycle includes, but is not limited to:

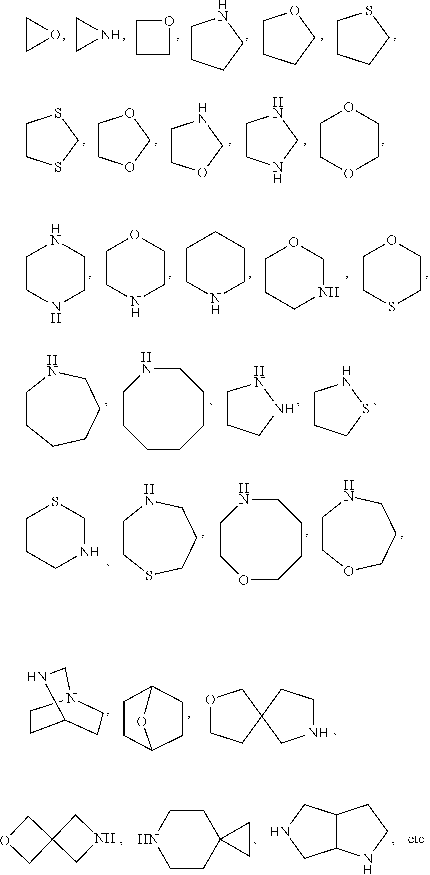

“Unsaturated aliphatic heterocycle” or “unsaturated aliphatic heterocyclyl” refers to a non-aromatic cyclic structure containing some unsaturated atoms as a ring skeleton in an aliphatic heterocycle. For example, in certain embodiments of the present application, the “unsaturated aliphatic heterocycle” means that the skeleton of the aliphatic heterocycle contains unsaturated carbon atoms. A 3-12 membered unsaturated aliphatic heterocycle used in the present application refers to a nonaromatic cyclic group consisting of 3-12 (e.g., 3, 4, 5, 6, 7, 8, 9, 10, 11, 12, or a range value composed of any two of the preceding numerical values) skeleton atoms, wherein the atoms constituting the ring skeleton include saturated carbon atoms, unsaturated carbon atoms, and heteroatoms, and a typical unsaturated aliphatic heterocycle includes, but is not limited to:

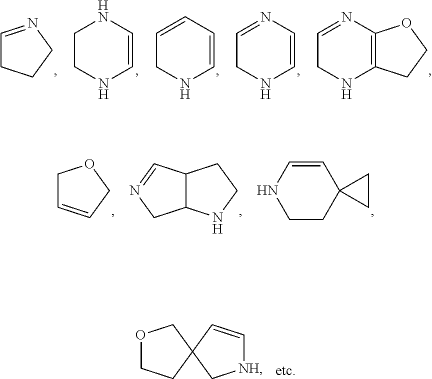

“Aromatic ring” or “aryl” refers to a completely unsaturated carbocycle with a planar ring having a delocalized π-electron system and containing 4n+2π electrons, where n is an integer. The aromatic ring may consist of six, eight, ten, or more than ten carbon atoms, and may be a monocyclic ring or a polycyclic ring (e.g., a bicyclic ring, a tricyclic ring). Common aromatic ring includes, but is not limited to, benzene ring, naphthalene ring, phenanthrene ring, anthracene ring, tetrabenzene, pyrene ring, pentabenzene, and the like. As used in the present application, a 6-10 membered aromatic ring or a 6-10 membered aryl refers to an aromatic ring group consisting of 6-10 (e.g., 6, 7, 8, 9, 10, or a range value composed of any two of the preceding numerical values) skeleton carbon atoms.

“Aromatic heterocycle” or “heteroaryl” refers to an aromatic cyclic structure formed by replacing carbon atoms in the aromatic ring with one or more heteroatoms, and a typical aromatic heterocycle or heteroaryl includes, but is not limited to:

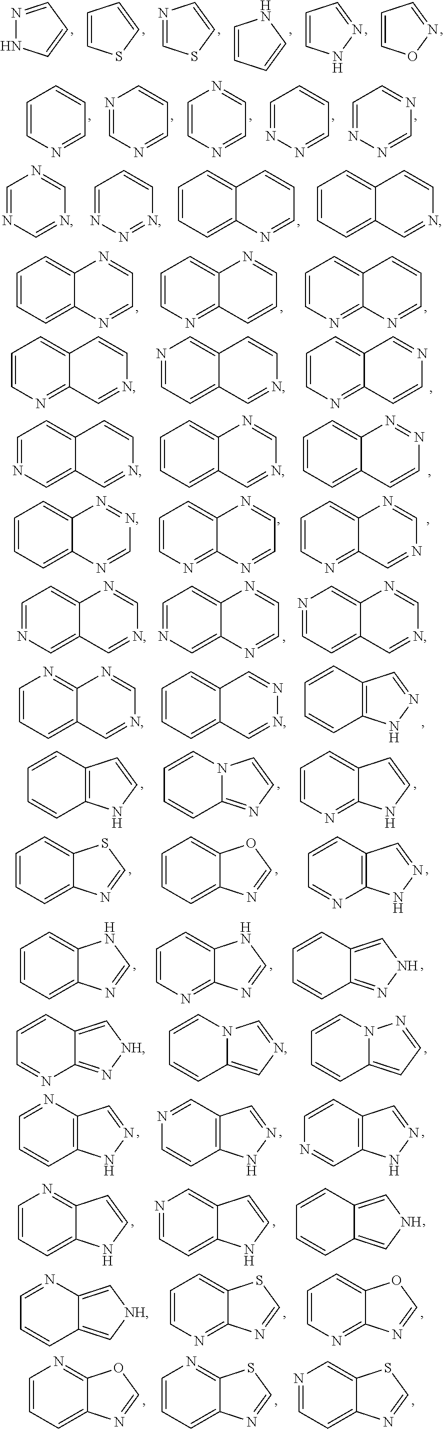

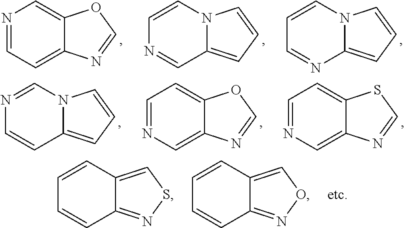

A 5-10 membered aromatic heterocycle or 5-10 membered heteroaryl used in the present application refers to an aromatic ring group comprising heteroatoms consisting of 5-10 (e.g., 5, 6, 7, 8, 9, 10, or a range value composed of any two of the preceding numerical values) skeleton atoms.

“Fused ring” refers to a cyclic structure formed by sharing two adjacent ring atoms between rings. The fused ring may be a polycyclic ring (such as a bicyclic ring, a tricyclic ring).

“Fused ring consisted of an aromatic ring and an unsaturated aliphatic heterocycle” in the present application refers to a fused ring structure formed by sharing two adjacent ring atoms between the aromatic ring and the unsaturated aliphatic heterocycle; “fused ring consisted of an aromatic heterocycle and an unsaturated aliphatic heterocycle” refers to a fused ring structure formed by sharing two adjacent ring atoms between the aromatic heterocycle and the unsaturated aliphatic heterocycle. “7-12 membered fused ring consisted of an aromatic heterocycle and an unsaturated aliphatic heterocycle” in the present application refers to a fused ring structure having 7-12 (e.g., 7, 8, 9, 10, 11, 12, or a range value composed of any two of the preceding numerical values) skeleton ring atoms formed by sharing two adjacent ring atoms between the unsaturated aliphatic heterocycle and the aromatic heterocycle. “7-12 membered fused ring consisted of an aromatic ring and an unsaturated aliphatic heterocycle” in the present application refers to a fused ring structure having 7-12 (e.g., 7, 8, 9, 10, 11, 12, or a range value composed of any two of the preceding numerical values) skeleton ring atoms formed by sharing two adjacent ring atoms between the unsaturated aliphatic heterocycle and the aromatic ring. A common fused ring consisted of an aromatic ring and an unsaturated aliphatic heterocycle includes, but is not limited to:

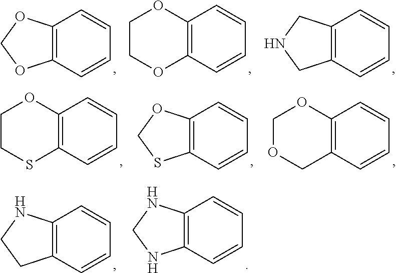

A common fused ring consisted of an aromatic heterocycle and an unsaturated aliphatic heterocycle includes, but is not limited to:

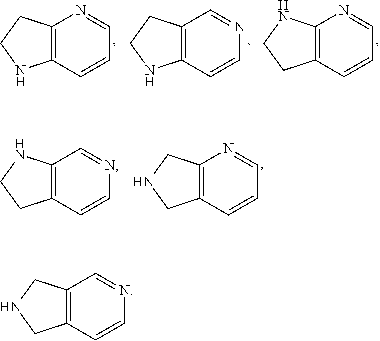

The “halogen” or “halo” refers to fluorine, chlorine, bromine or iodine.

“Haloalkyl” means that at least one hydrogen in an alkyl group is replaced by a halogen atom, and C₁-C₆ haloalkyl, as used in this application, means linear or branched alkyl consisting of 1-6 (e.g., 1, 2, 3, 4, 5, 6, or a range value composed of any two of the preceding numerical values) carbon atoms and at least one hydrogen in the alkyl is arbitrarily replaced by a halogen atom.

“Haloalkoxy” means that at least one hydrogen in an alkoxy group is replaced by a halogen atom, and a C₁-C₆ haloalkoxy, as used in this application, means a linear or branched alkoxy consisting of 1-6 (e.g., 1, 2, 3, 4, 5, 6, or a range value composed of any two of the preceding numerical values) carbon atoms and at least one hydrogen in the alkoxy is arbitrarily replaced by a halogen atom.

“Amine group” or “amine” means having a chemical structure of —NR^{S}R^{T}, wherein R^{S} and R^{T} are each independently selected from hydrogen, deuterium, tritium, alkyl, and cycloalkyl. Common “amine group” includes, but is not limited to, —NH₂, —NHCH₃, —N(CH₃)₂, —NHCH₂CH₃, —N(CH₂CH₃)₂.

“Imino” or “imine” means having a chemical structure of ═NR^{W}, wherein R^{W} is selected from hydrogen, deuterium, tritium, C₁-C₆ alkyl, or 3-12 membered cycloalkyl. Common imino includes, but is not limited to, ═NH, ═NCH₃, ═CH₂CH₃, ═NCH(CH₃)₂.

“Carbonyl” means having a chemical structure of —COR^{Z}, wherein R^{Z} is selected from alkyl, cycloalkyl, and heterocycloalkyl. Common carbonyl includes, but is not limited to, —COCH₃, —COCH₂CH₃, —CO(CH₂)₂CH₃, —COCH(CH₃)₂.

“Amide” or “amido” means having a chemical structure of —C(O)NR^{U}R^{V} or —NR^{U}C(O)R^{V}, wherein R^{U} and R^{V} are each independently selected from hydrogen, deuterium, tritium, alkyl, cycloalkyl, and heterocycloalkyl. Common amido includes, but is not limited to, —CONH₂, —CONHCH₃, —CON(CH₃)₂, —NHCOH, —NHCOCH₃, —N(CH₃)COCH₃.

“Ester group” means having a chemical structure of a formula of —C(O)OR^{a} or —OC(O)R^{b}, wherein R^{a} and R^{b} are selected from alkyl, cycloalkyl, and heterocycloalkyl. Common ester group includes, but is not limited to, —C(O)OCH₃, —C(O)OCH₂CH₃, —C(O)O(CH₂)₂CH₃, —C(O)OCH(CH₃)₂, —OC(O)CH₃, —OC(O)CH₂CH₃, —OC(O) (CH₂)₂CH₃, —OC(O)CH(CH₃)₂.

“Substituted” means that one or more hydrogen atoms in a group are substituted independently by a corresponding number of substituents. It goes without saying that, the substituents are only in their possible chemical positions, and those of skills in the art are able to determine (either experimentally or theoretically) possible or impossible substitutions without undue effort. For example, an amino or hydroxyl with a free hydrogen may be unstable when binds to a carbon atom with an unsaturated (e.g. olefinic) bond. Each is independently selected from alkyl, cycloalkyl, aryl, heteroaryl, heterocycloalkyl, hydroxy, alkoxy, alkylthio, aryloxy, nitro, acyl, halogen, haloalkyl, amino, and the like.

The optional groups or substituents in the general formula compounds of the present application being “each independently selected from” means that when the groups or substituents appear at multiple positions at the same time, the groups or substituents at each position may be the same or different. For example, when there are multiple Ros in the same general formula compound, each R⁰ may be the same or different and may be selected from the same or different specific groups.

“Inhibitor” refers to a substance reducing the activity of an enzyme.

“Optional” or “optionally” means that the event or circumstance subsequently described may, but not necessarily, occur, and the description includes a situation when the event or circumstance does or does not occur. For example, “optionally substituted” includes substituted or unsubstituted, e.g., “a heterocyclic group optionally substituted by an alkyl” means that the alkyl may, but not necessarily, be present, and the description includes a situation in which the heterocyclic group is substituted by the alkyl and a situation in which the heterocyclic group is not substituted by the alkyl.

“Pharmaceutical composition” indicates a mixture comprising one or more of the compounds described herein, or a physiologically/pharmaceutically acceptable salt or prodrug thereof, with other chemical as other components, well components such as physiologically/pharmaceutically acceptable carriers and vehicles. The pharmaceutical composition is intended to facilitate administration to an organism and facilitate absorption of an active ingredient to exert the biological activity.

“Pharmaceutically acceptable” refers to those compounds, materials, compositions and/or dosage forms that are suitable for use in contact with human and animal tissues within a range of sound medical judgment, without undue toxicity, irritation, allergic response or other problems or complications, commensurate with a reasonable benefit/risk ratio. As pharmaceutically acceptable salts, mention may be made of, e.g., metal salts, ammonium salts, salts formed with organic bases, salts formed with inorganic acids, salts formed with organic acids, salts formed with basic or acidic amino acids, and the like.

“Tautomer” or “tautomeric form” refers to structural isomers of different energies that can be interconverted through low-energy barriers. For example, proton tautomer (also known as proton transfer tautomer) includes tautomerism via proton migration, such as keto-enol and imine-enamine isomerization. A specific example of a proton tautomer is imidazole moiety, wherein the proton can migrate between the two ring nitrogens. Valence tautomers include interconversion by recombination of some bonding electrons. Non-limiting examples of tautomers include, but are not limited to,

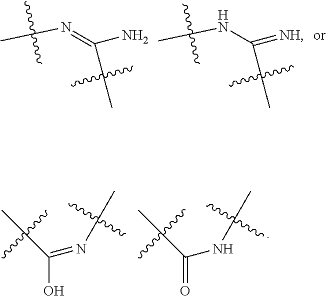 [SMILES: */N=C(\*)N ; *NC(*)=N ; */N=C(\*)O ; *NC(*)=O]

“Stereoisomer” refers to an isomer in which atoms or atomic groups within molecules are connected in the same order but have different spatial arrangements. It can be divided into several major categories: conformational isomers, cis-trans isomers, and chiral isomers.

Chiral isomers can be divided into two major categories: enantiomers and diastereoisomers. The spatial arrangement of atoms in the compound structure of “stereoisomer” is usually represented by wedge-shaped covalent bonds (a solid wedge-shaped bond “ [symbol] ”, a dashed wedge-shaped bond “ [symbol] ”), wherein the solid wedge-shaped bond indicates facing out of the paper, and the dashed wedge-shaped bond indicates facing into the paper.

“Enantiomer” refers to isomerism caused by different spatial configurations of the atoms of compounds having the same molecular formula and functional groups, and the compounds form stereoisomers that are mirror images of each other and cannot overlap. “Diastereoisomer” refers to isomerism caused by different spatial configurations of the atoms of compounds having the same molecular formula and functional groups, and the compounds are stereoisomers that do not exhibit a physical or mirror image relationship with each other.

In the present application, the linear covalent bond “—” in the compound structure may indicate that it is coplanar with the paper. In the present application, when there are stereoisomers of atoms connected by a linear covalent bond, the linear covalent bond indicates that the arrangement of the connected atoms may include being coplanar with the paper, facing out of the paper, facing into the paper, or a mixture of various arrangements.

Unless otherwise indicated, the terms “comprise, comprises and comprising” or their equivalents (contain, contains, containing, include, includes, including) used herein are open-ended mode expressions, and mean that other unspecified elements, components and steps may also be covered, in addition to the elements, components and steps listed. Unless otherwise indicated, all numbers used herein to denote amounts of ingredients, measurements, or reaction conditions should be understood to be modified in all cases by the term “about”. When associated with a percentage, the term “about” may indicate, for example, ±1%, preferably ±0.5%, more preferably ±0.1%. Unless otherwise specified clearly in the context, singular terms herein cover plural referents, and vice versa. Similarly, unless otherwise specified clearly in the context, the word “or” herein is intended to include “and”.

Apparently, according to the above contents of the application, in accordance with the ordinary technical knowledge and means in the field, under the premise of not departing from the above basic technical concepts of the application, a variety of other forms of modifications, substitutions or changes can also be made.

The abbreviations in the application have the meanings indicated below:

| | |
|---|---|
| NH₄OH or NH₃•H₂O represents ammonia water; | THF represents tetrahydrofuran; |
| DIPEA or DIEA represents N,N- | LiOH represents lithium hydroxide; |
| diisopropylethylamine; | |
| TEA represents triethylamine; | EtOH represents ethanol; |
| DCM represents dichloromethane; | MeCN or ACN represents acetonitrile; |
| POCl₃ represents phosphorus oxychloride; | CHCl₃ represents chloroform or |
| | trichloromethane; |
| DMF represents N,N-dimethylformamide; | [O] represents oxidation reaction; |
| TFA represents trifluoroacetic acid; | dioxane represents 1,4-dioxane; |
| NH₂NH₂ or N₂H₄•H₂O represents hydrazine | SOCl₂ represents thionyl chloride; |
| hydrate; | |
| Pd(dppf)₂Cl₂ represents [1,1′- | HATU represents N,N,N′,N′-tetramethyl-O- |
| bis(diphenylphosphino)ferrocene]dichloropalladium; | (7-azabenzotriazol-1-yl)uronium |
| | hexafluorophosphate; |
| dioxane/H₂O represents dioxane aqueous | EtO— represents ethoxy; |
| solution; | |
| Boc— represents tert-butyloxycarbonyl; | Triphosgene or BTC represents triphosgene; |
| NMP stands for N-methylpyrrolidone; | K₂CO₃ represents potassium carbonate; |
| DDQ represents 2,3-dichloro-5,6- | EtOH/H₂O represents ethanol aqueous |
| dicyanobenzoquinone; | solution; |
| Cs₂CO₃ represents cesium carbonate; | CO represents carbon monoxide. |

### EMBODIMENTS OF THE INVENTION

### Synthetic Methods

The present application also provides methods for synthesizing the compounds as described above. The synthetic methods of the present application are mainly based on the preparation methods reported in chemical literature or on the relevant synthesis using commercially available chemical reagents as starting materials.

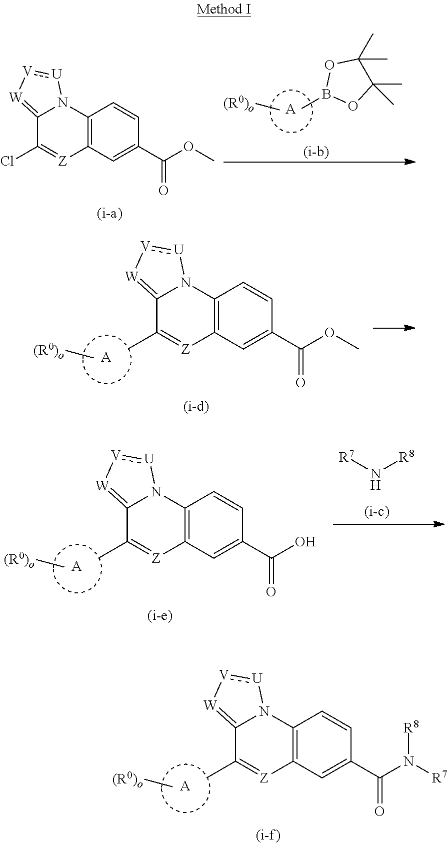

The steps are as follows:
a) the compound represented by formula (i-a) or a pharmaceutically acceptable salt thereof, such as the hydrochloride salt, is subjected to a reaction with the compound represented by formula (i-b) to obtain the compound represented by formula (i-d);
b) the compound represented by formula (i-d) is hydrolyzed to obtain the compound represented by formula (i-e);
c) the compound represented by formula (i-e) is subjected to a reaction with the compound represented by formula (i-c) to obtain the compound represented by formula (i-f).

Alternatively, Method I can also be carried out through the following steps,

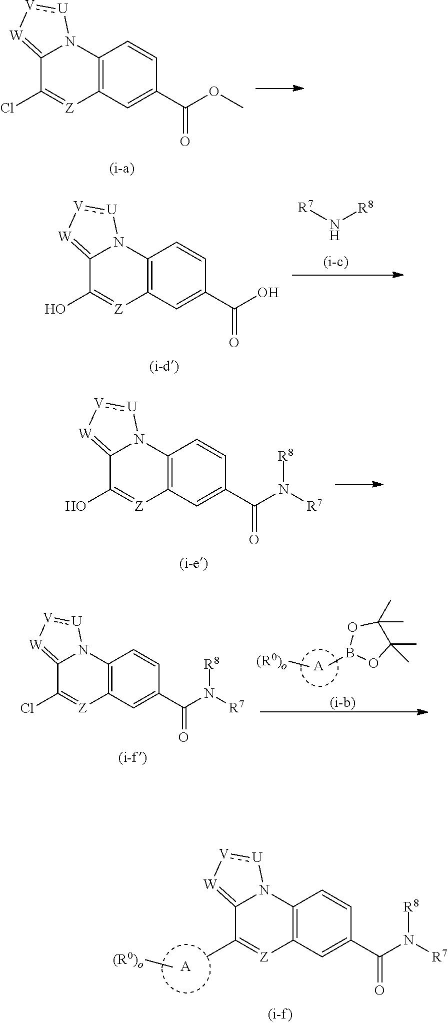

a) the compound represented by formula (i-a) or a pharmaceutically acceptable salt thereof, such as the hydrochloride salt, is hydrolyzed to obtain the compound represented by formula (i-d′);
b) the compound represented by formula (i-d′) is subjected to a condensation reaction with the compound represented by formula (i-c) to obtain the compound represented by formula (i-e′);
c) the compound represented by formula (i-e′) undergoes chlorination to obtain the compound represented by formula (i-f);
d) the compound represented by formula (i-f) is subjected to a condensation reaction with the compound represented by formula (i-b) to obtain the compound represented by formula (i-f). In the method I described above, W is selected from N or CR^{2c}, U and V are each independently selected from N, CR², CR³R⁴, and NR⁵, and the ring A, R⁰, o, Z, [symbol] , R⁷, R⁸, R^{2c}, R², R³, R⁴, and R⁵ are the same as defined above in the present application. In certain specific embodiments, for example, the ring A can be selected from

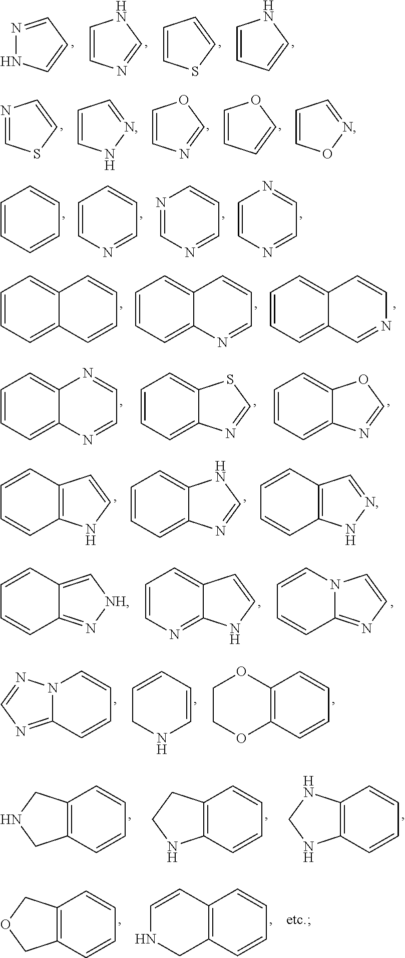

R⁰ can each be independently selected from deuterium, tritium, nitro, hydroxyl, mercapto, chloro, bromo, iodo, fluoro, cyano, ═O, —NH₂, —N(CH₃)₂, —C(O)OCH₂CH₃, carboxyl, —COCH₃, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, piperazinyl, N-methylpiperazinyl, morpholinyl, piperidinyl, cyclopropoxy, cyclopropylmethoxy, cyclopropyl, cyclobutyl, cyclopentyl, cyclohexyl, etc.; o can be selected from 0, 1, or 2; Z can be selected from N, CH, etc.; W can be selected from N, CH, etc.; when [symbol] is a double bond, U and V can each be independently selected from N, CH, CCH₃, CCH₂CH₃, CCF₃, CC(CH₃)₃, CCH(CH₃)₂, CCH₂CH(CH₃)₂, etc.; when [symbol] is a single bond, U and V can each be independently selected from CH₂, NH, C(CH₂)₂, C(CH₃)₂, CHCH₃, CHCH₂CH₃, CHCF₃, CHC(CH₃)₃, CHCH(CH₃)₂, CHCH₂CH(CH₃)₂, C═O, etc.; R⁷ and R⁸ can be selected from methyl, ethyl, n-propyl, isopropyl, n-butyl, isobutyl, tert-butyl, etc., or R⁷ and R⁸, together with the nitrogen atom to which they are attached, can form pyrrolidinyl, piperidinyl, morpholinyl, 4-fluoropiperidinyl, 4,4-difluoropiperidinyl, etc.

The compounds represented by the aforementioned formula (i-a), formula (i-b), and formula (i-c) can be commercially available products or can be prepared by the skilled in the art through the preparation methods disclosed in the prior art. For example, formula (i-a) can be prepared through the following Method i-a1, Method i-a2, Method i-a3, etc.

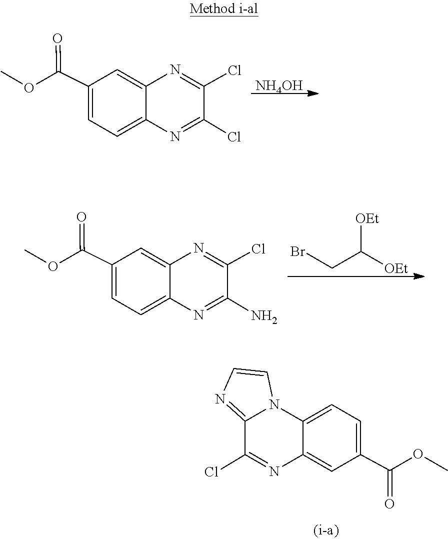

methyl 2,3-dichloroquinoxaline-6-carboxylate is subjected to an ammoniation reaction with ammonium hydroxide, followed by a ring closure reaction to obtain the compound represented by formula (i-a);

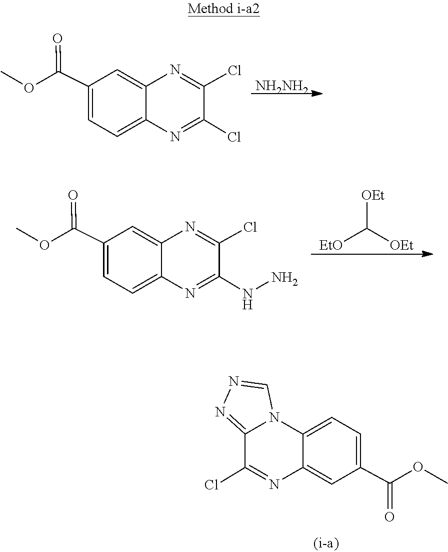

methyl 2,3-dichloroquinoxaline-6-carboxylate is subjected to a reaction with hydrazine, followed by a ring closure reaction to obtain the compound represented by formula (i-a);

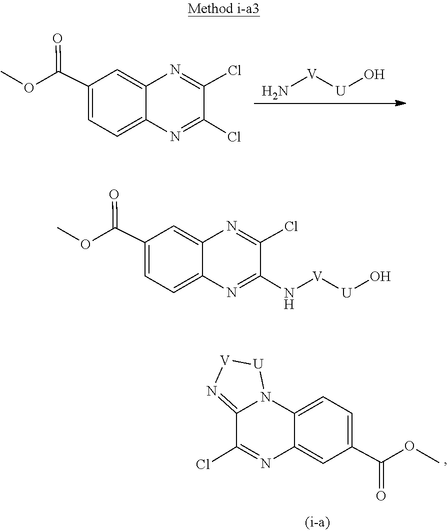

methyl 2,3-dichloroquinoxaline-6-carboxylate is subjected to a condensation reaction with H₂N—V—U—OH, followed by a ring closure reaction to obtain the compound represented by formula (i-a), wherein the U and V are each independently selected from CR³R⁴, NR⁵, and the R³, R⁴, and R⁵ are the same as defined above in the present application, for example, the U and V are each independently selected from NH, CH₂, C(CH₂)₂, C(CH₃)₂, CHCH₃, CHCH₂CH₃, CHCF₃, CHC(CH₃)₃, CHCH(CH₃)₂, CHCH₂CH(CH₃)₂, C—O, etc.

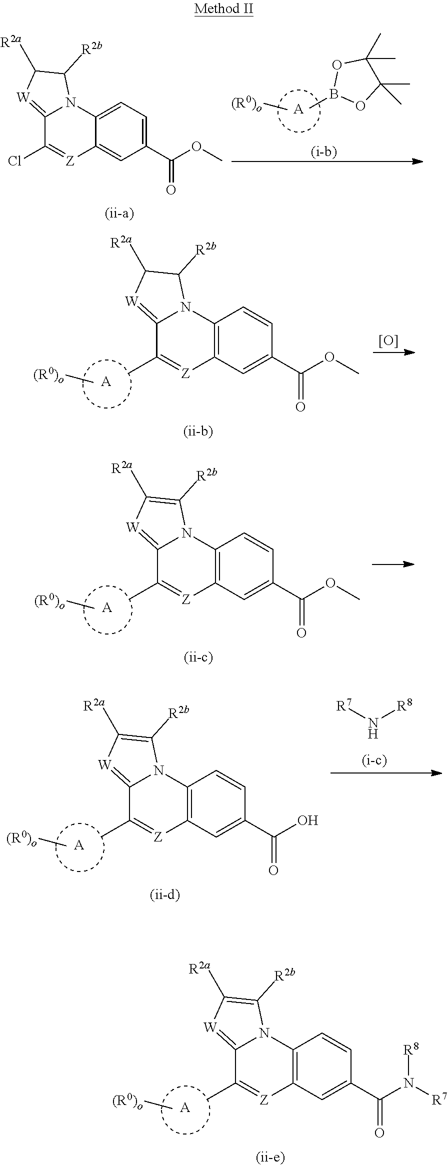

The steps are as follows:
a) the compound represented by formula (ii-a) or a pharmaceutically acceptable salt thereof, such as the hydrochloride salt, is subjected to a reaction with the compound represented by formula (i-b) to obtain the compound represented by formula (ii-b);
b) the compound represented by formula (ii-b) is oxidized to obtain the compound represented by formula (ii-c);
c) the compound represented by formula (ii-c) is hydrolyzed to obtain the compound represented by formula (ii-d);
d) the compound represented by formula (ii-d) is subjected to a condensation reaction with the compound represented by formula (i-c) to obtain the compound represented by formula (ii-e); wherein, W is selected from N or CR^{2c}, and the ring A, R⁰, o, Z, R⁷, R⁸, R^{2c}, R^{2a}, and R^{2b} are the same as defined above in the present application. In certain specific embodiments, for example, the ring A can be selected from

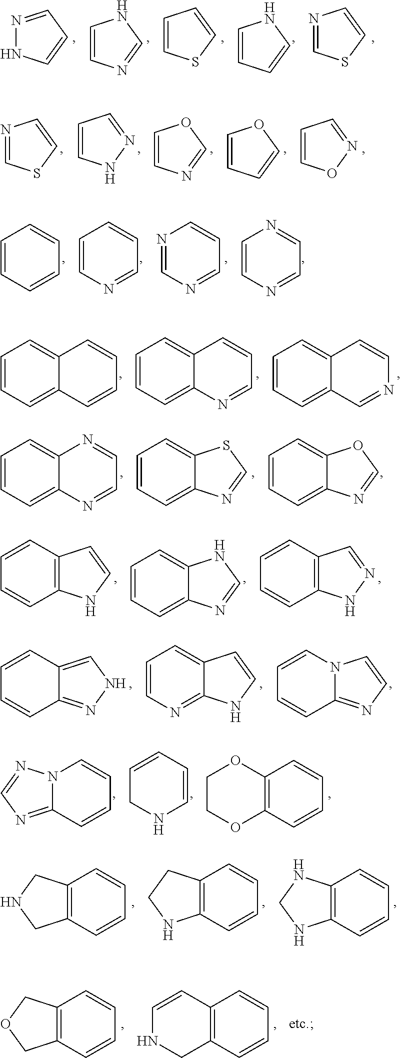

R⁰ can each be independently selected from deuterium, tritium, nitro, hydroxyl, mercapto, chloro, bromo, iodo, fluoro, cyano, ═O, —NH₂, —N(CH₃)₂, —C(O)OCH₂CH₃, carboxyl, —COCH₃, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, piperazinyl, N-methylpiperazinyl, morpholinyl, piperidinyl, cyclopropoxy, cyclopropylmethoxy, cyclopropyl, cyclobutyl, cyclopentyl, cyclohexyl, etc.; o can be selected from 0, 1, or 2; Z can be selected from N, CH; W can be selected from N, CH; R^{2a} and R^{2b} cab each be independently selected from hydrogen, deuterium, tritium, nitro, hydroxyl, mercapto, fluoro, chloro, bromo, iodo, cyano, —NH₂, carboxyl, —CONH₂, —NHCOOH, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, cyclohexyl, etc.; R⁷ and R⁸ can each be independently selected from methyl, ethyl, n-propyl, isopropyl, n-butyl, isobutyl, tert-butyl, etc., or R⁷ and R⁸, together with the nitrogen atom to which they are attached, can form pyrrolidinyl, piperidinyl, morpholinyl, 4-fluoropiperidinyl, 4,4-difluoropiperidinyl, etc.

The compounds represented by the aforementioned formula (ii-a), formula (i-b), and formula (i-c) can be commercially available products or can be prepared by the skilled in the art through the preparation methods disclosed in the prior art. For example, formula (ii-a) can be prepared through a method similar to Method i-a3.

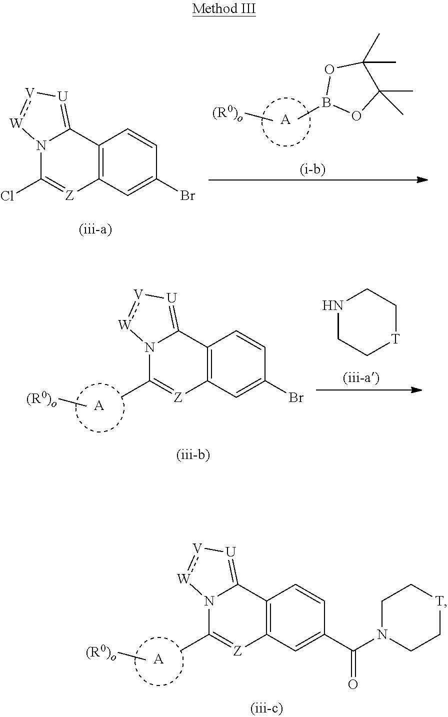

The steps are as follows:
a) the compound represented by formula (iii-a) or a pharmaceutically acceptable salt thereof, such as the hydrochloride salt, is subjected to a reaction with the compound represented by formula (i-b) to obtain the compound represented by formula (iii-b);
b) the compound represented by formula (iii-b) is subjected to a condensation reaction with the compound represented by formula (iii-a′) in CO to obtain the compound represented by formula (iii-c);
wherein, U is selected from N or CR^{2b}, V and W are each independently selected from N, CR², CR³R⁴, and NR⁵, and the ring A, R⁰, o, Z, [symbol] , T, R^{2b}, R², R³, R⁴, and R⁵ are the same as defined above in the present application. In certain specific embodiments, for example, the ring A can be selected from

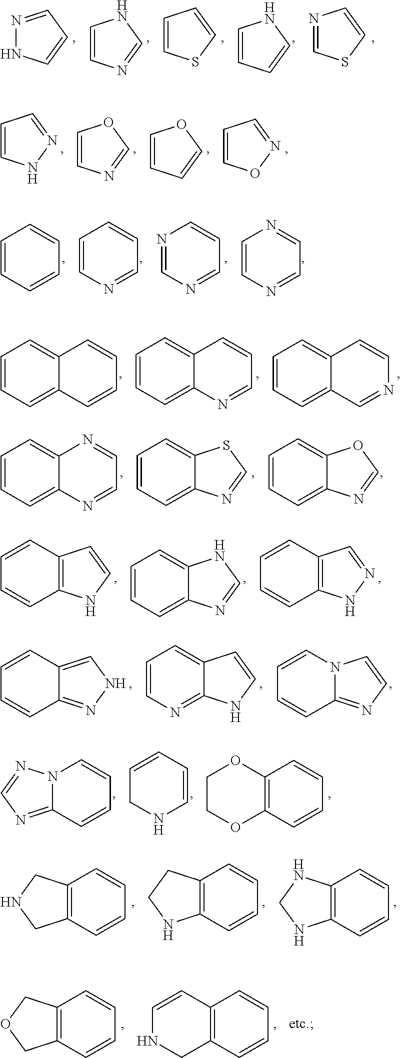

R⁰ can each be independently selected from deuterium, tritium, nitro, hydroxyl, mercapto, chloro, bromo, iodo, fluoro, cyano, ═O, —NH₂, —N(CH₃)₂, —C(O)OCH₂CH₃, carboxyl, —COCH₃, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, piperazinyl, N-methylpiperazinyl, morpholinyl, piperidinyl, cyclopropoxy, cyclopropylmethoxy, cyclopropyl, cyclobutyl, cyclopentyl, cyclohexyl, etc.; o can be selected from 0, 1, or 2; Z can be selected from N, CH; T can be selected from O, a covalent bond, CH₂, CHF, CF₂, C(CH₃)₂, CHCH₃, etc.; U can be selected from N, CH, etc.; when [symbol] is a double bond, V and W can each be independently selected from N, CH, CCH₃, CCH₂CH₃, CCF₃, CC(CH₃)₃, CCH(CH₃)₂, CCH₂CH(CH₃)₂, etc.; when [symbol] is a single bond, V and W can each be independently selected from CH₂, C(CH₂)₂, C(CH₃)₂, CHCH₃, CHCH₂CH₃, CHCF₃, CHC(CH₃)₃, CHCH(CH₃)₂, CHCH₂CH(CH₃)₂, C═O, etc.

The compounds represented by the aforementioned formula (iii-a), formula (i-b), and formula (iii-a′) can be commercially available products or can be prepared by the skilled in the art through the preparation methods disclosed in the prior art. For example, formula (iii-a) can be prepared through the following Method iii-a1, Method iii-a2, Method iii-a3, and Method iii-a4.

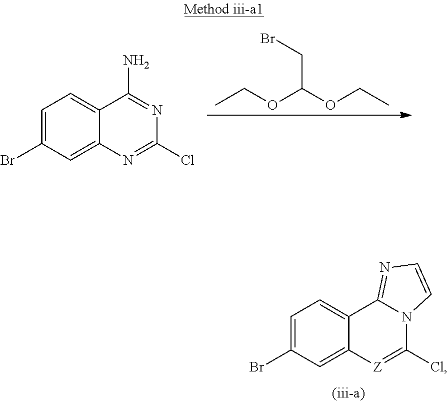

7-bromo-2-chloroquinazolin-4-amine is subjected to a ring closure reaction with bromoacetal to obtain the compound represented by formula (iii-a);

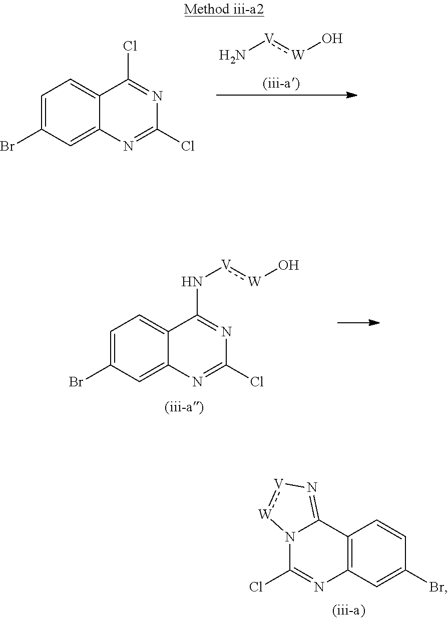

7-bromo-2,4-dichloroquinazoline is subjected to a condensation reaction with the compound represented by formula (iii-a′) to obtain the compound represented by formula (iii-a″), followed by a ring closure reaction to obtain the compound represented by formula (iii-a), wherein V and W are each independently selected from CR³R⁴, NR⁵, and the [symbol] , R³, R⁴, and R⁵ are the same as defined above in the present application. For example, when [symbol] is a double bond, the V and W can each be independently selected from N, CH, CCH₃, CCH₂CH₃, CCF₃, CC(CH₃)₃, CCH(CH₃)₂, CCH₂CH(CH₃)₂, etc.; when [symbol] is a single bond, the V and W can each be independently selected from CH₂, NH, C(CH₂)₂, C(CH₃)₂, CHCH₃, CHCH₂CH₃, CHCF₃, CHC(CH₃)₃, CHCH(CH₃)₂, CHCH₂CH(CH₃)₂, C═O, etc.

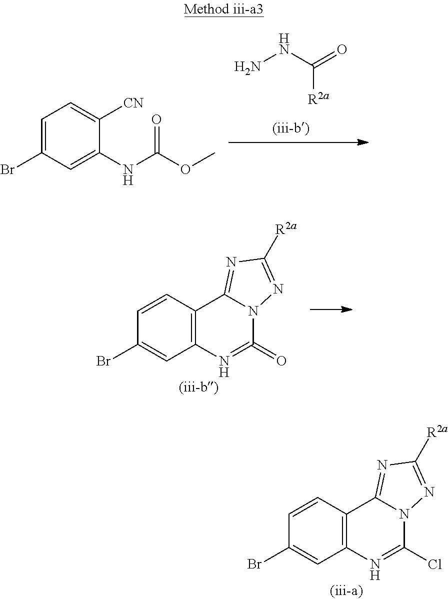

methyl (5-bromo-2-cyanophenyl) carbamate is subjected to a ring closure reaction with the compound represented by formula (iii-b′) to obtain the compound represented by formula (iii-b″), followed by chlorinating with phosphorus oxychloride to obtain the compound represented by formula (iii-a), wherein the R^{2a} is the same as defined above in the present application, for example, the R^{2a} can be selected from hydrogen, deuterium, tritium, CH₃, CH₂CH₃, CF₃, C(CH₃)₃, CH(CH₃)₂, CH₂CH(CH₃)₂, etc.

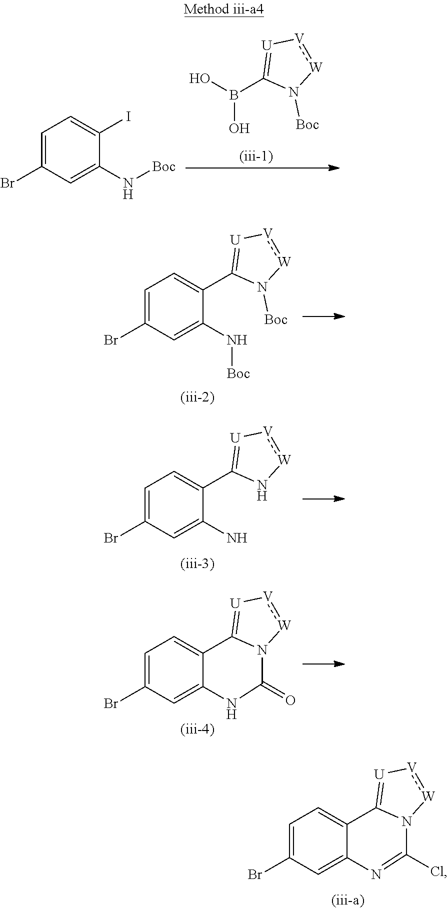

tert-butyl (5-bromo-2-iodophenyl) carbamate is subjected to a condensation reaction with the compound represented by formula (iii-1) to obtain the compound represented by formula (iii-2), followed by a deprotection of Boc to obtain the compound represented by formula (iii-3), then subjected to a ring closure reaction with triphosgene to obtain the compound represented by formula (iii-4), and finally by chlorinating with phosphorus oxychloride to obtain the compound represented by formula (iii-a), wherein U is selected from N or CR^{2b}, V and W are each independently selected from N, CR², CR³R⁴, and NR⁵, and the ring A, R⁰, o, Z, [symbol] , T, R^{2b}, R², R³, R⁴, and R⁵ are consistent with the definitions of the present application as described above. For example, U can be selected from N, CH, etc.; when [symbol] is a double bond, V and W can each be independently selected from N, CH, CCH₃, CCH₂CH₃, CCF₃, CC(CH₃)₃, CCH(CH₃)₂, CCH₂CH(CH₃)₂, etc.; when [symbol] is a single bond, V and W can each be independently selected from CH₂, NH, C(CH₂)₂, C(CH₃)₂, CHCH₃, CHCH₂CH₃, CHCF₃, CHC(CH₃)₃, CHCH(CH₃)₂, CHCH₂CH(CH₃)₂, C═O, etc.

The methods of synthesizing the compounds and intermediates of the present application are described below by way of example. The following examples are only intended to serve as examples of the present application, and should not be taken as a limitation to the scope of the present application. Unless otherwise indicated, the raw materials and reagents involved in the present application are all available commercially, and the specific source does not affect the implementation of the technical solution of the present application.

### Preparation Example 1: Preparation of methyl 4-chloroimidazo[1,2-a]quinoxaline-7-carboxylate

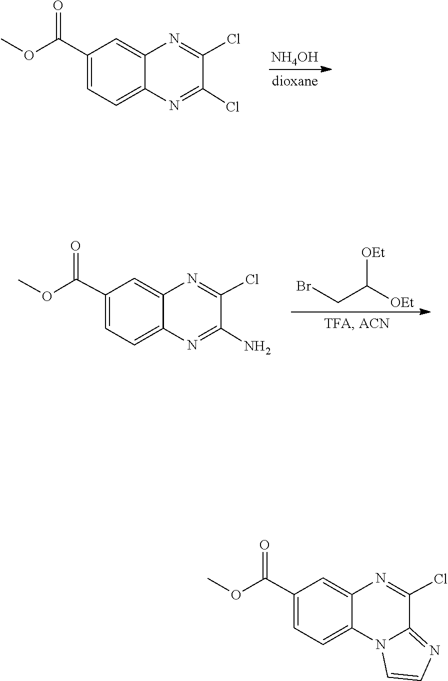 [SMILES: COC(=O)c1ccc2nc(Cl)c(Cl)nc2c1 ; "NH4OH" ; COC(=O)c1ccc2nc(N)c(Cl)nc2c1 ; CCOC(CBr)OCC ; COC(=O)c1ccc2c(c1)nc(Cl)c1nccn12]

### Step 1: Preparation of methyl 2-amino-3-chloroquinoxaline-6-carboxylate

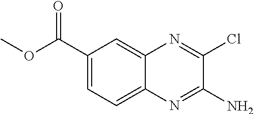 [SMILES: COC(=O)c1ccc2nc(N)c(Cl)nc2c1]

Methyl 2,3-dichloroquinoxaline-6-carboxylate (1.1 g) was dissolved in dioxane (12 mL), into which ammonia water (2 mL) was added to react at 60° C. under a sealed condition for 3 hours. After the disappearance of the raw materials as monitored by LCMS, the reaction system was concentrated under reduced pressure after the reaction was quenched by adding water, and extracted with ethyl acetate for 3 times. The organic phases were combined, dried over anhydrous sodium sulfate, filtered, and concentrated under reduced pressure. The residue was purified by a silica gel column chromatography to obtain 0.86 g of the target compound. MS (ESI) m/z (M+H)⁺=238.0.

### Step 2: Preparation of methyl 4-chloroimidazo[1,2-a]quinoxaline-7-carboxylate

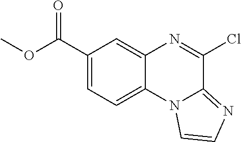 [SMILES: COC(=O)c1ccc2c(c1)nc(Cl)c1nccn12]

Methyl 2-amino-3-chloroquinoxaline-6-carboxylate (0.86 g) was dissolved in acetonitrile (12 mL), into which bromoacetaldehyde diethyl acetal (1.3 g) and trifluoroacetic acid (2 mL) were added to react at 80° C. for 4 hours. After the disappearance of the raw materials as monitored by LCMS, the reaction system was concentrated under reduced pressure, and adjusted to alkaline with a saturated sodium bicarbonate solution. The residue was extracted with ethyl acetate for 3 times. The organic phases were combined, dried over anhydrous sodium sulfate, filtered, and concentrated under reduced pressure. The residue was purified by a silica gel column chromatography to obtain 0.57 g of the target compound. MS (ESI) m/z (M+H)⁺=262.0.

### Preparation Example 2: Preparation of methyl 4′-chloro-1′H-spiro[cyclopropane-1,2′-imidazo[1,2-a]quinoxaline]-7′-carboxylate

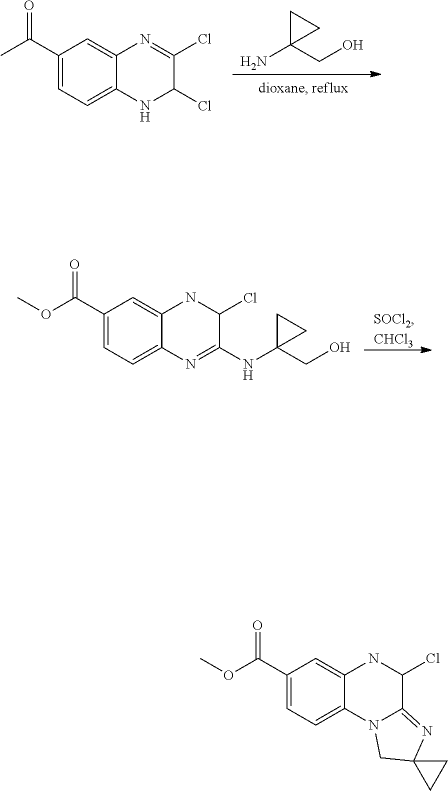 [SMILES: CC(=O)c1ccc2c(c1)N=C(Cl)C(Cl)N2 ; NC1(CO)CC1 ; COC(=O)c1ccc2c(c1)NC(Cl)C(NC1(CO)CC1)=N2 ; COC(=O)c1ccc2c(c1)NC(Cl)C1=NC3(CC3)CN12]

### Step 1: Preparation of methyl 3-chloro-2-(1-(hydroxymethyl)cyclopropyl)aminoquinoxaline-6-carboxylate

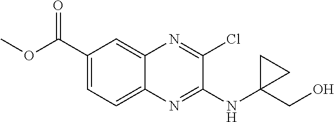 [SMILES: COC(=O)c1ccc2nc(NC3(CO)CC3)c(Cl)nc2c1]

Methyl 2,3-dichloroquinoxaline-6-carboxylate (100 mg) and (1-aminocyclopropyl) methanol (170 mg) were dissolved in dioxane (8 mL), which was then reacted at 80° C. for 2 hours. After the disappearance of the raw materials as monitored by LCMS, the reaction was quenched by adding water, and it was extracted with ethyl acetate for 3 times. The organic phases were combined, dried over anhydrous sodium sulfate, filtered, and concentrated under reduced pressure. The residue was purified by a silica gel column chromatography to obtain 91 mg of the target compound. MS (ESI) m/z (M+H)⁺=308.2.

### Step 2: Preparation methyl 4′-chloro-1′H-spiro[cyclopropane-1,2′-imidazo[1,2-a]quinoxaline]-7′-carboxylate

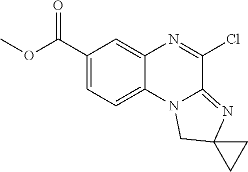 [SMILES: COC(=O)c1ccc2c(c1)N=C(Cl)C1=NC3(CC3)CN12]

Methyl 3-chloro-2-(1-(hydroxymethyl)cyclopropyl)aminoquinoxaline-6-carboxylate (91 mg) was dissolved in chloroform (15 mL), into which thionyl chloride (0.15 mL) was added to react at 70° C. for 2 hours. After the disappearance of the raw materials as monitored by LCMS, the reaction system was concentrated under reduced pressure after the reaction was quenched by adding water, and adjusted to alkaline with a saturated sodium bicarbonate solution, and extracted with ethyl acetate for 3 times. The organic phases were combined, dried over anhydrous sodium sulfate, filtered, and concentrated under reduced pressure. The residue was purified by a silica gel column chromatography to obtain 82 mg of the target compound. MS (ESI) m/z (M+H)⁺=290.1.

### Preparation Example 3: Preparation of methyl 1-methyl-4-chloro-1,2-dihydroimidazo[1,2-a]quinoxaline-7-carboxylate

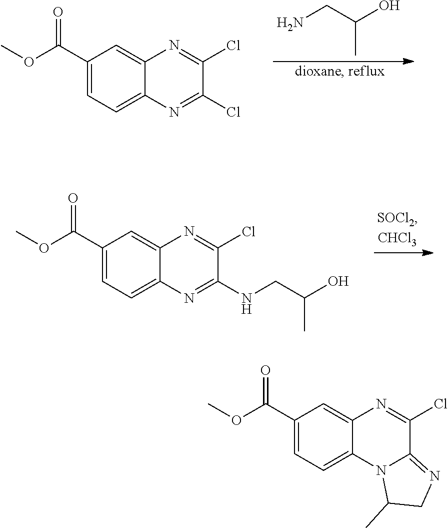

Referring to the preparation method of Preparation Example 2, 103 mg of the title compound was prepared. MS (ESI) m/z (M+H)⁺=278.1.

### Preparation Example 4: Preparation of methyl 2-methyl-4-chloro-1,2-dihydroimidazo[1,2-a]quinoxaline-7-carboxylate

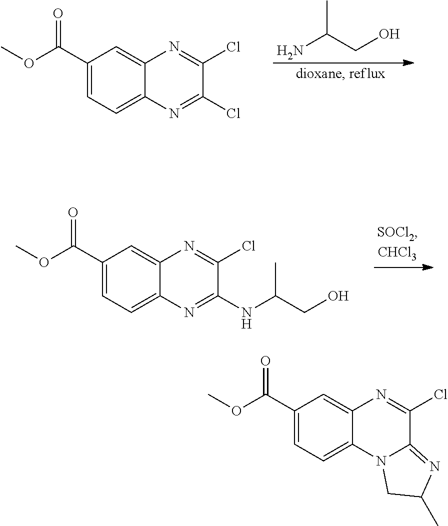

Referring to the preparation method of Preparation Example 2, 98 mg of the title compound was prepared. MS (ESI) m/z (M+H)⁺=278.1.

### Preparation Example 5: Preparation of methyl 4-chloro-1,2-dihydroimidazo[1,2-a]quinoxaline-7-carboxylate

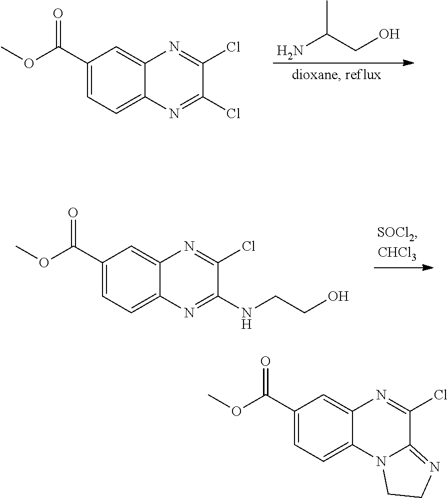

Referring to the preparation method of Preparation Example 2, 110 mg of the title compound was prepared. MS (ESI) m/z (M+H)⁺=264.1.

### Preparation Example 6: Preparation of methyl 2,2-dimethyl-4-chloro-1,2-dihydroimidazo[1,2-a]quinoxaline-7-carboxylate

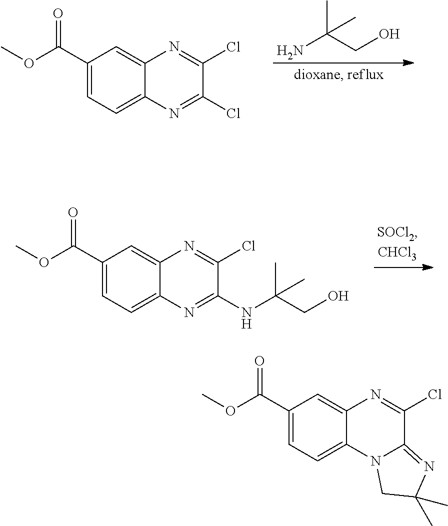

Referring to the preparation method of Preparation Example 2, 120 mg of the title compound was prepared. MS (ESI) m/z (M+H)⁺=292.1.

### Preparation Example 7: Preparation of methyl 4-chloro-[1,2,4]triazolo[4,3-a]quinoxaline-7-carboxylate

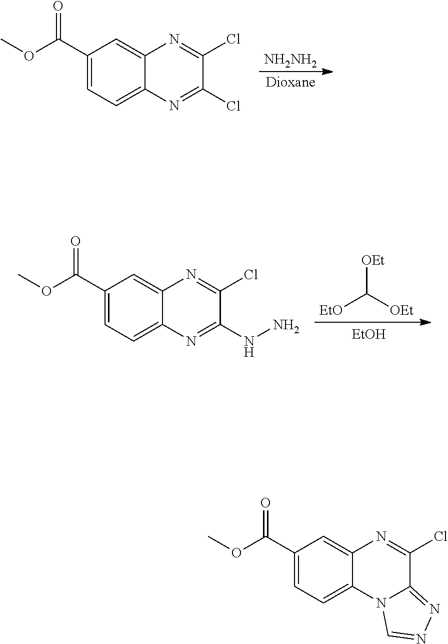

### Step 1: Preparation of methyl 3-chloro-2-hydrazinoquinoxaline-6-carboxylate

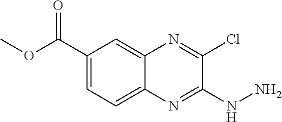 [SMILES: COC(=O)c1ccc2nc(NN)c(Cl)nc2c1]

Methyl 2,3-dichloroquinoxaline-6-carboxylate (300 mg) and hydrazine hydrate (0.57 mL) were dissolved in dioxane (20 mL), which was then reacted at 60° C. for 0.5 hours. After the disappearance of the raw materials as monitored by LCMS, the reaction was quenched by adding water, and it was extracted with ethyl acetate for 3 times. The organic phases were combined, dried over anhydrous sodium sulfate, filtered, and concentrated under reduced pressure. The residue was purified by a silica gel column chromatography to obtain 210 mg of the target compound. MS (ESI) m/z (M+H)⁺=253.2.

### Step 2: Preparation of methyl 4-chloro-[1,2,4]triazolo[4,3-a]quinoxaline-7-carboxylate

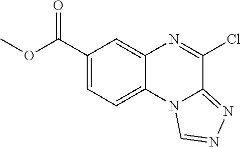 [SMILES: COC(=O)c1ccc2c(c1)nc(Cl)c1nncn12]

Methyl 3-chloro-2-hydrazinoquinoxaline-6-carboxylate (210 mg) and triethyl orthoformate (0.69 mL) were dissolved in ethanol (15 mL), which were then reacted at 80° C. for 2 hours. After the disappearance of the raw materials as monitored by LCMS, the reaction system was concentrated under reduced pressure after the reaction was quenched by adding water, and extracted with ethyl acetate for 3 times. The organic phases were combined, dried over anhydrous sodium sulfate, filtered, and concentrated under reduced pressure. The residue was purified by a silica gel column chromatography to obtain 175 mg of the target compound. MS (ESI) m/z (M+H)⁺=263.0.

### Preparation Example 8: Preparation of methyl 2-isopropyl-4-chloro-1,2-dihydroimidazo[1,2-a]quinoxaline-7-carboxylate

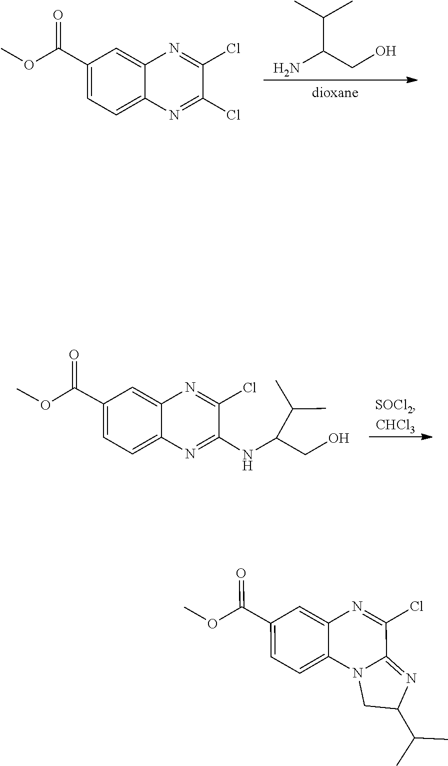

Referring to the preparation method of Preparation Example 2, 210 mg of the title compound was prepared. MS (ESI) m/z (M+H)⁺=306.1.

### Preparation Example 9: Preparation of methyl 2-isopropylmethyl-4-chloro-1,2-dihydroimidazo[1,2-a]quinoxaline-7-carboxylate

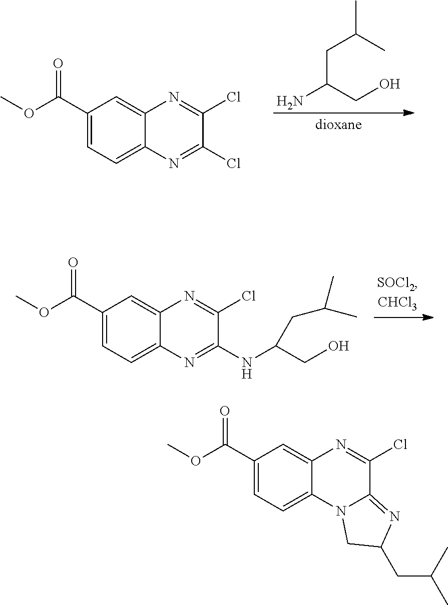

Referring to the preparation method of Preparation Example 2, 280 mg of the title compound was prepared. MS (ESI) m/z (M+H)⁺=320.1.

### Preparation Example 10: Preparation of methyl 2-tert-butyl-4-chloro-1,2-dihydroimidazo[1,2-a]quinoxaline-7-carboxylate

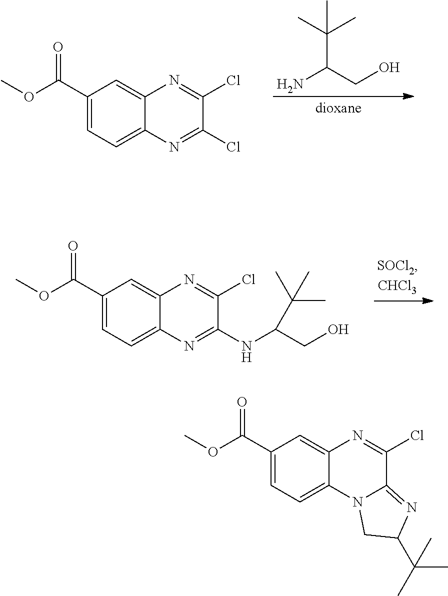

Referring to the preparation method of Preparation Example 2, 280 mg of the title compound was prepared. MS (ESI) m/z (M+H)⁺=320.1.

### Preparation Example 11: Preparation of 8-bromo-5-chloroimidazo[1,2-c]quinazoline

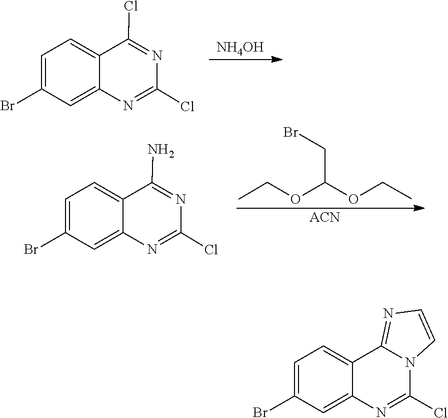

### Step 1: Preparation of 7-bromo-2-chloroquinazolin-4-amine

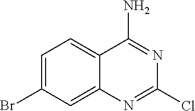 [SMILES: Nc1nc(Cl)nc2cc(Br)ccc12]

7-bromo-2,4-dichloroquinazoline (2.8 g) was weighed and dissolved in 20 mL of acetonitrile, into which 15 mL of ammonia water was added to react at room temperature for 1 h. The reaction was completed as monitored by LCMS, and concentrated under reduced pressure to obtain the title compound (2.8 g). MS (ESI) m/z (M+H)⁺=257.9.

### Step 2: Preparation of 8-bromo-5-chloroimidazo[1,2-c]quinazoline

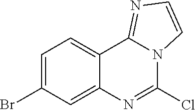 [SMILES: Clc1nc2cc(Br)ccc2c2nccn12]

7-bromo-2-chloroquinazolin-4-amine (1.3 g) was weighed and suspended in 12 mL of acetonitrile, into which bromoacetal (2.7 mL) was added to react under microwave at 120° C. for 2 h. The reaction was completed as monitored by TLC and LCMS, and concentrated under reduced pressure, and purified by a silica gel column chromatography to obtain 492 mg of the title compound. MS (ESI) m/z (M+H)⁺=281.9.

### Preparation Example 12: Preparation of 8-bromo-5-chloro-2,3-dihydroimidazo[1,2-c]quinazoline

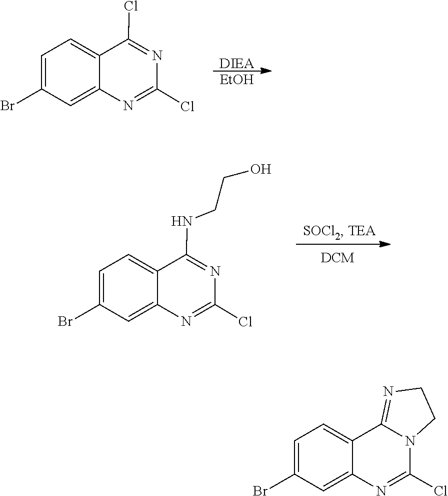 [SMILES: "EtOH" ; Clc1nc(Cl)c2ccc(Br)cc2n1 ; "DIEA" ; OCCNc1nc(Cl)nc2cc(Br)ccc12 ; ClC1=Nc2cc(Br)ccc2C2=NCCN12]

Step 1: Preparation of 2-((7-bromo-2-chloroquinazolin-4-yl)amino) ethanol

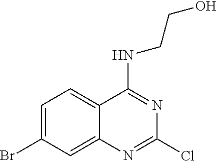 [SMILES: OCCNc1nc(Cl)nc2cc(Br)ccc12]

7-bromo-2,4-dichloroquinazoline (3.0 g) and 2-aminoethanol (659.3 mg) were dissolved in ethanol (60.0 mL), into which N,N-diisopropylethylamine (2.8 g) was then added. After the addition was completed, the reaction system was placed at 80° C. and stirred for 5 hours. LCMS showed that the raw materials were basically completely reacted. After concentration of the reaction solution, the title compound was obtained through purification by a silica gel column chromatography. MS (ESI) m/z (M+H)⁺=302.0.

### Step 2: Preparation of 8-bromo-5-chloro-2,3-dihydroimidazo[1,2-c]quinazoline

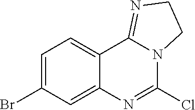 [SMILES: ClC1=Nc2cc(Br)ccc2C2=NCCN12]

2-((7-bromo-2-chloroquinazolin-4-yl)amino) ethanol (800.0 mg) and triethylamine (535.1 mg) were dissolved in dichloromethane (16.0 mL), into which thionyl chloride (629.2 mg) was then added. After the addition was completed, the reaction system was placed at room temperature and stirred for 16 hours. LCMS showed that the raw materials were basically completely reacted. After concentration of the reaction solution, the title compound was obtained through purification by a silica gel column chromatography. MS (ESI) m/z (M+H)⁺=284.0.

### Preparation Example 13: Preparation of 8-bromo-5-chloro-1,2,4-triazolo[1,5-c]quinazoline

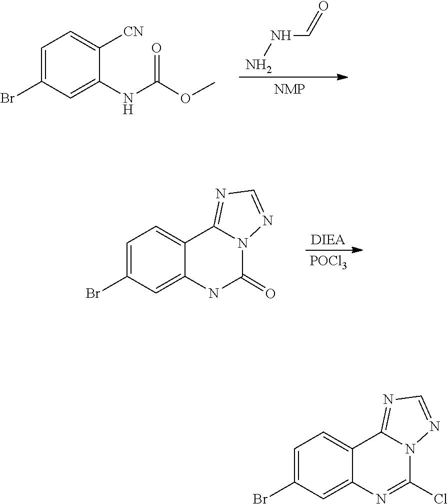

### Step 1: Preparation of 8-bromo-6H-1,2,4-triazolo[1,5-c]quinazolin-5-one

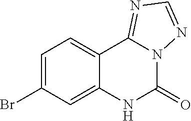 [SMILES: O=c1[nH]c2cc(Br)ccc2c2ncnn12]

Ethyl N-(5-bromo-2-cyano-phenyl) carbamate (5 g) was dissolved in N-methylpyrrolidone (100.0 mL), into which formylhydrazine (1.12 g) was then added. After the addition was completed, the reaction system was heated and stirred at 130° C. for 16 hours. LCMS showed that the raw materials were basically completely reacted. The reaction solution was diluted with water. The aqueous phase was extracted with ethyl acetate. The organic phases were combined and washed with saturated brine, and then dried over anhydrous sodium sulfate. After concentration, the title compound was obtained. MS (ESI) m/z (M+H)⁺=265.0.

### Step 2: Preparation of 8-bromo-5-chloro-1,2,4-triazolo[1,5-c]quinazoline

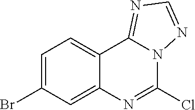 [SMILES: Clc1nc2cc(Br)ccc2c2ncnn12]

8-bromo-6H-1,2,4-triazolo[1,5-c]quinazolin-5-one (3.0 g) was dissolved in phosphorus oxychloride (20 mL), into which DIEA (14.60 g) was then added. The reaction solution was reacted at 110° C. for 16 hours. LCMS showed that the reaction was completed. The reaction solution was concentrated, and water (30 mL) was added to the concentrated crude product. The aqueous phase was extracted with ethyl acetate. The organic phases were combined and washed with saturated brine, and then dried over anhydrous sodium sulfate. After concentration, the title compound was obtained. MS (ESI) m/z (M+H)⁺=282.9.

### Preparation Example 14: Preparation of 8-bromo-5-chloro-2-methyl-1,2,4-triazolo[1,5-c]quinazoline

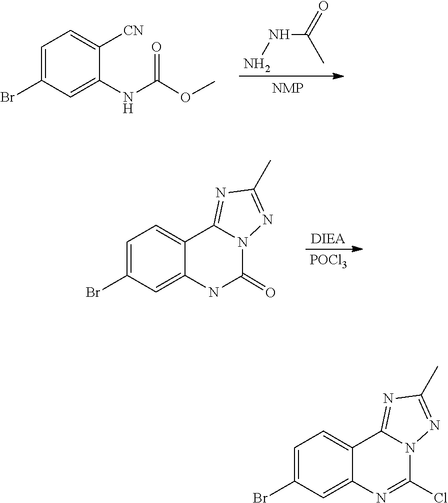

### Step 1: Preparation of 8-bromo-2-methyl-6H-1,2,4-triazolo[1,5-c]quinazolin-5-one

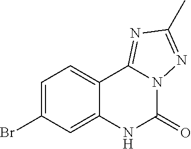 [SMILES: Cc1nc2c3ccc(Br)cc3[nH]c(=O)n2n1]

Ethyl N-(5-bromo-2-cyano-phenyl) carbamate (5.0 g) was dissolved in N-methylpyrrolidone (100.0 mL), into which acethydrazide (1.38 g) was then added. After the addition was completed, the reaction system was heated and stirred at 130° C. for 16 hours. LCMS showed that the raw materials were basically completely reacted. The reaction solution was diluted with water. The aqueous phase was extracted with ethyl acetate. The organic phases were combined and washed with saturated brine, and then dried over anhydrous sodium sulfate. After concentration, the title compound was obtained. MS (ESI) m/z (M+H)⁺=278.9.

### Step 2: Preparation of 8-bromo-5-chloro-2-methyl-1,2,4-triazolo[1,5-c]quinazoline

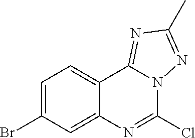 [SMILES: Cc1nc2c3ccc(Br)cc3nc(Cl)n2n1]

8-bromo-2-methyl-6H-1,2,4-triazolo[1,5-c]quinazolin-5-one (3.0 g) was dissolved in phosphorus oxychloride (20 mL), into which DIEA (13.87 g) was then added. The reaction solution was reacted at 110° C. for 16 hours. LCMS showed that the reaction was completed. The reaction solution was concentrated, and water (30 mL) was added to the concentrated crude product. The aqueous phase was extracted with ethyl acetate. The organic phases were combined and washed with saturated brine, then dried over anhydrous sodium sulfate. After concentration, the title compound was obtained. MS (ESI) m/z (M+H)⁺=296.9.

### Preparation Example 15: Preparation of 8-bromo-5-chloro-2-isopropyl-1,2,4-triazolo[1,5-c]quinazoline

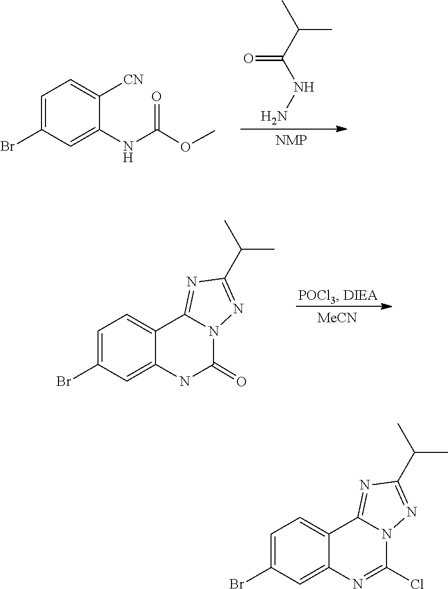

### Step 1: Preparation of 8-bromo-2-isopropyl-6H-1,2,4-triazolo[1,5-c]quinazolin-5-one

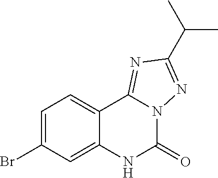 [SMILES: CC(C)c1nc2c3ccc(Br)cc3[nH]c(=O)n2n1]

Ethyl N-(5-bromo-2-cyano-phenyl) carbamate (5.0 g) was dissolved in N-methylpyrrolidone (100.0 mL), into which isobutyric acid hydrazide (3.8 g) was then added. After the addition was completed, the reaction system was heated and stirred at 130° C. for 16 hours. LCMS showed that the raw materials were basically completely reacted. The reaction solution was diluted with water. The aqueous phase was extracted with ethyl acetate. The organic phases were combined and washed with saturated brine, and then dried over anhydrous sodium sulfate. After concentration, the title compound was obtained. MS (ESI) m/z (M+H)⁺=307.0.

### Step 2: Preparation of 8-bromo-5-chloro-2-isopropyl-1,2,4-triazolo[1,5-c]quinazoline

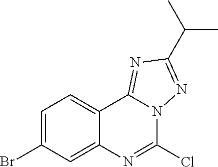 [SMILES: CC(C)c1nc2c3ccc(Br)cc3nc(Cl)n2n1]

8-bromo-2-isopropyl-6H-1,2,4-triazolo[1,5-c]quinazolin-5-one (2.0 g) was dissolved in acetonitrile (20 mL), into which phosphorus oxychloride (4.99 g) and DIEA (4.21 g) were added at 0° C. The reaction solution was reacted at 110° C. for 16 hours. LCMS showed that the reaction was completed. The reaction solution was concentrated, and water (30 mL) was added to the concentrated crude product. The aqueous phase was extracted with ethyl acetate. The organic phases were combined and washed with saturated brine, and then dried over anhydrous sodium sulfate. After concentration, the title compound was obtained. MS (ESI) m/z (M+H)⁺=325.0.

### Preparation Example 16: Preparation of 8-bromo-5-chloro-2-trifluoromethyl-1,2,4-triazolo[1,5-c]quinazoline

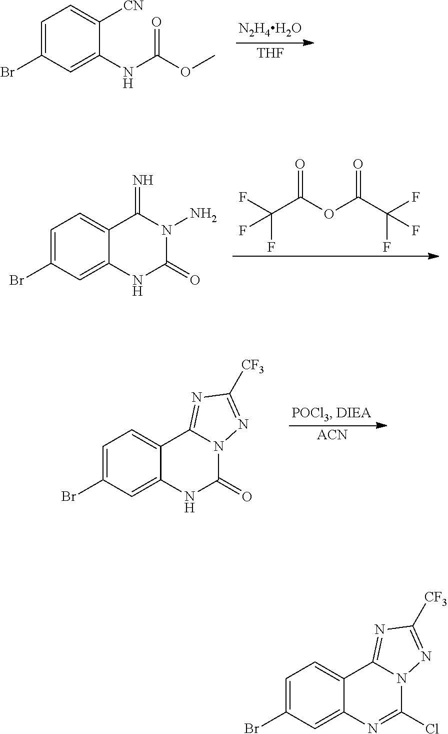

### Step 1: Preparation of 3-amino-7-bromo-4-imino-3,4-dihydroquinazolin-2 (1H)-one

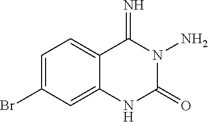 [SMILES: N=c1c2ccc(Br)cc2[nH]c(=O)n1N]

Methyl (5-bromo-2-cyanophenyl) carbamate (3.2 g) was dissolved in tetrahydrofuran (60.0 mL), into which hydrazine hydrate (1.57 g) was then added. After the addition was completed, the reaction system was placed at 60° C. and stirred for 16 hours. The white solids precipitated out. LCMS showed that the raw materials were completely reacted. The reaction solution was naturally cooled, filtered, and dried to obtain the title compound. MS (ESI) m/z (M+H)⁺=255.2.

### Step 2: Preparation of 8-bromo-6H-2-trifluoromethyl-1,2,4-triazolo[1,5-c]quinazolin-5-one

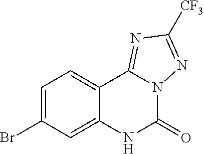 [SMILES: O=c1[nH]c2cc(Br)ccc2c2nc(C(F)(F)F)nn12]

3-amino-7-bromo-4-imino-3,4-dihydroquinazolin-2 (1H)-one (2.0 g) was dissolved in trifluoroacetic anhydride (60 mL), and the reaction solution was stirred at 80° C. for 16 hours. LCMS showed that the reaction was completed. The reaction solution was concentrated to obtain the title compound. MS (ESI) m/z (M+H)⁺=333.1.

### Step 3: Preparation of 8-bromo-5-chloro-2-trifluoromethyl-1,2,4-triazolo[1,5-c]quinazoline

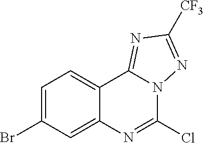 [SMILES: FC(F)(F)c1nc2c3ccc(Br)cc3nc(Cl)n2n1]

8-bromo-6H-2-trifluoromethyl-1,2,4-triazolo[1,5-c]quinazolin-5-one (0.5 g) was dissolved in acetonitrile (100.0 mL), into which diisopropylethylamine (967.5 mg) and phosphorus oxychloride (1.1 g) were sequentially added to react with stirring at 100° C. for 16 hours. LCMS showed that the reaction was completed. The reaction solution was quenched by adding water. The aqueous phase was extracted with ethyl acetate. The organic phases were combined and washed with saturated brine, then dried over anhydrous sodium sulfate. After concentration, the title compound was obtained through purification by a silica gel column chromatography. MS (ESI) m/z (M+H)⁺=351.2.

### Preparation Example 17: Preparation of 8-bromo-5-chloro-pyrrolo[1,2-c]quinazoline

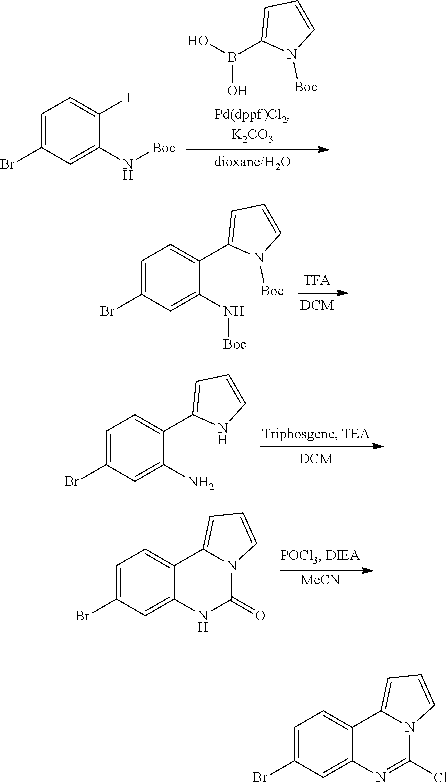

### Step 1: Preparation of tert-butyl 2-(4-bromo-2-(tert-butyloxycarbonylamino)phenyl) pyrrole-1-carboxylate

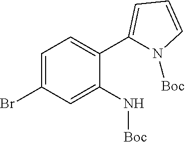 [SMILES: CC(C)(C)OC(=O)Nc1cc(Br)ccc1-c1cccn1C(=O)OC(C)(C)C]

Tert-butyl N-(5-bromo-2-iodo-phenyl) carbamate (1.5 g) and (1-tert-butyloxycarbonylpyrrol-2-yl) boronic acid (795.24 mg) were dissolved in a mixed solvent of dioxane (15 mL) and water (1.5 mL), into which potassium carbonate (1.56 g) and [1,1′-bis(diphenylphosphino)ferrocene]dichloropalladium (307.51 mg) were then added. After the addition was completed, the reaction was stirred at 90° C. under nitrogen for 4 hours. LCMS showed that the raw materials were basically completely reacted. The reaction solution was diluted with water. The aqueous phase was extracted with ethyl acetate. The organic phases were combined and washed with saturated brine, and then dried over anhydrous sodium sulfate. After concentration, the title compound was obtained through purification by a silica gel column chromatography.

MS (ESI) m/z (M+H)⁺=437.1.

### Step 2: Preparation of 5-bromo-2-(1H-pyrrol-2-yl) aniline

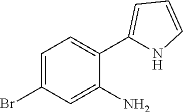 [SMILES: Nc1cc(Br)ccc1-c1ccc[nH]1]

Tert-butyl 2-(4-bromo-2-(tert-butyloxycarbonylamino)phenyl) pyrrole-1-carboxylate (1.3 g) was dissolved in dichloromethane (10 mL) and trifluoroacetic acid (1 mL). The reaction solution was stirred at 25° C. for 3 hours. LCMS showed that the raw materials were basically completely reacted. The reaction mixture was concentrated to obtain the title compound. MS (ESI) m/z (M+H)⁺=237.0.

### Step 3: Preparation of 8-bromo-6H-pyrrolo[1,2-c]quinazolin-5-one

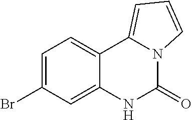 [SMILES: O=c1[nH]c2cc(Br)ccc2c2cccn12]

5-bromo-2-(1H-pyrrol-2-yl) aniline (700 mg) was dissolved in ultra-dry dichloromethane (15 mL) at 25° C., into which triethylamine (1.5 g) and triphosgene (438.06 mg) were added. The reaction solution was stirred at 25° C. for another 3 hours. LCMS showed that the reaction was completed. The reaction solution was concentrated and purified by a silica gel column chromatography to obtain the title compound. MS (ESI) m/z (M+H)⁺=263.0.

### Step 4: Preparation of 8-bromo-5-chloro-pyrrolo[1,2-c]quinazoline

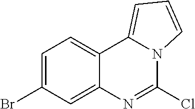 [SMILES: Clc1nc2cc(Br)ccc2c2cccn12]

8-bromo-6H-pyrrolo[1,2-c]quinazolin-5-one (600 mg) was dissolved in phosphorus oxychloride (10 mL), into which N,N-diisopropylethylamine (2.94 g) was added at 25° C. After the addition, the reaction solution was stirred at 110° C. for 3 hours. LCMS showed that the raw materials were basically completely reacted. The reaction solution was concentrated and purified by a silica gel column chromatography to obtain the title compound. MS (ESI) m/z (M+H)⁺=280.9.

### Preparation Example 18: Preparation of 8-bromo-5-chloro-pyrazolo[1,5-c]quinazoline

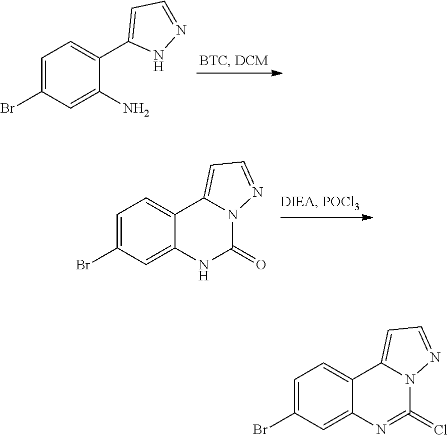

### Step 1: Preparation of 8-bromo-6H-pyrazolo[1,5-c]quinazolin-5-one

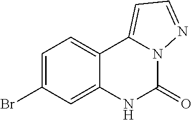 [SMILES: O=c1[nH]c2cc(Br)ccc2c2ccnn12]

Triphosgene (0.4 g) was added to a solution of 5-bromo-2-(1H-pyrazol-5-yl) aniline (0.3 g) in dichloromethane (6 mL), and the resulting mixed system was stirred at room temperature overnight. The completion of the reaction was monitored by LCMS. The reaction solution was diluted with water and extracted with dichloromethane. The organic phases were combined and washed with saturated brine, and then dried over anhydrous sodium sulfate. The reaction solution was concentrated and purified by a silica gel column chromatography to obtain the title compound. MS (ESI) m/z (M+H)⁺=265.0.

### Step 2: Preparation of 8-bromo-5-chloropyrazolo[1,5-c]quinazoline

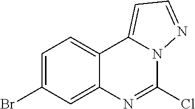 [SMILES: Clc1nc2cc(Br)ccc2c2ccnn12]

8-bromo-6H-pyrazolo[1,5-c]quinazolin-5-one (0.28 g) was dissolved in phosphorus oxychloride (5 mL) in a 50 mL single-neck flask, into which N,N-diisopropylethylamine (1.3 g) was then added. The reaction solution was stirred at 110° C. overnight. LCMS showed that the raw materials were completely reacted. A rotary evaporation was performed on the reaction solution to dryness under pressure. After the removal of phosphorus oxychloride via the rotary evaporation, ethyl acetate was added to dilute the remains, and it was washed with water. The organic phases were combined and washed with saturated brine, and then dried over anhydrous sodium sulfate. The title compound was obtained through separating and purifying by a silica gel column chromatography. MS (ESI) m/z (M+H)⁺=281.9.

### Preparation Example 19: Preparation of 9-bromo-5-chloroimidazo[1,2-c]quinazoline

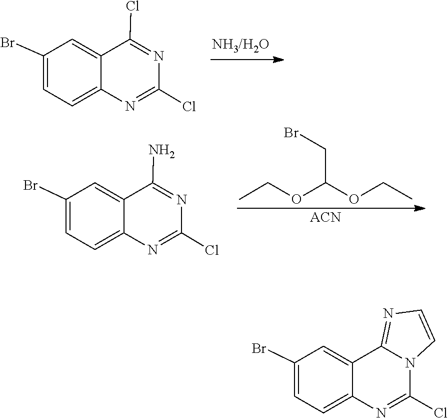

### Step 1: Preparation of 6-bromo-2-chloroquinazolin-4-amine

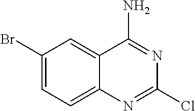 [SMILES: Nc1nc(Cl)nc2ccc(Br)cc12]

6-bromo-2,4-dichloroquinazoline (2.8 g) was weighed and dissolved in 20 mL of acetonitrile, into which 15 mL of ammonia water was added to react at room temperature for 1 h. The reaction was completed as monitored by LCMS, and concentrated under reduced pressure to obtain the title compound (2.3 g). MS (ESI) m/z (M+H)⁺=258.0.

### Step 2: Preparation of 9-bromo-5-chloroimidazo[1,2-c]quinazoline

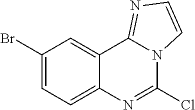 [SMILES: Clc1nc2ccc(Br)cc2c2nccn12]

6-bromo-2-chloroquinazolin-4-amine (1.0 g) was weighed and suspended in 12 mL of acetonitrile, into which bromoacetal (2.7 mL) was added to react under microwave at 120° C. for 2 h. The reaction was completed as monitored by TLC and LCMS, and concentrated under reduced pressure, and purified by a silica gel column chromatography to obtain 707 mg of the title compound. MS (ESI) m/z (M+H)⁺=281.9; MS (ESI) m/z (M+H)⁺=282.0.

### Example 1: Preparation of (4-phenylimidazo[1,2-a]quinoxalin-7-yl)(piperidin-1-yl)methanone

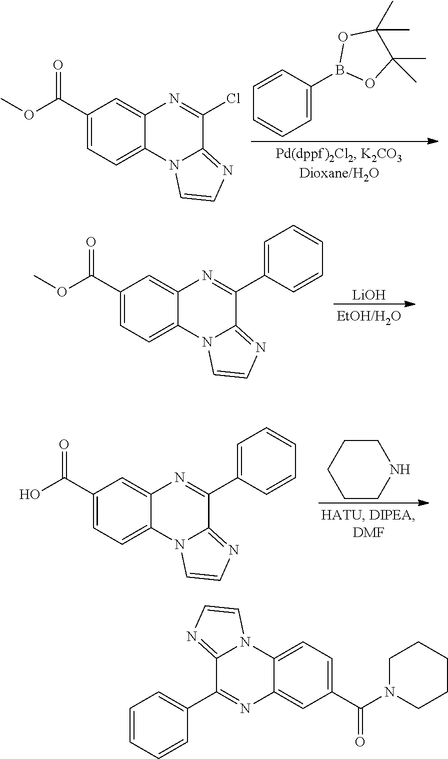

### Step 1: Preparation of methyl 4-phenylimidazo[1,2-a]quinoxaline-7-carboxylate

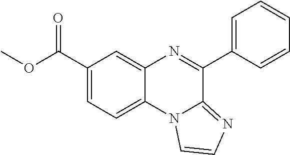 [SMILES: COC(=O)c1ccc2c(c1)nc(-c1ccccc1)c1nccn12]

Methyl 4-chloroimidazo[1,2-a]quinoxaline-7-carboxylate (100 mg), 4,4,5,5-tetramethyl-2-phenyl-1,3,2-dioxaborolane (155 mg), [1,1′-bis(diphenylphosphino)ferrocene]dichloropalladium (32 mg) and potassium carbonate (157 mg) were dissolved in a mixed solution (5 mL) of dioxane and water and reacted at 85° C. under nitrogen protection for 0.5 hours. After the disappearance of the raw materials as monitored by LCMS, the reaction was quenched by adding water and extracted with ethyl acetate for 3 times. The organic phases were combined, dried over anhydrous sodium sulfate, filtered, and concentrated under reduced pressure. The residue was purified by a silica gel column chromatography to obtain 70 mg of the target compound. MS (ESI) m/z (M+H)⁺=304.

### Step 2: Preparation of 4-phenylimidazo[1,2-a]quinoxaline-7-carboxylic acid

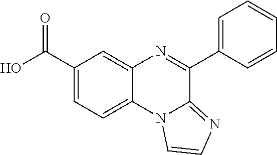 [SMILES: O=C(O)c1ccc2c(c1)nc(-c1ccccc1)c1nccn12]

Methyl 4-phenylimidazo[1,2-a]quinoxaline-7-carboxylate (70 mg) and lithium hydroxide (50 mg) were dissolved in a mixed solution (8 mL) of ethanol and water and reacted at 85° C. for 0.5 hours. After the disappearance of the raw materials as monitored by LCMS, the reaction system was concentrated under reduced pressure to obtain 120 mg of the crude target compound. MS (ESI) m/z (M+H)⁺=290.0.

### Step 3: Preparation of (4-phenylimidazo[1,2-a]quinoxalin-7-yl)(piperidin-1-yl)methanone

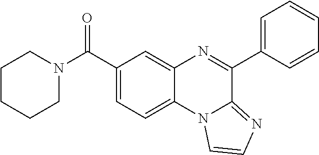 [SMILES: O=C(c1ccc2c(c1)nc(-c1ccccc1)c1nccn12)N1CCCCC1]

4-phenylimidazo[1,2-a]quinoxaline-7-carboxylic acid (120 mg), piperidine (0.1 mL), HATU (315 mg), and DIPEA (0.5 mL) were dissolved in DMF solution (8 mL) and reacted at room temperature for 1 hour. After the disappearance of the raw materials as monitored by LCMS, the reaction was quenched by adding water and extracted with ethyl acetate for 3 times. The organic phases were combined, dried over anhydrous sodium sulfate, filtered, and concentrated under reduced pressure. The residue was purified by a silica gel column chromatography to obtain 23 mg of the target compound. MS (ESI) m/z (M+H)⁺=357.1. ¹H NMR (400 MHz, DMSO-d₆) δ 8.89 (s, 1H), 8.74 (dd, J=6.7, 3.0 Hz, 2H), 8.42 (d, J=8.5 Hz, 1H), 8.05-7.98 (m, 1H), 7.89 (s, 1H), 7.72-7.66 (m, 1H), 7.57-7.51 (m, 3H), 3.58 (s, 4H), 1.63-1.43 (m, 6H).

### Example 2: Preparation of (4-(benzo[d]oxazol-5-yl) imidazo[1,2-a]quinoxalin-7-yl)(piperidin-1-yl)methanone

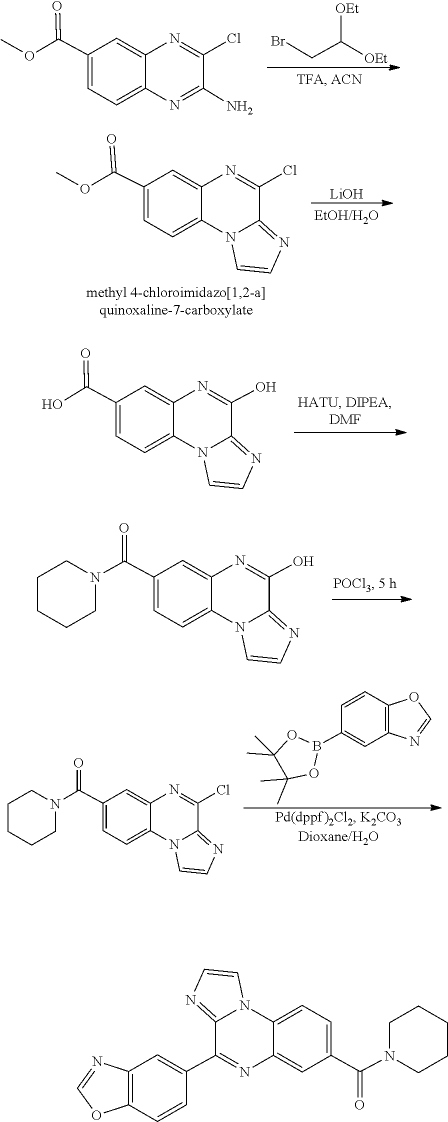

### Step 1: Preparation of 4-hydroxyimidazo[1,2-a]quinoxaline-7-carboxylic acid

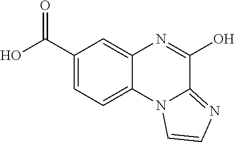 [SMILES: O=C(O)c1ccc2c(c1)nc(O)c1nccn12]

Methyl 4-chloroimidazo[1,2-a]quinoxaline-7-carboxylate (1.3 g) was dissolved in a mixed solution (15 mL) of ethanol and water, into which lithium hydroxide (0.45 g) was added to react in an oil bath at 80° C. for 0.5 hours. After the disappearance of the raw materials as monitored by LCMS, the solvent was partially concentrated under reduced pressure and purified by a reverse phase preparative chromatography to obtain 1.1 g of the target compound. MS (ESI) m/z (M+H)⁺=230.0.

### Step 2: Preparation of (4-hydroxyimidazo[1,2-a]quinoxalin-7-yl)(piperidin-1-yl)methanone

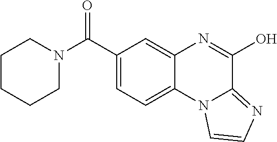 [SMILES: O=C(c1ccc2c(c1)nc(O)c1nccn12)N1CCCCC1]

4-hydroxyimidazo[1,2-a]quinoxaline-7-carboxylic acid (1.1 g) and HATU (2.7 g) were dissolved in DMF (15 mL), into which DIPEA (1.86 g) was added to react with stirring at room temperature for 1 hour. After the disappearance of the raw materials as monitored by LCMS, the reaction was quenched by adding water and extracted with ethyl acetate for 3 times. The organic phases were combined, dried over anhydrous sodium sulfate, filtered, and concentrated under reduced pressure. The residue was purified by a silica gel column chromatography to obtain 1.1 g of the target compound. MS (ESI) m/z (M+H)⁺=297.0.

### Step 3: Preparation of (4-chloroimidazo[1,2-a]quinoxalin-7-yl)(piperidin-1-yl)methanone

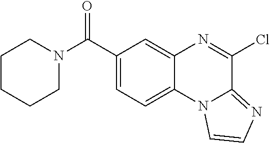 [SMILES: O=C(c1ccc2c(c1)nc(Cl)c1nccn12)N1CCCCC1]

(4-hydroxyimidazo[1,2-a]quinoxalin-7-yl)(piperidin-1-yl)methanone (1.1 g, 3.72 mmol) was dissolved in phosphorus oxychloride (25 mL), which were reacted in an oil bath with stirring at 80° C. for 5 hours. After the disappearance of the raw materials as monitored by LCMS, phosphorus oxychloride was removed by concentration under reduced pressure, and adjusted to alkaline with a saturated sodium bicarbonate solution. The residue was extracted with ethyl acetate for 3 times. The organic phases were combined, dried over anhydrous sodium sulfate, filtered, and concentrated under reduced pressure. The residue was purified by a silica gel column chromatography to obtain 0.8 g of the target compound. MS (ESI) m/z (M+H)⁺=315.0.

### Step 4: Preparation of (4-(benzo[d]oxazol-5-yl) imidazo[1,2-a]quinoxalin-7-yl)(piperidin-1-yl)methanone

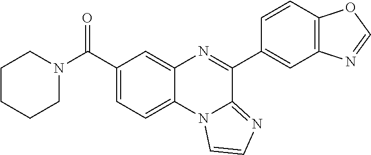 [SMILES: O=C(c1ccc2c(c1)nc(-c1ccc3ocnc3c1)c1nccn12)N1CCCCC1]

(4-chloroimidazo[1,2-a]quinoxalin-7-yl)(piperidin-1-yl)methanone (20 mg, 0.06 mmol), 5-(4,4,5,5-tetramethyl-1,3,2-dioxaborolan-2-yl)benzo[d]oxazole (30 mg, 0.12 mmol), [1,1′-bis(diphenylphosphino)ferrocene]dichloropalladium (5 mg, 0.006 mmol) and potassium carbonate (41 mg, 0.3 mmol) were dissolved in a mixed solution (5 mL) of dioxane and water, and reacted in an oil bath with stirring at 85° C. under nitrogen protection for 0.5 hours. After the disappearance of the raw materials as monitored by LCMS, the reaction was quenched by adding water. The residue was extracted with ethyl acetate for 3 times. The organic phases were combined, dried over anhydrous sodium sulfate, filtered, and concentrated under reduced pressure. The residue was purified by a reverse phase preparative chromatography to obtain 10 mg of the target compound. MS (ESI) m/z (M+H)⁺=398.1. ¹H NMR (400 MHz, DMSO-d₆) δ 9.34 (d, J=1.7 Hz, 1H), 8.95-8.90 (m, 1H), 8.86 (dd, J=8.7, 1.7 Hz, 1H), 8.82 (s, 1H), 8.44 (d, J=8.4 Hz, 1H), 8.05 (d, J=1.8 Hz, 1H), 7.96-7.91 (m, 2H), 7.70 (dd, J=8.4, 1.8 Hz, 1H), 3.59 (s, 4H), 1.58 (s, 6H).

### Example 3: Preparation of 2-methyl-5-(1-methyl-7-(piperidine-1-carbonyl) imidazo[1,2-a]quinoxalin-4-yl) isoindolin-1-one

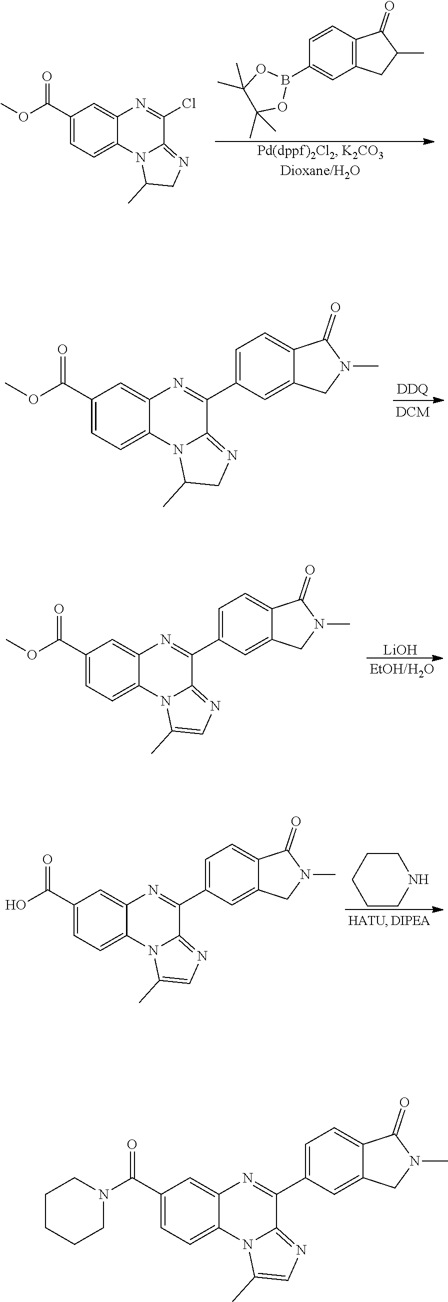

### Step 1: Preparation of methyl 1-methyl-4-(2-methyl-1-oxoisoindolin-5-yl)-1,2-dihydroimidazo[1,2-a]quinoxaline-7-carboxylate

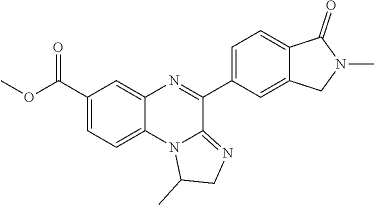

Methyl 1-methyl-4-chloro-1,2-dihydroimidazo[1,2-a]quinoxaline-7-carboxylate (103 mg), 4,4,5,5-tetramethyl-2-phenyl-1,3,2-dioxaborolane (155 mg), [1,1′-bis(diphenylphosphino)ferrocene]dichloropalladium (32 mg), and potassium carbonate (157 mg) were dissolved in a mixed solution (5 mL) of dioxane and water and reacted at 85° C. under nitrogen protection for 0.5 hours. After the disappearance of the raw materials as monitored by LCMS, the reaction was quenched by adding water and extracted with ethyl acetate for 3 times. The organic phases were combined, dried over anhydrous sodium sulfate, filtered, and concentrated under reduced pressure. The residue was purified by a silica gel column chromatography to obtain 70 mg of the target compound. MS (ESI) m/z (M+H)⁺=389.2.

### Step 2: Preparation of methyl 1-methyl-4-(2-methyl-1-oxoisoindolin-5-yl) imidazo[1,2-a]quinoxaline-7-carboxylate

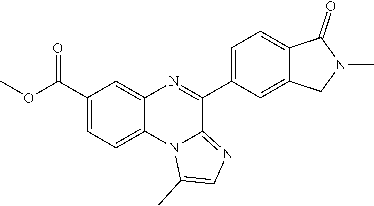

Methyl 1-methyl-4-(2-methyl-1-oxoisoindolin-5-yl)-1,2-dihydroimidazo[1,2-a]quinoxaline-7-carboxylate (180 mg) and 2,3-dichloro-5,6-dicyanobenzoquinone (313.3 mg) were dissolved in dichloromethane (15 mL) and reacted at room temperature for 2 hours. After the disappearance of the raw materials as monitored by LCMS, the reaction was quenched by adding water, and it was extracted with ethyl acetate for 3 times. The organic phases were combined, dried over anhydrous sodium sulfate, and concentrated under reduced pressure. The residue was purified by a silica gel column chromatography to obtain 150 mg of the target compound. MS (ESI) m/z (M+H)⁺=387.2.

### Step 3: Preparation of 1-methyl-4-(2-methyl-1-oxoisoindolin-5-yl) imidazo[1,2-a]quinoxaline-7-carboxylic acid

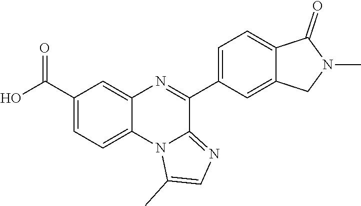 [SMILES: Cc1cnc2c(-c3ccc4c(c3)CN(C)C4=O)nc3cc(C(=O)O)ccc3n12]

Methyl 1-methyl-4-(2-methyl-1-oxoisoindolin-5-yl) imidazo[1,2-a]quinoxaline-7-carboxylate (150 mg) and lithium hydroxide (93.41 mg) were dissolved in a mixed solution (10 mL) of ethanol and water and reacted at 85° C. for 0.5 hours. After the disappearance of the raw materials as monitored by LCMS, the reaction was quenched by adding water, and it was extracted with ethyl acetate for 3 times. The organic phases were combined, dried over anhydrous sodium sulfate, and concentrated under reduced pressure. The residue was purified by a silica gel column chromatography to obtain 81 mg of the target compound. MS (ESI) m/z (M+H)⁺=375.1.

### Step 4: Preparation of 2-methyl-5-(1-methyl-7-(piperidine-1-carbonyl) imidazo[1,2-a]quinoxalin-4-yl) isoindolin-1-one

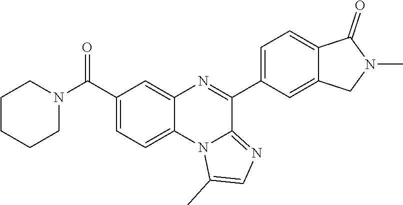 [SMILES: Cc1cnc2c(-c3ccc4c(c3)CN(C)C4=O)nc3cc(C(=O)N4CCCCC4)ccc3n12]

1-methyl-4-(2-methyl-1-oxoisoindolin-5-yl) imidazo[1,2-a]quinoxaline-7-carboxylic acid (20 mg), piperidine (13.54 mg), HATU (40.3 mg), and DIPEA (0.05 mL) were dissolved in DMF solution (10 mL) and reacted at room temperature for 1 hour. After the disappearance of the raw materials as monitored by LCMS, the reaction was quenched by adding water, and it was extracted with ethyl acetate for 3 times. The organic phases were combined, dried over anhydrous sodium sulfate, filtered, and concentrated under reduced pressure. The residue was purified by a silica gel column chromatography and then by a reverse phase preparative chromatography to obtain 5.25 mg of the target compound. MS (ESI) m/z (M+H)⁺=440.2. ¹H NMR (400 MHz, Chloroform-d) δ 8.74 (s, 2H), 8.43 (s, 1H), 8.26 (s, 1H), 8.02 (s, 1H), 7.70 (d, J=30.5 Hz, 2H), 4.54 (s, 2H), 3.79 (s, 2H), 3.48 (s, 2H), 3.26 (s, 3H), 3.06 (s, 3H), 1.82-1.63 (m, 6H).

### Example 4: Preparation of 2-methyl-5-(7′-(piperidine-1-carbonyl)-1′H-spiro[cyclopropane-1,2′-imidazo[1,2-a]quinoxaline]-4′-yl) isoindolin-1-one

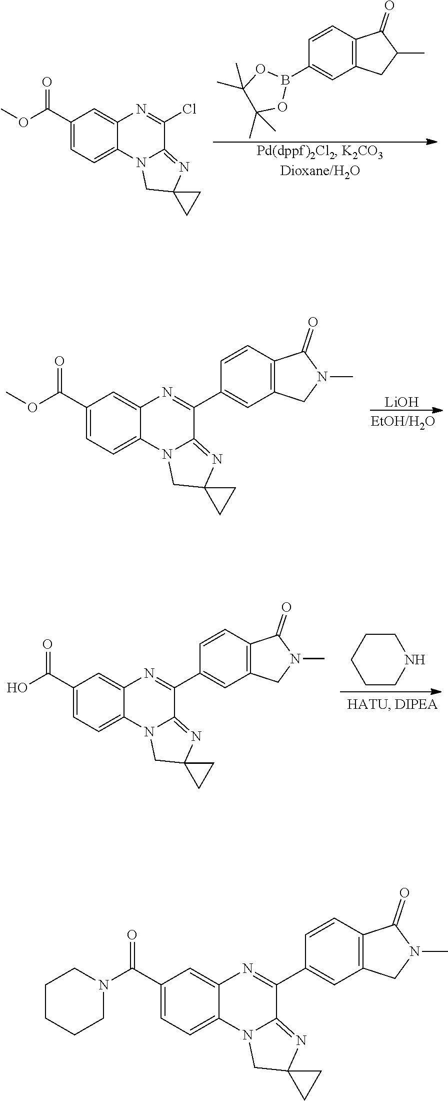

### Step 1: Preparation of methyl 4′-(2-methyl-1-oxoisoindolin-5-yl)-1′H-spiro[cyclopropane-1,2′-imidazo[1,2-a]quinoxaline]-7′-carboxylate

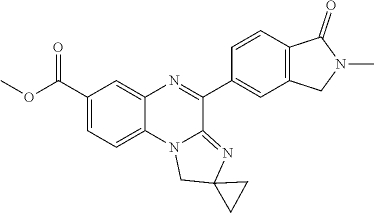

Methyl 4′-chloro-l′H-spiro[cyclopropane-1,2′-imidazo[1,2-a]quinoxaline]-7′-carboxylate (82 mg), 2-methyl-5-(4,4,5,5-tetramethyl-1,3,2-dioxaborolan-2-yl)-2,3-dihydro-1H-inden-1-one (152.4 mg), [1,1′-bis(diphenylphosphino)ferrocene]dichloropalladium (24.3 mg), and potassium carbonate (116 mg) were dissolved in a mixed solution (5 mL) of dioxane and water and reacted at 85° C. under nitrogen protection for 0.5 hours. After the disappearance of the raw materials as monitored by LCMS, the reaction was quenched by adding water and extracted with ethyl acetate for 3 times. The organic phases were combined, dried over anhydrous sodium sulfate, filtered, and concentrated under reduced pressure. The residue was purified by a silica gel column chromatography to obtain 78 mg of the target compound. MS (ESI) m/z (M+H)⁺=401.1.

### Step 2: Preparation of 4′-(2-methyl-1-oxoisoindolin-5-yl)-1′H-spiro[cyclopropane-1,2′-imidazo[1,2-a]quinoxaline]-7′-carboxylic acid

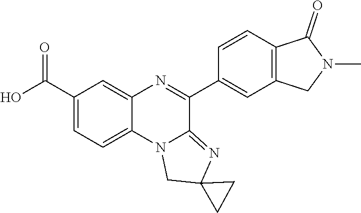 [SMILES: CN1Cc2cc(C3=Nc4cc(C(=O)O)ccc4N4CC5(CC5)N=C34)ccc2C1=O]

Methyl 4′-(2-methyl-1-oxoisoindolin-5-yl)-1′H-spiro[cyclopropane-1,2′-imidazo[1,2-a]quinoxaline]-7′-carboxylate (78 mg) and lithium hydroxide (45.5 mg) were dissolved in a mixed solution (8 mL) of ethanol and water and reacted at 85° C. for 0.5 hours. After the disappearance of the raw materials as monitored by LCMS, the reaction system was concentrated under reduced pressure to obtain 75 mg of the crude target compound. MS (ESI) m/z (M+H)⁺=387.1.

### Step 3: Preparation of 2-methyl-5-(7′-(piperidine-1-carbonyl)-1′H-spiro[cyclopropane-1,2′-imidazo[1,2-a]quinoxaline]-4′-yl) isoindolin-1-one

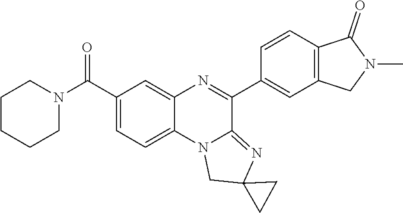 [SMILES: CN1Cc2cc(C3=Nc4cc(C(=O)N5CCCCC5)ccc4N4CC5(CC5)N=C34)ccc2C1=O]

4′-(2-methyl-1-oxoisoindolin-5-yl)-1′H-spiro[cyclopropane-1,2′-imidazo[1,2-a]quinoxaline]-7′-carboxylic acid (75 mg), piperidine (32.36 mg), HATU (144.5 mg), and DIPEA (0.32 mL) were dissolved in DMF solution (8 mL) and reacted at room temperature for 1 hour. After the disappearance of the raw materials as monitored by LCMS, the reaction was quenched by adding water, and it was extracted with ethyl acetate for 3 times. The organic phases were combined, dried over anhydrous sodium sulfate, filtered, and concentrated under reduced pressure. The residue was purified by a silica gel column chromatography to obtain 26 mg of the target compound. MS (ESI) m/z (M+H)⁺=454.2. ¹H NMR (400 MHz, DMSO-d₆) δ 8.60 (d, J=7.5 Hz, 2H), 7.77-7.73 (m, 1H), 7.61 (d, J=1.8 Hz, 1H), 7.46 (dd, J=8.2, 1.9 Hz, 1H), 6.88 (d, J=8.3 Hz, 1H), 4.53 (s, 2H), 4.16 (s, 2H), 3.49 (s, 4H), 3.10 (s, 3H), 1.63 (t, J=5.8 Hz, 2H), 1.54 (s, 4H), 1.19 (q, J=4.6 Hz, 2H), 0.98-0.90 (m, 2H).

### Example 5: Preparation of 2-methyl-5-(7-(piperidine-1-carbonyl)-[1,2,4]triazolo[4,3-a]quinoxalin-4-yl) isoindolin-1-one

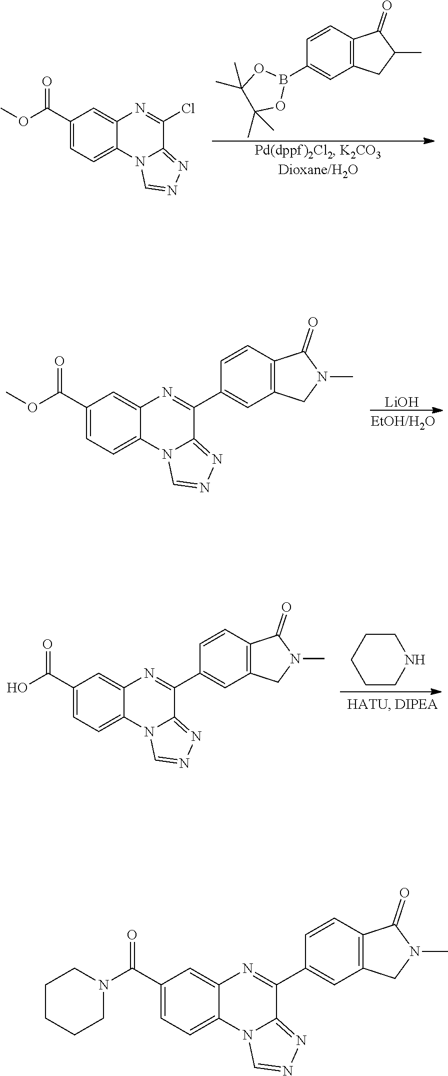

### Step 1: Preparation of methyl 4-(2-methyl-1-oxoisoindolin-5-yl)-[1,2,4]triazolo[4,3-a]quinoxaline-7-carboxylate

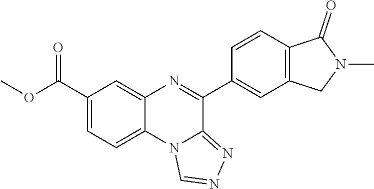

Methyl 4-chloro-[1,2,4]triazolo[4,3-a]quinoxaline-7-carboxylate (175 mg), 2-methyl-5-(4,4,5,5-tetramethyl-1,3,2-dioxaborolan-2-yl)-2,3-dihydro-1H-inden-1-one (366 mg), [1,1′-bis(diphenylphosphino)ferrocene]dichloropalladium dichloromethane complex (50 mg), and potassium carbonate (235 mg) were dissolved in a mixed solution (10 mL) of dioxane and water and reacted at 85° C. under nitrogen protection for 0.5 hours. After the disappearance of the raw materials as monitored by LCMS, the reaction was quenched by adding water, and it was extracted with ethyl acetate for 3 times. The organic phases were combined, dried over anhydrous sodium sulfate, filtered, and concentrated under reduced pressure. The residue was purified by a silica gel column chromatography to obtain 145 mg of the target compound. MS (ESI) m/z (M+H)⁺=373.1.

### Step 2: Preparation of 4-(2-methyl-1-oxoisoindolin-5-yl)-[1,2,4]triazolo[4,3-a]quinoxaline-7-carboxylic acid

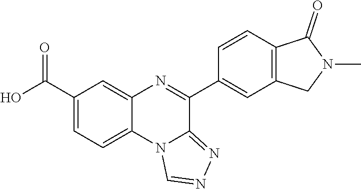 [SMILES: CN1Cc2cc(-c3nc4cc(C(=O)O)ccc4n4cnnc34)ccc2C1=O]

Methyl 4-(2-methyl-1-oxo-isoindolin-5-yl)-[1,2,4]triazolo[4,3-a]quinoxaline-7-carboxylate (145 mg) and lithium hydroxide (81.9 mg) were dissolved in a mixed solution (15 mL) of ethanol and water, and reacted at 85° C. for 0.5 hours. After the disappearance of the raw materials as monitored by LCMS, the reaction was quenched by adding water, and extracted with ethyl acetate for 3 times. The organic phases were combined, dried over anhydrous sodium sulfate, filtered, and concentrated under reduced pressure. The residue was purified by a silica gel column chromatography to obtain 87 mg of the target compound. MS (ESI) m/z (M+H)⁺=360.1.

### Step 3: Preparation of 2-methyl-5-(7-(piperidine-1-carbonyl)-[1,2,4]triazolo[4,3-a]quinoxalin-4-yl) isoindolin-1-one

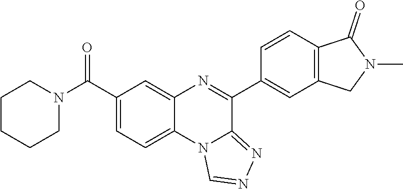 [SMILES: CN1Cc2cc(-c3nc4cc(C(=O)N5CCCCC5)ccc4n4cnnc34)ccc2C1=O]

4-(2-methyl-1-oxoisoindolin-5-yl)-[1,2,4]triazolo[4,3-a]quinoxaline-7-carboxylic acid (80 mg), piperidine (56.2 mg), HATU (167.3 mg), and DIPEA (0.2 mL) were dissolved in DMF solution (10 mL) and reacted at room temperature for 1 hour. After the disappearance of the raw materials as monitored by LCMS, the reaction was quenched by adding water, and extracted with ethyl acetate for 3 times. The organic phases were combined, dried over anhydrous sodium sulfate, filtered, and concentrated under reduced pressure. The residue was purified by a silica gel column chromatography to obtain 17 mg of the target compound. MS (ESI) m/z (M+H)⁺=427.1. ¹H NMR (400 MHz, DMSO-d₆) δ 10.21 (s, 1H), 9.00 (s, 1H), 8.90 (d, J=8.1 Hz, 1H), 8.49 (d, J=8.4 Hz, 1H), 8.08 (d, J=1.7 Hz, 1H), 7.86 (d, J=8.1 Hz, 1H), 7.79 (dd, J=8.4, 1.8 Hz, 1H), 4.59 (s, 2H), 3.59 (s, 4H), 3.08 (s, 3H), 1.55 (d, J=23.3 Hz, 6H).

### Examples 6-47

A series of compounds were prepared from corresponding commercial reagents and the products in the above Preparation Examples and Examples as raw materials, by using preparation methods similar to those of the above Examples, and structures and characterization data of the compounds are shown in Table 1.

TABLE 1

| | | Synthetic | |
|---|---|---|---|
| | | method/ | |
| | | reference | |
| | | Example/ | |
| Ex- | | raw | |
| ample | Structure | materials | MS(M + H)⁺ and ¹H NMR |
| 6 | 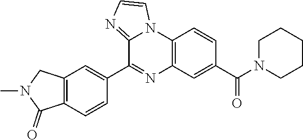 [SMILES: CN1Cc2cc(-c3nc4cc(C(=O)N5CCCCC5)ccc4n4ccnc34)ccc2C1=O] | Method I, Example 1 or 2, Preparation Example 1 | MS (ESI) m/z (M + H)⁺ = 426.1, ¹H NMR (400 MHz, DMSO-d₆) δ 9.01 (d, J = 12.6 Hz, 2H), 8.94 (d, J = 8.1 Hz, 1H), 8.52 (d, J = 8.4 Hz, 1H), 8.12 (s, 1H), 7.99 (s, 1H), 7.89 (d, J = 8.1 Hz, 1H), 7.83-7.75 (m, 1H), 4.64 (s, 2H), 3.66 (s, 4H), 3.14 (s, 3H), 1.60 (d, J = 40.7 Hz, 6H) |
| 7 | 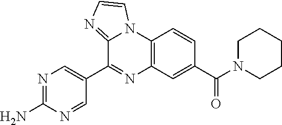 [SMILES: Nc1ncc(-c2nc3cc(C(=O)N4CCCCC4)ccc3n3ccnc23)cn1] | Method I, Example 1 or 2, Preparation Example 1 | MS (ESI) m/z (M + H)⁺ = 374.2, ¹H NMR (400 MHz, DMSO-d₆) δ 9.60 (s, 2H), 8.91-8.82 (m, 1H), 8.38 (d, J = 8.5 Hz, 1H), 7.95 (d, J = 1.8 Hz, 1H), 7.91-7.84 (m, 1H), 7.63 (dd, J = 8.3, 1.9 Hz, 1H), 7.30 (s, 2H), 3.58 (s, 4H), 1.57 (s, 4H), 1.47 (s, 2H) |
| 8 | 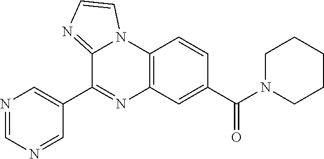 [SMILES: O=C(c1ccc2c(c1)nc(-c1cncnc1)c1nccn12)N1CCCCC1] | Method I, Example 1 or 2, Preparation Example 1 | MS (ESI) m/z (M + H)⁺ = 359.1, ¹H NMR (400 MHz, DMSO-d₆) δ 10.01 (s, 2H), 9.40 (s, 1H), 9.03 (s, 1H), 8.54 (d, J = 8.5 Hz, 1H), 8.15 (s, 1H), 8.02 (s, 1H), 7.86-7.78 (m, 1H), 3.66 (s, 4H), 1.65 (s, 4H), 1.54 (s, 2H) |
| 9 | 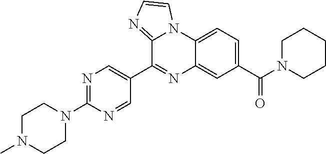 [SMILES: CN1CCN(c2ncc(-c3nc4cc(C(=O)N5CCCCC5)ccc4n4ccnc34)cn2)CC1] | Method I, Example 1 or 2, Preparation Example 1 | MS (ESI) m/z (M + H)⁺ = 457.2, ¹H NMR (400 MHz, DMSO-d₆) δ 9.67 (s, 2H), 8.86 (d, J = 1.3 Hz, 1H), 8.38 (d, J = 8.5 Hz, 1H), 7.95 (d, J = 1.8 Hz, 1H), 7.87 (d, J = 1.2 Hz, 1H), 7.63 (dd, J = 8.4, 1.8 Hz, 1H), 3.83 (t, J = 4.9 Hz, 4H), 3.58 (s, 4H), 2.35 (t, J = 5.0 Hz, 4H), 2.17 (s, 3H), 1.61-1.45 (m, 6H) |
| 10 | 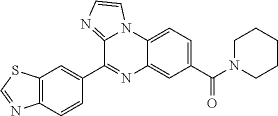 [SMILES: O=C(c1ccc2c(c1)nc(-c1ccc3ncsc3c1)c1nccn12)N1CCCCC1] | Method I, Example 1 or 2, Preparation Example 1 | MS (ESI) m/z (M + H)⁺ = 414.1, ¹H NMR (400 MHz, DMSO-d₆) δ 9.67 (d, J = 1.7 Hz, 1H), 9.51 (s, 1H), 8.92 (dd, J = 7.7, 1.5 Hz, 2H), 8.45 (d, J = 8.5 Hz, 1H), 8.24 (d, J = 8.7 Hz, 1H), 8.06 (d, J = 1.8 Hz, 1H), 7.94 (d, J = 1.2 Hz, 1H), 7.71 (dd, J = 8.5, 1.8 Hz, 1H), 3.59 (s, 4H), 1.54 (d, J = 39.0 Hz, 6H) |
| 11 | 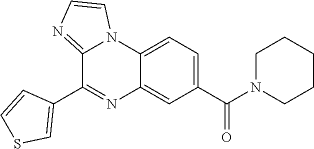 [SMILES: O=C(c1ccc2c(c1)nc(-c1ccsc1)c1nccn12)N1CCCCC1] | Method I, Example 1 or 2, Preparation Example 1 | MS (ESI) m/z (M + H)⁺ = 363.1, ¹H NMR (400 MHz, DMSO-d₆) δ 9.22 (dd, J = 3.0, 1.2 Hz, 1H), 8.85 (d, J = 1.2 Hz, 1H), 8.39 (d, J = 8.5 Hz, 1H), 8.19 (dd, J = 5.1, 1.1 Hz, 1H), 7.96 (d, J = 1.7 Hz, 1H), 7.89 (d, J = 1.1 Hz, 1H), 7.70-7.62 (m, 2H), 3.56 (s, 4H), 1.52 (d, J = 43.7 Hz, 6H) |
| 12 | 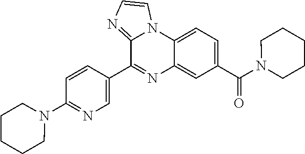 [SMILES: O=C(c1ccc2c(c1)nc(-c1ccc(N3CCCCC3)nc1)c1nccn12)N1CCCCC1] | Method I, Example 1 or 2, Preparation Example 1 | MS (ESI) m/z (M + H)⁺ = 441.2, ¹H NMR (400 MHz, DMSO-d₆) δ 9.65 (d, J = 2.5 Hz, 1H), 8.83 (q, J = 4.3, 3.4 Hz, 2H), 8.36 (d, J = 8.4 Hz, 1H), 7.98-7.89 (m, 1H), 7.85 (s, 1H), 7.64-7.56 (m, 1H), 6.94 (d, J = 9.2 Hz, 1H), 3.64 (t, J = 5.3 Hz, 8H), 1.69-1.57 (m, 4H), 1.55-1.41 (m, 8H) |
| 13 | 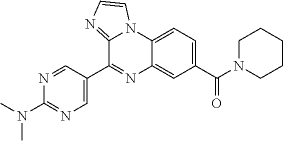 [SMILES: CN(C)c1ncc(-c2nc3cc(C(=O)N4CCCCC4)ccc3n3ccnc23)cn1] | Method I, Example 1 or 2, Preparation Example 1 | MS (ESI) m/z (M + H)⁺ = 402.2, ¹H NMR (400 MHz, DMSO-d6) δ 9.67 (s, 2H), 8.84 (d, J = 1.3 Hz, 1H), 8.37 (d, J = 8.5 Hz, 1H), 7.94 (d, J = 1.7 Hz, 1H), 7.87 (d, J = 1.2 Hz, 1H), 7.62 (dd, J = 8.4, 1.8 Hz, 1H), 3.56 (s, 4H), 3.19 (s, 6H), 1.52 (d, J = 46.3 Hz, 6H) |
| 14 | 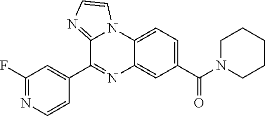 [SMILES: O=C(c1ccc2c(c1)nc(-c1ccnc(F)c1)c1nccn12)N1CCCCC1] | Method I, Example 1 or 2, Preparation Example 1 | MS (ESI) m/z (M + H)⁺ = 376.1, ¹H NMR (400 MHz, DMSO-d₆) δ 8.96 (d, J = 1.2 Hz, 1H), 8.62 (dt, J = 5.3, 1.7 Hz, 1H), 8.50-8.42 (m, 3H), 8.09 (d, J = 1.7 Hz, 1H), 7.95 (d, J = 1.2 Hz, 1H), 7.78 (dd, J = 8.5, 1.8 Hz, 1H), 3.59 (s, 2H), 3.30 (s, 2H), 1.65-1.42 (m, 6H) |
| 15 | 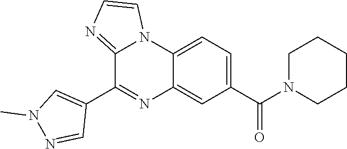 [SMILES: Cn1cc(-c2nc3cc(C(=O)N4CCCCC4)ccc3n3ccnc23)cn1] | Method I, Example 1 or 2, Preparation Example 1 | MS (ESI) m/z (M + H)⁺ = 361.2, ¹H NMR (400 MHz, DMSO-d₆) δ 8.90 (s, 1H), 8.82 (d, J = 1.3 Hz, 1H), 8.44 (s, 1H), 8.36 (d, J = 8.4 Hz, 1H), 7.87 (d, J = 1.8 Hz, 1H), 7.84 (d, J = 1.2 Hz, 1H), 7.60 (dd, J = 8.4, 1.8 Hz, 1H), 3.94 (s, 3H), 3.58 (s, 4H), 1.52 (d, J = 42.9 Hz, 6H) |
| 16 | 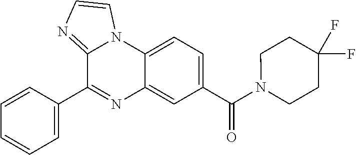 [SMILES: O=C(c1ccc2c(c1)nc(-c1ccccc1)c1nccn12)N1CCC(F)(F)CC1] | Method I, Example 1 or 2, Preparation Example 1 | MS (ESI) m/z (M + H)⁺ = 393.1, ¹H NMR (400 MHz, DMSO-d₆) δ 8.91 (s, 1H), 8.78-8.70 (m, 2H), 8.44 (d, J = 8.4 Hz, 1H), 8.14 (s, 1H), 7.90 (s, 1H), 7.77 (d, J = 8.5 Hz, 1H), 7.57-7.51 (m, 3H), 3.69 (s, 2H), 3.45 (s, 2H), 2.04 (s, 4H) |
| 17 | 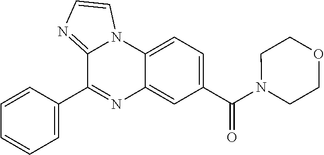 [SMILES: O=C(c1ccc2c(c1)nc(-c1ccccc1)c1nccn12)N1CCOCC1] | Method I, Example 1 or 2, Preparation Example 1 | MS (ESI) m/z (M + H)⁺ = 359.1, ¹H NMR (400 MHz, DMSO-d₆) δ 9.03 (d, J = 1.3 Hz, 1H), 8.87 (dt, J = 6.2, 1.9 Hz, 2H), 8.56 (d, J = 8.4 Hz, 1H), 8.20 (d, J = 1.7 Hz, 1H), 8.02 (d, J = 1.2 Hz, 1H), 7.86 (dd, J = 8.4, 1.8 Hz, 1H), 7.70-7.64 (m, 3H), 3.72 (s, 8H) |
| 18 | 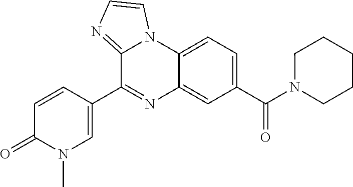 [SMILES: Cn1cc(-c2nc3cc(C(=O)N4CCCCC4)ccc3n3ccnc23)ccc1=O] | Method I, Example 1 or 2, Preparation Example 1 | MS (ESI) m/z (M + H)⁺ = 388.1, ¹H NMR (400 MHz, DMSO-d₆) δ 9.66 (d, J = 2.5 Hz, 1H), 8.87 (d, J = 1.3 Hz, 1H), 8.67 (dd, J = 9.6, 2.6 Hz, 1H), 8.39 (d, J = 8.4 Hz, 1H), 7.94 (d, J = 1.7 Hz, 1H), 7.88 (d, J = 1.2 Hz, 1H), 7.64 (dd, J = 8.5, 1.8 Hz, 1H), 6.57 (d, J = 9.6 Hz, 1H), 3.57 (s, 7H), 1.52 (d, J = 43.5 Hz, 6H) |
| 19 | 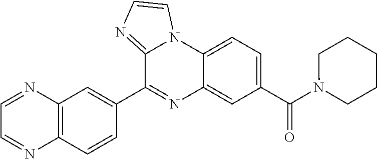 [SMILES: O=C(c1ccc2c(c1)nc(-c1ccc3nccnc3c1)c1nccn12)N1CCCCC1] | Method I, Example 1 or 2, Preparation Example 1 | MS (ESI) m/z (M + H)⁺ = 409.1, ¹H NMR (400 MHz, DMSO-d₆) δ 9.88 (d, J = 2.0 Hz, 1H), 9.16 (dd, J = 8.9, 2.0 Hz, 1H), 9.09-9.02 (m, 3H), 8.55 (d, J = 8.5 Hz, 1H), 8.31 (d, J = 8.9 Hz, 1H), 8.19 (d, J = 1.8 Hz, 1H), 8.08-8.03 (m, 1H), 7.82 (dd, J = 8.4, 1.8 Hz, 1H), 3.67 (s, 2H), 3.40 (s, 2H), 1.61 (d, J = 37.5 Hz, 6H) |
| 20 | 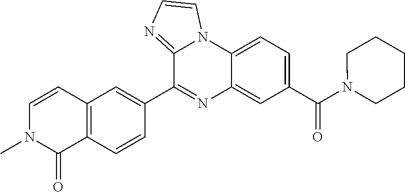 [SMILES: Cn1ccc2cc(-c3nc4cc(C(=O)N5CCCCC5)ccc4n4ccnc34)ccc2c1=O] | Method I, Example 1 or 2, Preparation Example 1 | MS (ESI) m/z (M + H)⁺ = 438.2, ¹H NMR (400 MHz, DMSO-d₆) δ 9.15 (d, J = 1.6 Hz, 1H), 9.00 (d, J = 1.3 Hz, 1H), 8.78 (dd, J = 8.5, 1.7 Hz, 1H), 8.52 (d, J = 8.5 Hz, 1H), 8.40 (d, J = 8.5 Hz, 1H), 8.13 (d, J = 1.7 Hz, 1H), 8.01 (d, J = 1.2 Hz, 1H), 7.80 (dd, J = 8.4, 1.8 Hz, 1H), 7.56 (d, J = 7.3 Hz, 1H), 6.81 (d, J = 7.3 Hz, 1H), 3.66 (s, 2H), 3.56 (s, 3H), 3.39 (s, 2H), 1.70-1.51 (m, 6H) |
| 21 | 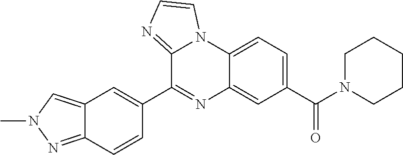 [SMILES: Cn1cc2cc(-c3nc4cc(C(=O)N5CCCCC5)ccc4n4ccnc34)ccc2n1] | Method I, Example 1 or 2, Preparation Example 1 | MS (ESI) m/z (M + H)⁺ = 411.2, ¹H NMR (400 MHz, DMSO-d₆) δ 9.68 (d, J = 1.6 Hz, 1H), 8.95 (d, J = 1.3 Hz, 1H), 8.63 (s, 1H), 8.60 (dd, J = 9.2, 1.7 Hz, 1H), 8.48 (d, J = 8.4 Hz, 1H), 8.07 (d, J = 1.8 Hz, 1H), 7.97 (d, J = 1.3 Hz, 1H), 7.78-7.71 (m, 2H), 4.23 (s, 3H), 3.66 (s, 4H), 1.71-1.52 (m, 6H) |
| 22 | 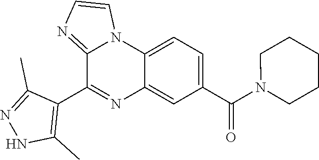 [SMILES: Cc1n[nH]c(C)c1-c1nc2cc(C(=O)N3CCCCC3)ccc2n2ccnc12] | Method I, Example 1 or 2, Preparation Example 1 | MS (ESI) m/z (M + H)⁺ = 375.2, ¹H NMR (400 MHz, DMSO-d₆) δ 12.58 (s, 1H), 8.86 (d, J = 1.3 Hz, 1H), 8.44 (d, J = 8.4 Hz, 1H), 7.96 (d, J = 1.7 Hz, 1H), 7.86 (d, J = 1.2 Hz, 1H), 7.69 (dd, J = 8.5, 1.8 Hz, 1H), 3.64 (s, 4H), 2.35 (d, J = 14.6 Hz, 6H), 1.58 (d, J = 48.1 Hz, 6H) |
| 23 | 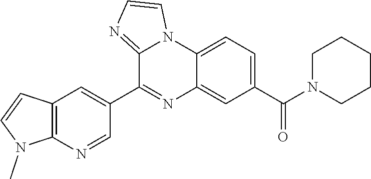 [SMILES: Cn1ccc2cc(-c3nc4cc(C(=O)N5CCCCC5)ccc4n4ccnc34)cnc21] | Method I, Example 1 or 2, Preparation Example 1 | MS (ESI) m/z (M + H)⁺ = 411.2, ¹H NMR (400 MHz, DMSO-d₆) δ 9.68 (d, J = 2.0 Hz, 1H), 9.50 (d, J = 2.0 Hz, 1H), 8.97 (d, J = 1.3 Hz, 1H), 8.49 (d, J = 8.5 Hz, 1H), 8.10 (d, J = 1.7 Hz, 1H), 7.99 (d, J = 1.2 Hz, 1H), 7.74 (dd, J = 8.5, 1.8 Hz, 1H), 7.64 (d, J = 3.4 Hz, 1H), 6.71 (d, J = 3.4 Hz, 1H), 3.92 (s, 3H), 3.66 (s, 4H), 1.65 (s, 6H) |
| 24 | 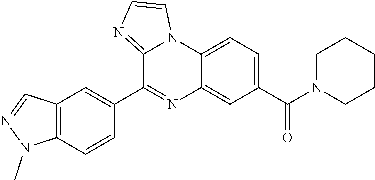 [SMILES: Cn1ncc2cc(-c3nc4cc(C(=O)N5CCCCC5)ccc4n4ccnc34)ccc21] | Method I, Example 1 or 2, Preparation Example 1 | MS (ESI) m/z (M + H)⁺ = 411.2, ¹H NMR (400 MHz, DMSO-d₆) δ 9.55 (d, J = 1.5 Hz, 1H), 8.89 (d, J = 1.3 Hz, 1H), 8.75 (dd, J = 9.0, 1.6 Hz, 1H), 8.41 (d, J = 8.4 Hz, 1H), 8.25 (s, 1H), 8.02 (d, J = 1.8 Hz, 1H), 7.91 (d, J = 1.2 Hz, 1H), 7.76 (d, J = 9.0 Hz, 1H), 7.66 (dd, J = 8.4, 1.8 Hz, 1H), 4.06 (s, 3H), 3.59 (s, 4H), 1.54 (d, J = 36.4 Hz, 6H) |
| 25 | 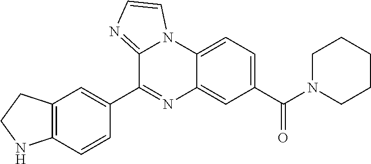 [SMILES: O=C(c1ccc2c(c1)nc(-c1ccc3c(c1)CCN3)c1nccn12)N1CCCCC1] | Method I, Example 1 or 2, Preparation Example 1 | MS (ESI) m/z (M + H)⁺ = 398.2, ¹H NMR (400 MHz, DMSO-d₆) δ 8.78 (d, J = 1.3 Hz, 1H), 8.69 (dd, J = 8.4, 1.8 Hz, 1H), 8.60 (s, 1H), 8.32 (d, J = 8.4 Hz, 1H), 7.87 (d, J = 1.7 Hz, 1H), 7.81 (d, J = 1.2 Hz, 1H), 7.54 (dd, J = 8.4, 1.7 Hz, 1H), 6.55 (d, J = 8.4 Hz, 1H), 6.22 (s, 1H), 3.51 (t, J = 8.6 Hz, 6H), 3.00 (t, J = 8.6 Hz, 2H), 1.53 (d, J = 35.8 Hz, 6H) |
| 26 | 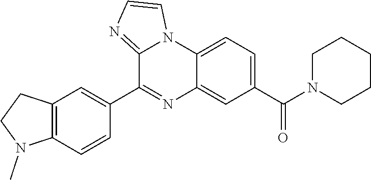 [SMILES: CN1CCc2cc(-c3nc4cc(C(=O)N5CCCCC5)ccc4n4ccnc34)ccc21] | Method I, Example 1 or 2, Preparation Example 1 | MS (ESI) m/z (M + H)⁺ = 412.2, ¹H NMR (400 MHz, DMSO-d₆) δ 8.86 (dt, J = 4.5, 2.5 Hz, 2H), 8.65 (s, 1H), 8.40 (d, J = 8.4 Hz, 1H), 7.95 (d, J = 1.7 Hz, 1H), 7.89 (d, J = 1.2 Hz, 1H), 7.65-7.59 (m, 1H), 6.64 (d, J = 8.5 Hz, 1H), 3.71-3.46 (m, 6H), 3.05 (t, J = 8.4 Hz, 2H), 2.85 (s, 3H), 1.60 (d, J = 35.4 Hz, 6H) |
| 27 | 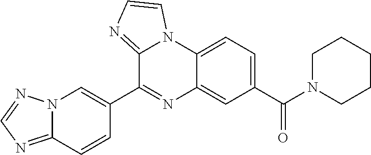 [SMILES: O=C(c1ccc2c(c1)nc(-c1ccc3ncnn3c1)c1nccn12)N1CCCCC1] | Method I, Example 1 or 2, Preparation Example 1 | MS (ESI) m/z (M + H)⁺ = 398.1, ¹H NMR (400 MHz, DMSO-d₆) δ 9.03 (s, 1H), 8.92 (d, J = 9.4 Hz, 1H), 8.68 (s, 1H), 8.54 (d, J = 8.5 Hz, 1H), 8.42 (s, 1H), 8.16 (s, 1H), 8.10- 8.04 (m, 2H), 7.81 (d, J = 8.4 Hz, 1H), 3.66 (s, 4H), 1.65 (s, 6H) |
| 28 | 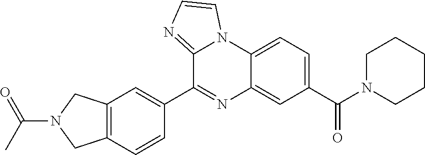 [SMILES: CC(=O)N1Cc2ccc(-c3nc4cc(C(=O)N5CCCCC5)ccc4n4ccnc34)cc2C1] | Method I, Example 1 or 2, Preparation Example 1 | MS (ESI) m/z (M + H)⁺ = 440.2, ¹H NMR (400 MHz, Chloroform-d) δ 8.81- 8.55 (m, 2H), 8.23 (d, J = 3.2 Hz, 2H), 7.99 (d, J = 8.3 Hz, 1H), 7.93 (s, 1H), 7.76 (d, J = 8.2 Hz, 1H), 7.55-7.44 (m, 1H), 4.94 (dd, J = 19.8, 11.5 Hz, 4H), 3.79 (s, 2H), 3.47 (s, 2H), 2.22 (s, 3H), 1.73 (s, 6H) |
| 29 | 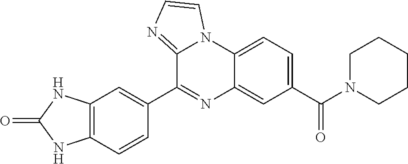 [SMILES: O=C(c1ccc2c(c1)nc(-c1ccc3[nH]c(=O)[nH]c3c1)c1nccn12)N1CCCCC1] | Method I, Example 1 or 2, Preparation Example 1 | MS (ESI) m/z (M + H)⁺ = 413.1, ¹H NMR (400 MHz, DMSO-d₆) δ 10.88 (s, 1H), 10.81 (s, 1H), 8.86 (d, J = 1.3 Hz, 1H), 8.62-8.54 (m, 2H), 8.39 (d, J = 8.6 Hz, 1H), 7.96 (d, J = 1.8 Hz, 1H), 7.88 (d, J = 1.2 Hz, 1H), 7.64 (dd, J = 8.4, 1.8 Hz, 1H), 7.07 (d, J = 8.9 Hz, 1H), 3.58 (s, 4H), 1.52 (d, J = 44.2 Hz, 6H) |
| 30 | 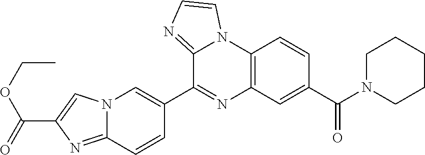 [SMILES: CCOC(=O)c1cn2cc(-c3nc4cc(C(=O)N5CCCCC5)ccc4n4ccnc34)ccc2n1] | Method I, Example 1 or 2, Preparation Example 1 | MS (ESI) m/z (M + H)⁺ = 469.2, ¹H NMR (400 MHz, DMSO-d₆) δ 10.45 (t, J = 1.3 Hz, 1H), 9.02 (d, J = 1.4 Hz, 1H), 8.92 (s, 1H), 8.60 (dd, J = 9.7, 1.8 Hz, 1H), 8.52 (d, J = 8.5 Hz, 1H), 8.11 (d, J = 1.8 Hz, 1H), 8.02 (d, J = 1.3 Hz, 1H), 7.84-7.77 (m, 2H), 4.35 (q, J = 7.1 Hz, 2H), 3.66 (s, 4H), 1.60 (d, J = 41.9 Hz, 6H), 1.35 (t, J = 7.1 Hz, 3H) |
| 31 | 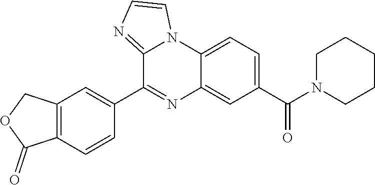 [SMILES: O=C1OCc2cc(-c3nc4cc(C(=O)N5CCCCC5)ccc4n4ccnc34)ccc21] | Method I, Example 1 or 2, Preparation Example 1 | MS (ESI) m/z (M + H)⁺ = 413.1, ¹H NMR (400 MHz, DMSO-d₆) δ 9.03 (s, 1H), 8.97-8.89 (m, 2H), 8.46 (d, J = 8.5 Hz, 1H), 8.06 (d, J = 1.8 Hz, 1H), 8.01 (d, J = 8.1 Hz, 1H), 7.94 (d, J = 1.2 Hz, 1H), 7.74 (dd, J = 8.4, 1.8 Hz, 1H), 5.52 (s, 2H), 3.59 (s, 4H), 1.53 (d, J = 39.1 Hz, 6H) |
| 32 | 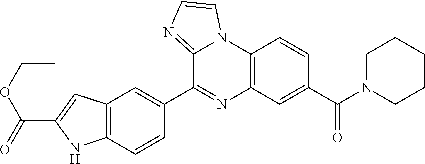 [SMILES: CCOC(=O)c1cc2cc(-c3nc4cc(C(=O)N5CCCCC5)ccc4n4ccnc34)ccc2[nH]1] | Method I, Example 1 or 2, Preparation Example 1 | MS (ESI) m/z (M + H)⁺ = 468.2, ¹H NMR (400 MHz, DMSO-d₆) δ 12.11 (s, 1H), 9.39 (d, J = 1.6 Hz, 1H), 8.88 (d, J = 1.3 Hz, 1H), 8.66 (dd, J = 8.9, 1.6 Hz, 1H), 8.41 (d, J = 8.4 Hz, 1H), 8.00 (d, J = 1.7 Hz, 1H), 7.91 (d, J = 1.2 Hz, 1H), 7.65 (dd, J = 8.4, 1.8 Hz, 1H), 7.57 (d, J = 8.9 Hz, 1H), 7.32 (s, 1H), 4.32 (q, J = 7.1 Hz, 2H), 3.59 (s, 4H), 1.54 (d, J = 34.7 Hz, 6H), 1.31 (t, J = 7.1 Hz, 3H) |
| 33 | 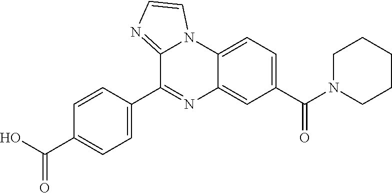 [SMILES: O=C(O)c1ccc(-c2nc3cc(C(=O)N4CCCCC4)ccc3n3ccnc23)cc1] | Method I, Example 1 or 2, Preparation Example 1 | MS (ESI) m/z (M + H)⁺ = 401.1, ¹H NMR (400 MHz, Chloroform-d) δ 8.82 (d, J = 8.2 Hz, 2H), 8.29 (d, J = 8.1 Hz, 2H), 8.23 (d, J = 3.4 Hz, 2H), 7.99 (d, J = 8.4 Hz, 1H), 7.94 (s, 1H), 7.78 (d, J = 8.4 Hz, 1H), 3.80 (s, 2H), 3.48 (s, 2H), 1.68 (d, J = 49.4 Hz, 6H) |
| 34 | 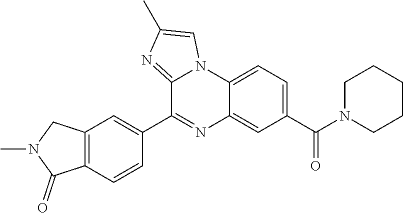 [SMILES: Cc1cn2c(n1)c(-c1ccc3c(c1)CN(C)C3=O)nc1cc(C(=O)N3CCCCC3)ccc12] | Method II, Example 3, Preparation Example 4 | MS (ESI) m/z (M + H)⁺ = 440.2, ¹H NMR (400 MHz, Chloroform-d) δ 8.84 (s, 1H), 8.78 (d, J = 7.4 Hz, 1H), 8.20 (s, 1H), 8.02 (d, J = 8.2 Hz, 2H), 7.94 (s, 1H), 7.76 (s, 1H), 4.55 (s, 2H), 3.78 (s, 2H), 3.47 (s, 2H), 3.26 (s, 3H), 2.66 (s, 3H), 1.67 (d, J = 47.5 Hz, 6H) |
| 35 | 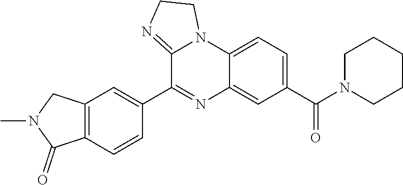 [SMILES: CN1Cc2cc(C3=Nc4cc(C(=O)N5CCCCC5)ccc4N4CCN=C34)ccc2C1=O] | Method I, Example 1 or 2, Preparation Example 5 | MS (ESI) m/z (M + H)⁺ = 428.2, ¹H NMR (400 MHz, Chloroform-d) δ 8.57 (s, 1H), 8.51 (d, J = 8.0 Hz, 1H), 7.92 (d, J = 7.9 Hz, 1H), 7.79 (d, J = 1.7 Hz, 1H), 7.53 (d, J = 7.8 Hz, 1H), 6.83 (d, J = 8.1 Hz, 1H), 4.46 (s, 2H), 4.29 (d, J = 9.6 Hz, 2H), 4.17 (d, J = 10.2 Hz, 2H), 3.49 (s, 4H), 3.22 (s, 3H), 1.67 (d, J = 28.7 Hz, 6H) |
| 36 | 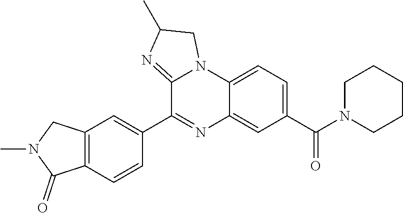 [SMILES: CC1CN2C(=N1)C(c1ccc3c(c1)CN(C)C3=O)=Nc1cc(C(=O)N3CCCCC3)ccc12] | Method I, Example 1 or 2, Preparation Example 4 | MS (ESI) m/z (M + H)⁺ = 442.2, ¹H NMR (400 MHz, Chloroform-d) δ 8.60 (s, 1H), 8.55 (d, J = 8.0 Hz, 1H), 7.92 (d, J = 8.0 Hz, 1H), 7.77 (d, J = 1.8 Hz, 1H), 7.51 (dd, J = 8.2, 1.8 Hz, 1H), 6.79 (d, J = 8.3 Hz, 1H), 4.58 (q, J = 8.9, 7.1 Hz, 1H), 4.47 (s, 2H), 4.20 (t, J = 10.7 Hz, 1H), 3.75-3.42 (m, 5H), 3.23 (s, 3H), 1.74-1.59 (m, 6H), 1.49 (d, J = 6.6 Hz, 3H) |
| 37 | 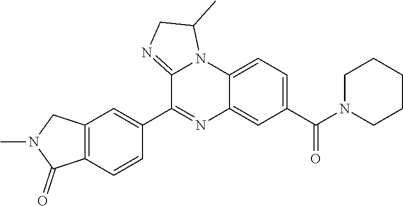 [SMILES: CC1CN=C2C(c3ccc4c(c3)CN(C)C4=O)=Nc3cc(C(=O)N4CCCCC4)ccc3N21] | Method I, Example 1 or 2, Preparation Example 3 | MS (ESI) m/z (M + H)⁺ = 442.2, ¹H NMR (400 MHz, DMSO-d₆) δ 8.77 (s, 1H), 8.66 (dd, J = 8.2, 1.5 Hz, 1H), 7.77 (d, J = 8.1 Hz, 1H), 7.64 (d, J = 1.9 Hz, 1H), 7.45 (dd, J = 8.3, 1.9 Hz, 1H), 7.17 (d, J = 8.4 Hz, 1H), 4.84-4.69 (m, 1H), 4.55 (s, 2H), 4.22 (dd, J = 15.7, 10.7 Hz, 1H), 3.74 (dd, J = 15.8, 4.3 Hz, 1H), 3.49 (s, 4H), 3.11 (s, 3H), 1.63 (q, J = 5.6 Hz, 2H), 1.54 (s, 4H), 1.35 (d, J = 6.2 Hz, 3H) |
| 38 | 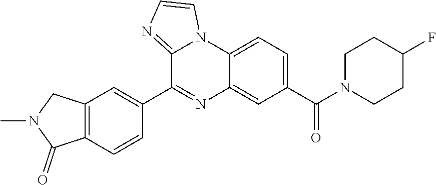 [SMILES: CN1Cc2cc(-c3nc4cc(C(=O)N5CCC(F)CC5)ccc4n4ccnc34)ccc2C1=O] | Method I, Example 1 or 2, Preparation Example 1 | MS (ESI) m/z (M + H)⁺ = 444.1, ¹H NMR (400 MHz, DMSO-d₆) δ 8.99-8.91 (m, 2H), 8.90-8.85 (m, 1H), 8.46 (d, J = 8.5 Hz, 1H), 8.11 (d, J = 1.8 Hz, 1H), 7.96-7.89 (m, 1H), 7.82 (d, J = 8.0 Hz, 1H), 7.76 (dd, J = 8.5, 1.8 Hz, 1H), 4.89 (d, J = 48.2 Hz, 1H), 4.57 (s, 2H), 3.57 (d, J = 74.6 Hz, 4H), 3.07 (s, 3H), 2.00-1.84 (m, 2H), 1.73 (s, 2H) |
| 39 | 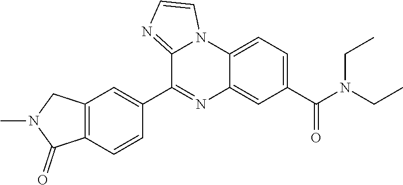 | Method I, Example 1 or 2, Preparation Example 1 | MS (ESI) m/z (M + H)⁺ = 417.1, ¹H NMR (400 MHz, DMSO-d₆) δ 8.99-8.90 (m, 2H), 8.91-8.84 (m, 1H), 8.45 (d, J = 8.5 Hz, 1H), 8.02 (d, J = 1.8 Hz, 1H), 7.93 (d, J = 1.4 Hz, 1H), 7.82 (d, J = 8.1 Hz, 1H), 7.71 (dd, J = 8.5, 1.8 Hz, 1H), 4.57 (s, 2H), 3.44 (s, 4H), 3.07 (s, 3H), 1.18-1.02 (m, 6H) |
| 40 | 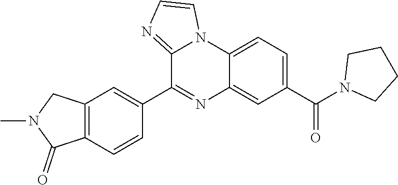 [SMILES: CN1Cc2cc(-c3nc4cc(C(=O)N5CCCC5)ccc4n4ccnc34)ccc2C1=O] | Method I, Example 1 or 2, Preparation Example 1 | MS (ESI) m/z (M + H)⁺ = 412.1, ¹H NMR (400 MHz, DMSO-d₆) δ 8.98-8.91 (m, 2H), 8.90-8.85 (m, 1H), 8.44 (d, J = 8.6 Hz, 1H), 8.21 (d, J = 1.8 Hz, 1H), 7.93 (d, J = 1.3 Hz, 1H), 7.89-7.79 (m, 2H), 4.57 (s, 2H), 3.48 (d, J = 7.2 Hz, 4H), 3.07 (s, 3H), 1.83 (dq, J = 19.5, 6.8 Hz, 4H) |
| 41 | 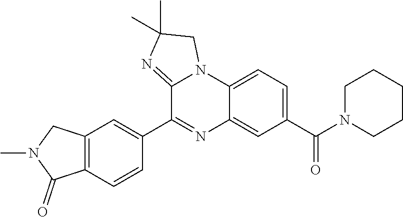 [SMILES: CN1Cc2cc(C3=Nc4cc(C(=O)N5CCCCC5)ccc4N4CC(C)(C)N=C34)ccc2C1=O] | Method I, Example 1 or 2, Preparation Example 6 | MS (ESI) m/z (M + H)⁺ = 456.2, ¹H NMR (400 MHz, DMSO-d₆) δ 8.69 (d, J = 8.6 Hz, 2H), 7.79 (d, J = 8.1 Hz, 1H), 7.64 (d, J = 1.8 Hz, 1H), 7.47 (dd, J = 8.3, 1.9 Hz, 1H), 6.99 (d, J = 8.3 Hz, 1H), 4.57 (s, 2H), 3.83 (s, 2H), 3.49 (s, 4H), 3.12 (s, 3H), 1.63 (s, 2H), 1.54 (s, 4H), 1.38 (s, 6H) |
| 42 | 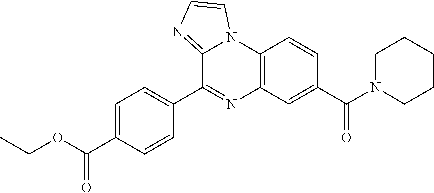 [SMILES: CCOC(=O)c1ccc(-c2nc3cc(C(=O)N4CCCCC4)ccc3n3ccnc23)cc1] | Method I, Example 1 or 2, Preparation Example 1 | MS (ESI) m/z (M + H)⁺ = 429.1, ¹H NMR (400 MHz, DMSO-d₆) δ 8.94-8.85 (m, 3H), 8.45 (d, J = 8.5 Hz, 1H), 8.11 (d, J = 8.5 Hz, 2H), 8.05 (d, J = 1.8 Hz, 1H), 7.93 (d, J = 1.2 Hz, 1H), 7.73 (dd, J = 8.4, 1.8 Hz, 1H), 4.32 (q, J = 7.1 Hz, 2H), 3.68-3.43 (m, 4H), 1.53 (d, J = 42.8 Hz, 6H), 1.31 (t, J = 7.1 Hz, 3H) |
| 43 | 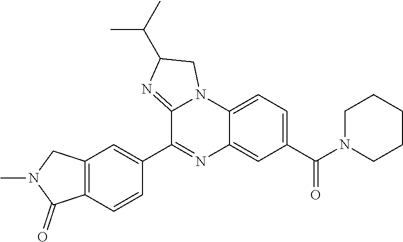 [SMILES: CC(C)C1CN2C(=N1)C(c1ccc3c(c1)CN(C)C3=O)=Nc1cc(C(=O)N3CCCCC3)ccc12] | Method I, Example 1 or 2, Preparation Example 8 | MS (ESI) m/z (M + H)⁺ = 470.2, ¹H NMR (400 MHz, DMSO-d₆) δ 8.75 (s, 1H), 8.71 (d, J = 8.1 Hz, 1H), 7.77 (d, J = 8.1 Hz, 1H), 7.63 (d, J = 1.8 Hz, 1H), 7.47 (dd, J = 8.3, 1.9 Hz, 1H), 7.01 (d, J = 8.3 Hz, 1H), 4.55 (s, 2H), 4.24-4.15 (m, 1H), 4.10 (t, J = 11.1 Hz, 1H), 3.80 (dd, J = 10.8, 8.6 Hz, 1H), 3.49 (s, 4H), 3.10 (s, 3H), 1.87 (q, J = 6.6 Hz, 1H), 1.63 (q, J = 5.6 Hz, 2H), 1.53 (s, 4H), 1.02 (d, J = 6.7 Hz, 3H), 0.93 (d, J = 6.7 Hz, 3H) |
| 44 | 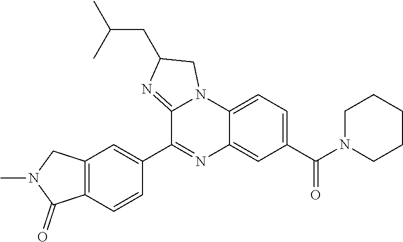 [SMILES: CC(C)CC1CN2C(=N1)C(c1ccc3c(c1)CN(C)C3=O)=Nc1cc(C(=O)N3CCCCC3)ccc12] | Method I, Example 1 or 2, Preparation Example 9 | MS (ESI) m/z (M + H)⁺ = 484.2, ¹H NMR (400 MHz, DMSO-d₆) δ 8.75 (s, 1H), 8.70 (dd, J = 8.1, 1.4 Hz, 1H), 7.77 (d, J = 8.0 Hz, 1H), 7.63 (d, J = 1.8 Hz, 1H), 7.46 (dd, J = 8.3, 1.8 Hz, 1H), 6.99 (d, J = 8.3 Hz, 1H), 4.55 (s, 2H), 4.41 (ddd, J = 15.5, 9.3, 5.5 Hz, 1H), 4.22 (t, J = 10.9 Hz, 1H), 3.67 (dd, J = 10.7, 8.7 Hz, 1H), 3.49 (s, 4H), 3.11 (s, 3H), 1.88 (dt, J = 13.4, 6.7 Hz, 1H), 1.70-1.61 (m, 3H), 1.57-1.42 (m, 5H), 0.98 (dd, J = 6.6, 3.3 Hz, 6H) |
| 45 | 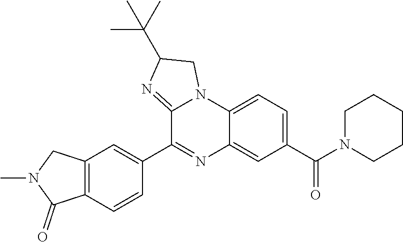 [SMILES: CN1Cc2cc(C3=Nc4cc(C(=O)N5CCCCC5)ccc4N4CC(C(C)(C)C)N=C34)ccc2C1=O] | Method I, Example 1 or 2, Preparation Example 10 | MS (ESI) m/z (M + H)⁺ = 484.2, ¹H NMR (400 MHz, DMSO-d₆) δ 8.77 (s, 1H), 8.72 (dd, J = 8.1, 1.5 Hz, 1H), 7.77 (d, J = 8.0 Hz, 1H), 7.63 (d, J = 1.8 Hz, 1H), 7.47 (dd, J = 8.3, 1.8 Hz, 1H), 7.04 (d, J = 8.3 Hz, 1H), 4.56 (s, 2H), 4.15 (dd, J = 11.6, 9.0 Hz, 1H), 4.04 (t, J = 11.4 Hz, 1H), 3.90 (dd, J = 11.1, 9.1 Hz, 1H), 3.49 (s, 4H), 3.11 (s, 3H), 1.67-1.61 (m, 2H), 1.53 (s, 4H), 0.97 (s, 9H) |

### Example 46: Preparation of 2-methyl-5-(8-(piperidine-1-carbonyl) imidazo[1,2-c]quinazolin-5-yl) isoindolin-1-one

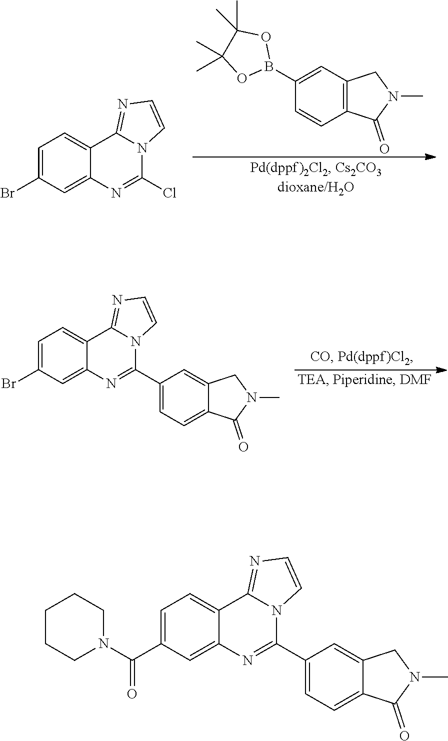

### Step 1: Preparation of 5-(8-bromoimidazo[1,2-c]quinazolin-5-yl)-2-methylisoindolin-1-one

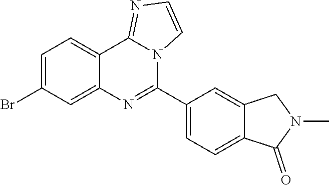 [SMILES: CN1Cc2cc(-c3nc4cc(Br)ccc4c4nccn34)ccc2C1=O]

8-bromo-5-chloroimidazo[1,2-c]quinazoline (300 mg), 2-methyl-5-(4,4,5,5-tetramethyl-1,3,2-dioxaborolan-2-yl) isoindolin-1-one (350 mg), cesium carbonate (452 mg), and [1,1′-bis(diphenylphosphino)ferrocene]dichloropalladium (39 mg) were weighed, into which 1,4-dioxane (10 mL) and pure water (2 mL) were added to dissolve. After argon replacement for three times, a reaction was carried out at 80° C. for 30 min. The reaction was completed as monitored by TLC and LCMS, and it was concentrated under reduced pressure, and purified by a silica gel column chromatography to obtain the title compound (320 mg). MS (ESI) m/z (M+H)⁺=393.0.

### Step 2: Preparation of 2-methyl-5-(8-(piperidine-1-carbonyl) imidazo[1,2-c]quinazolin-5-yl) isoindolin-1-one

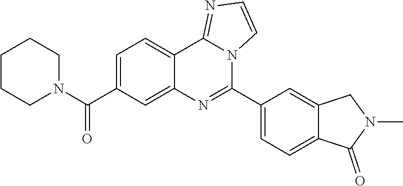 [SMILES: CN1Cc2cc(-c3nc4cc(C(=O)N5CCCCC5)ccc4c4nccn34)ccc2C1=O]

5-(8-bromoimidazo[1,2-c]quinazolin-5-yl)-2-methylisoindolin-1-one (320 mg) was weighed and dissolved in N,N-dimethylformamide (10 mL), into which triethylamine (0.22 mL), hexahydropyridine (0.22 mL), and [1,1′-bis(diphenylphosphino)ferrocene]dichloropalladium (60 mg) were added. After carbon monoxide replacement for three times, a reaction was carried out at 110° C. for 2 h. The reaction was completed as monitored by LCMS, and quenched by adding water, it was extracted with ethyl acetate for twice, washed with saturated brine, concentrated under reduced pressure, and separated by reverse phase preparative column to obtain 92.12 mg of the title compound. MS (ESI) m/z (M+H)⁺=426.2. ¹H NMR (400 MHz, DMSO-d₆) δ 8.51 (d, J=8.1 Hz, 1H), 8.18 (s, 1H), 8.06 (dd, J=7.9, 1.4 Hz, 1H), 7.99 (d, J=1.5 Hz, 1H), 7.94-7.89 (m, 2H), 7.71 (dd, J=7.9, 1.5 Hz, 2H), 4.62 (s, 2H), 3.65 (m, 2H), 3.33 (m, 2H), 3.15 (s, 3H), 1.57 (m, 6H).

### Example 47: Preparation of 2-methyl-5-(8-(piperidine-1-carbonyl) imidazo[1,2-c]quinazolin-5-yl) isoindolin-1-one

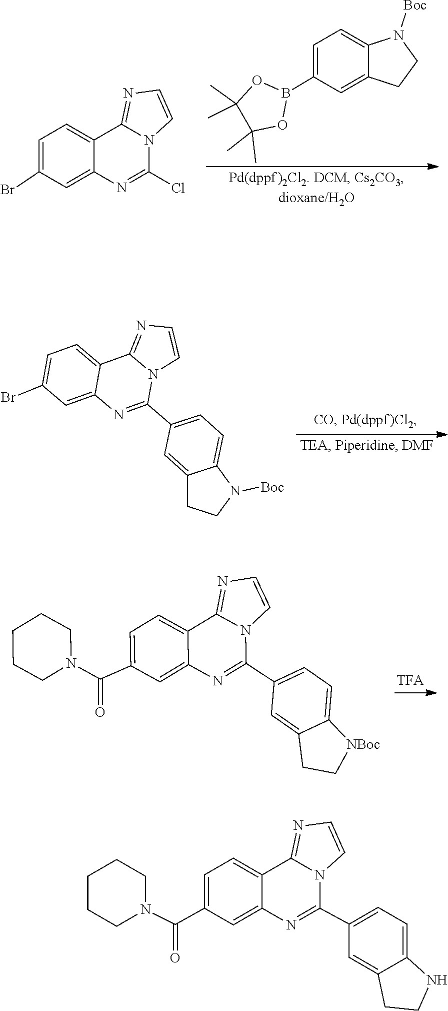

### Step 1: Preparation of tert-butyl 5-(8-bromoimidazo[1,2-c]quinazolin-5-yl) indole-1-carboxylate

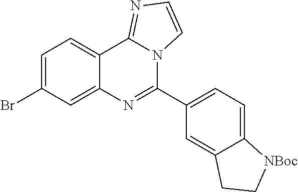 [SMILES: CC(C)(C)OC(=O)N1CCc2cc(-c3nc4cc(Br)ccc4c4nccn34)ccc21]

8-bromo-5-chloroimidazo[1,2-c]quinazoline (150 mg), tert-butyl 5-(tetramethyl-1,3,2-dioxaborolan-2-yl)-2,3-dihydro-1H-indole-1-carboxylate (220 mg), [1,1′-bis(diphenylphosphino)ferrocene]dichloropalladium (22 mg), and cesium carbonate (260 mg) were weighed and mixed in 1,4-dioxane (3 mL) and water (1 mL), and reacted at 80° C. under nitrogen protection for 2 h. The reaction was completed as monitored by LCMS. It was cooled to room temperature, quenched by adding water, and extracted with ethyl acetate for 3 times. The organic phases were combined, dried over anhydrous sodium sulfate, concentrated, and purified by silica gel column to obtain 140 mg of the title compound. MS (ESI) m/z (M+H)⁺=465.1.

### Step 2: Preparation of tert-butyl 5-(8-(piperidine-1-carbonyl) imidazo[1,2-c]quinazolin-5-yl) indole-1-carboxylate

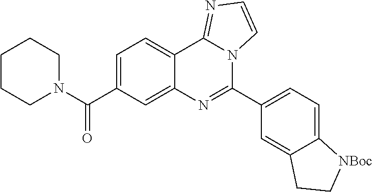 [SMILES: CC(C)(C)OC(=O)N1CCc2cc(-c3nc4cc(C(=O)N5CCCCC5)ccc4c4nccn34)ccc21]

Tert-butyl 5-(8-bromoimidazo[1,2-c]quinazolin-5-yl) indole-1-carboxylate (140 mg), triethylamine (92 mg), 4,5-bis(diphenylphosphino)-9,9-dimethylxanthene (18 mg), palladium acetate (4 mg), and 2,4,6-trichlorophenyl formate (102 mg) were weighed and mixed in dry 1,4-dioxane, and reacted under nitrogen protection at 120° C. for 6 h. Disappearance of raw materials was monitored by LCMS. After cooling to room temperature, piperidine (77 mg) was added thereto, and the reaction was carried out at room temperature for 2 h. The reaction was completed as monitored by LCMS. The solvent was evaporated under reduced pressure, filtered, and the filtrate was concentrated and then purified by a silica gel column to obtain 140 mg of the title compound. MS (ESI) m/z (M+H)⁺=498.1.

### Step 3: Preparation of (5-(indol-5-yl) imidazo[1,2-c]quinazolin-8-yl)(piperidin-1-yl)methanone

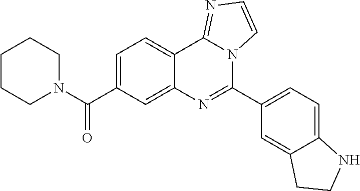 [SMILES: O=C(c1ccc2c(c1)nc(-c1ccc3c(c1)CCN3)n1ccnc21)N1CCCCC1]

Tert-butyl 5-(8-(piperidine-1-carbonyl) imidazo[1,2-c]quinazolin-5-yl) indole-1-carboxylate (140 mg) was weighed and dissolved in trifluoroacetic acid, and reacted at room temperature for 2 h. The reaction was completed as monitored by LCMS. The solvent was evaporated under reduced pressure, and the residue was adjusted to weak alkalinity with saturated sodium bicarbonate solution and concentrated to dryness. The residue was dissolved with ethyl acetate, filtered, and the filter cake was washed with ethyl acetate for several times. The filtrates were combined and concentrated to obtain a crude product, which was then separated by a reverse phase preparative column to obtain 13.13 mg of the title compound. MS (ESI) m/z (M+H)⁺=398.1. ¹H NMR (400 MHz, DMSO-d₆) δ 8.44-8.42 (d, J=8.1 Hz, 1H), 8.09-8.08 (m, 1H), 7.81 (m, 1H), 7.66 (s, 2H), 7.65-7.58 (m, 2H), 6.66-6.64 (d, J=8.1 Hz, 1H), 6.31 (s, 1H), 3.64-3.56 (m, 4H), 3.34 (m, 2H), 3.09-3.05 (t, J=8.6 Hz, 2H), 1.66-1.52 (m, 6H).

### Examples 48-82

A series of compounds were prepared from corresponding commercial reagents and the products in the above Preparation Examples and Examples as raw materials, by using preparation methods similar to those of the above Examples, and structures and characterization data of the compounds are shown in Table 2.

TABLE 2

| | | Synthetic | |
|---|---|---|---|
| | | method/ | |
| | | reference | |
| | | Example/ | |
| Ex- | | raw | |
| ample | Structure | materials | MS(M + H)⁺ and ¹H NMR |
| 48 | 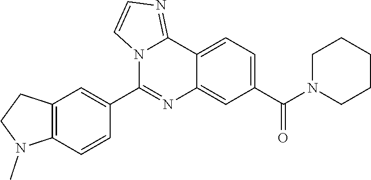 [SMILES: CN1CCc2cc(-c3nc4cc(C(=O)N5CCCCC5)ccc4c4nccn34)ccc21] | Method III, Example 46, Preparation Example 11 | MS (ESI) m/z (M + H)⁺ = 412.2, ¹H NMR (400 MHz, DMSO-d₆) δ 8.45-8.42 (d, J = 8.1 Hz, 1H), 8.08 (s, 1H), 7.82 (s, 1H), 7.73-7.71 (m, 1H), 7.66 (d, J = 3.0 Hz, 2H), 7.62-7.60 (m, 1H), 6.66-6.64 (d, J = 8.3 Hz, 1H), 3.64 (m, 2H), 3.48-3.44 (t, J = 8.4 Hz, 2H), 3.35 (m, 2H), 3.06-3.02 (t, J = 8.5 Hz, 2H), 2.84 (s, 3H), 1.67-1.42 (m, 6H) |
| 49 | 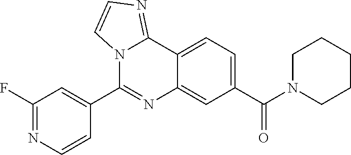 [SMILES: O=C(c1ccc2c(c1)nc(-c1ccnc(F)c1)n1ccnc21)N1CCCCC1] | Method III, Example 46, Preparation Example 11 | MS (ESI) m/z (M + H)⁺ = 376.1, ¹H NMR (400 MHz, DMSO-d₆) δ 8.56 (d, J = 5.1 Hz, 1H), 8.52 (d, J = 8.2 Hz, 1H), 8.07 (d, J = 1.5 Hz, 1H), 7.94 (d, J = 1.5 Hz, 1H), 7.92 (d, J = 5.1 Hz, 1H), 7.79-7.71 (m, 3H), 3.65 (m, 2H), 3.33(m, 2H), 1.57 (m, 6H) |
| 50 | 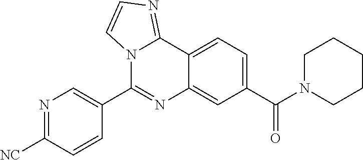 [SMILES: N#Cc1ccc(-c2nc3cc(C(=O)N4CCCCC4)ccc3c3nccn23)cn1] | Method III, Example 46, Preparation Example 11 | MS (ESI) m/z (M + H)⁺ = 383.1, ¹H NMR (400 MHz, Methanol-d₄) δ 9.33 (d, J = 2.2 Hz, 1H), 8.65 (dd, J = 8.0, 2.2 Hz, 1H), 8.61 (d, J = 8.2 Hz, 1H), 8.16 (d, J = 8.1 Hz, 1H), 8.05 (d, J = 1.9 Hz, 2H), 7.78 (dd, J = 8.2, 1.7 Hz, 1H), 7.72 (d, J = 1.7 Hz, 1H), 3.78 (m, 2H), 3.45 (m, 2H), 1.74 (m, 4H), 1.60 (m, 2H) |
| 51 | 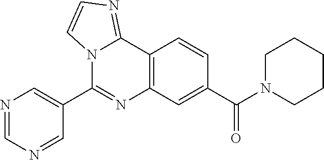 [SMILES: O=C(c1ccc2c(c1)nc(-c1cncnc1)n1ccnc21)N1CCCCC1] | Method III, Example 46, Preparation Example 11 | MS (ESI) m/z (M + H)⁺ = 359.1, ¹H NMR (400 MHz, Methanol-d₄) δ 9.43 (d, J = 2.8 Hz, 3H), 8.59 (d, J = 8.2 Hz, 1H), 8.10 (d, J = 1.7 Hz, 1H), 8.06-8.03 (m, 1H), 7.77 (dd, J = 8.2, 1.7 Hz, 1H), 7.72 (d, J = 1.7 Hz, 1H), 3.78 (m, 2H), 3.46 (m, 2H), 1.79-1.70 (m, 4H), 1.60 (m, 2H) |
| 52 | 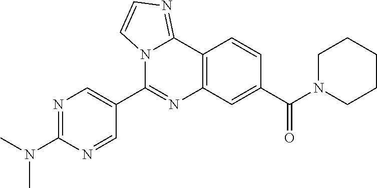 [SMILES: CN(C)c1ncc(-c2nc3cc(C(=O)N4CCCCC4)ccc3c3nccn23)cn1] | Method III, Example 46, Preparation Example 11 | MS (ESI) m/z (M + H)⁺ = 402.1, ¹H NMR (400 MHz, Methanol-d₄) δ 8.98 (s, 2H), 8.54 (d, J = 8.2 Hz, 1H), 8.11 (d, J = 1.8 Hz, 1H), 7.97 (d, J = 1.6 Hz, 1H), 7.69 (dt, J = 5.2, 2.6 Hz, 2H), 3.78 (m, 2H), 3.46 (m, 2H), 3.30 (s, 6H), 1.74 (m, 4H), 1.60 (m, 2H) |
| 53 | 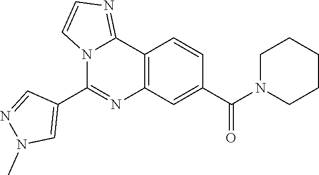 [SMILES: Cn1cc(-c2nc3cc(C(=O)N4CCCCC4)ccc3c3nccn23)cn1] | Method III, Example 46, Preparation Example 11 | MS (ESI) m/z (M + H)⁺ = 361.1, ¹H NMR (400 MHz, Methanol-d₄) δ 8.59 (s, 1H), 8.52 (d, J = 8.2 Hz, 1H), 8.32 (s, 1H), 8.28 (d, J = 1.7 Hz, 1H), 7.93 (s, 1H), 7.72 (d, J = 1.6 Hz, 1H), 7.66 (dd, J = 8.2, 1.6 Hz, 1H), 4.06 (s, 3H), 3.78 (m, 2H), 3.45 (m, 2H), 1.81-1.69 (m, 4H), 1.60 (m, 2H) |
| 54 | 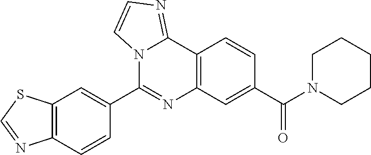 [SMILES: O=C(c1ccc2c(c1)nc(-c1ccc3ncsc3c1)n1ccnc21)N1CCCCC1] | Method III, Example 46, Preparation Example 11 | MS (ESI) m/z (M + H)⁺ = 414.1, ¹H NMR (400 MHz, Methanol-d₄) δ 9.47 (s, 1H), 8.77 (d, J = 1.7 Hz, 1H), 8.60 (d, J = 8.2 Hz, 1H), 8.34 (d, J = 8.5 Hz, 1H), 8.17 (dd, J = 8.5, 1.7 Hz, 1H), 8.02 (s, 2H), 7.75 (dd, J = 8.3, 1.6 Hz, 1H), 7.69 (d, J = 1.6 Hz, 1H), 3.78 (m, 2H), 3.47 (m, 2H), 1.74 (m, 4H), 1.61 (m, 2H) |
| 55 | 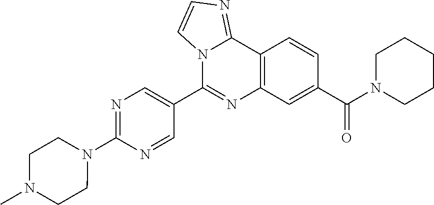 [SMILES: CN1CCN(c2ncc(-c3nc4cc(C(=O)N5CCCCC5)ccc4c4nccn34)cn2)CC1] | Method III, Example 46, Preparation Example 11 | MS (ESI) m/z (M + H)⁺ = 457.2, ¹H NMR (400 MHz, Methanol-d₄) δ 8.99 (s, 2H), 8.53 (d, J = 8.2 Hz, 1H), 8.10 (d, J = 1.8 Hz, 1H), 8.00-7.93 (m, 1H), 7.72-7.65 (m, 2H), 4.01 (t, J = 5.0 Hz, 4H), 3.77 (m, 2H), 3.45 (m, 2H), 2.56 (t, J = 5.0 Hz, 4H), 2.37 (s, 3H), 1.74 (m, 4H), 1.60 (m, 2H) |
| 56 | 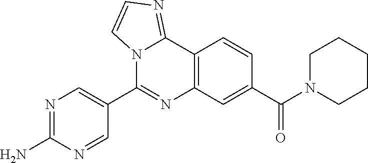 [SMILES: Nc1ncc(-c2nc3cc(C(=O)N4CCCCC4)ccc3c3nccn23)cn1] | Method III, Example 47, Preparation Example 11 | MS (ESI) m/z (M + H)⁺ = 374.1, ¹H NMR (400 MHz, DMSO-d₆) δ 8.87 (s, 2H), 8.45 (d, J = 8.1 Hz, 1H), 8.19 (d, J = 1.5 Hz, 1H), 7.87 (d, J = 1.5 Hz, 1H), 7.69 (d, J = 1.5 Hz, 1H), 7.65 (dd, J = 8.1, 1.6 Hz, 1H), 7.44 (s, 2H), 3.64 (m, 2H), 3.33 (m, 2H), 1.57 (m, 6H) |
| 57 | 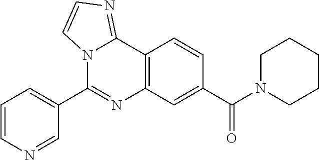 [SMILES: O=C(c1ccc2c(c1)nc(-c1cccnc1)n1ccnc21)N1CCCCC1] | Method III, Example 46, Preparation Example 11 | MS (ESI) m/z (M + H)⁺ = 358.1, ¹H NMR (400 MHz, Methanol-d₄) δ 9.18 (dd, J = 2.4, 0.8 Hz, 1H), 8.85 (dd, J = 5.0, 1.6 Hz, 1H), 8.60 (d, J = 8.2 Hz, 1H), 8.49 (dt, J = 8.0, 2.0 Hz, 1H), 8.03 (d, J = 1.6 Hz, 1H), 8.00 (d, J = 1.6 Hz, 1H), 7.78-7.70 (m, 3H), 3.78 (m, 2H), 3.46 (m, 2H), 1.74 (m, 4H), 1.60 (m, 2H) |
| 58 | 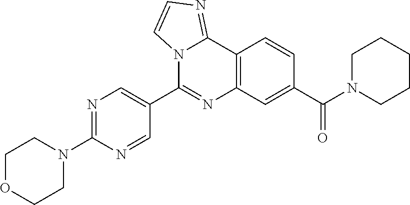 [SMILES: O=C(c1ccc2c(c1)nc(-c1cnc(N3CCOCC3)nc1)n1ccnc21)N1CCCCC1] | Method III, Example 46, Preparation Example 11 | MS (ESI) m/z (M + H)⁺ = 444.2, ¹H NMR (400 MHz, Methanol-d₄) δ 9.01 (s, 2H), 8.55 (d, J = 8.1 Hz, 1H), 8.11 (d, J = 1.6 Hz, 1H), 7.98 (d, J = 1.5 Hz, 1H), 7.72-7.68 (m, 2H), 4.00-3.96 (m, 4H), 3.81-3.74 (m, 6H), 3.45 (m, 2H), 1.74 (m, 4H), 1.60 (m, 2H) |
| 59 | 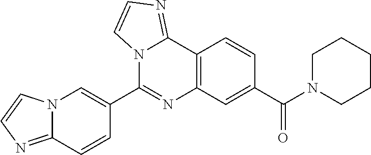 [SMILES: O=C(c1ccc2c(c1)nc(-c1ccc3nccn3c1)n1ccnc21)N1CCCCC1] | Method III, Example 46, Preparation Example 11 | MS (ESI) m/z (M + H)⁺ = 397.2, ¹H NMR (400 MHz, Methanol-d₄) δ 9.26 (t, J = 1.3 Hz, 1H), 8.58 (d, J = 8.2 Hz, 1H), 8.14 (d, J = 1.6 Hz, 1H), 8.06 (d, J = 1.3 Hz, 1H), 8.01 (d, J = 1.5 Hz, 1H), 7.89 (dd, J = 9.4, 1.8 Hz, 1H), 7.80 (d, J = 9.4 Hz, 1H), 7.77-7.73 (m, 2H), 7.71 (d, J = 1.6 Hz, 1H), 3.77 (m, 2H), 3.46 (m, 2H), 1.79-1.69 (m, 4H), 1.60 (m, 2H) |
| 60 | 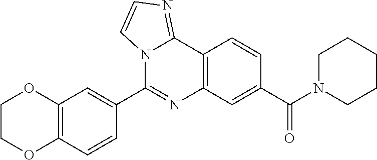 [SMILES: O=C(c1ccc2c(c1)nc(-c1ccc3c(c1)OCCO3)n1ccnc21)N1CCCCC1] | Method III, Example 46, Preparation Example 11 | MS (ESI) m/z (M + H)⁺ = 415.1, ¹H NMR (400 MHz, Methanol-d₄) δ 8.55 (d, J = 8.2 Hz, 1H), 8.00 (d, J = 1.6 Hz, 1H), 7.97 (d, J = 1.5 Hz, 1H), 7.71 (dd, J = 8.2, 1.6 Hz, 1H), 7.66 (d, J = 1.6 Hz, 1H), 7.51-7.46 (m, 2H), 7.10 (d, J = 8.2 Hz, 1H), 4.40-4.33 (m, 4H), 3.77 (m, 2H), 3.46 (m, 2H), 1.74 (m, 4H), 1.60 (m, 2H) |
| 61 | 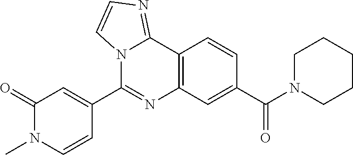 [SMILES: Cn1ccc(-c2nc3cc(C(=O)N4CCCCC4)ccc3c3nccn23)cc1=O] | Method III, Example 46, Preparation Example 11 | MS (ESI) m/z (M + H)⁺ = 388.2, ¹H NMR (400 MHz, Methanol-d₄) δ 8.59 (d, J = 8.2 Hz, 1H), 8.02 (t, J = 1.9 Hz, 2H), 7.93 (d, J = 7.0 Hz, 1H), 7.77 (dd, J = 8.2, 1.6 Hz, 1H), 7.70 (d, J = 1.6 Hz, 1H), 7.13 (d, J = 1.9 Hz, 1H), 6.87 (dd, J = 7.0, 2.0 Hz, 1H), 3.78 (m, 2H), 3.69 (s, 3H), 3.45 (m, 2H), 1.74 (m, 4H), 1.60 (m, 2H) |
| 62 | 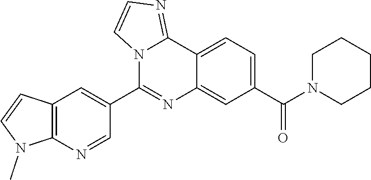 [SMILES: Cn1ccc2cc(-c3nc4cc(C(=O)N5CCCCC5)ccc4c4nccn34)cnc21] | Method III, Example 46, Preparation Example 11 | MS (ESI) m/z (M + H)⁺ = 411.2, ¹H NMR (400 MHz, DMSO-d₆) δ 8.85 (d, J = 2.0 Hz, 1H), 8.62 (d, J = 2.0 Hz, 1H), 8.51- 8.49 (d, J = 8.1 Hz, 1H), 8.07 (s, 1H), 7.91 (s, 1H), 7.72 (d, J = 3.5 Hz, 1H), 7.71-7.66 (m, 2H), 6.69-6.68 (d, J = 3.5 Hz, 1H), 3.93 (s, 3H), 3.65 (m, 2H), 3.36 (m, 2H), 1.70-1.48 (m, 6H) |
| 63 | 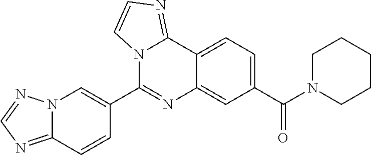 [SMILES: O=C(c1ccc2c(c1)nc(-c1ccc3ncnn3c1)n1ccnc21)N1CCCCC1] | Method III, Example 46, Preparation Example 11 | MS (ESI) m/z (M + H)⁺ = 398.1, ¹H NMR (400 MHz, DMSO-d₆) δ 9.65 (d, J = 1.7 Hz, 1H), 8.71 (s, 1H), 8.53-8.51 (d, J = 8.1 Hz, 1H), 8.20-8.16 (m, 2H), 8.12-8.09 (d, J = 9.2 Hz, 1H), 7.94 (d, J = 1.6 Hz, 1H), 7.74-7.71 (dd, J = 10.2, 1.7 Hz, 2H), 3.63 (m, 2H), 3.35 (m, 2H), 1.66-1.52 (m, 6H) |
| 64 | 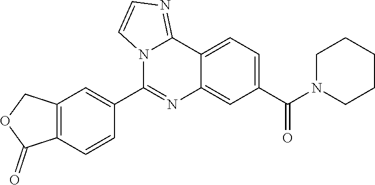 [SMILES: O=C1OCc2cc(-c3nc4cc(C(=O)N5CCCCC5)ccc4c4nccn34)ccc21] | Method III, Example 46, Preparation Example 11 | MS (ESI) m/z (M + H)⁺ = 413.1, ¹H NMR (400 MHz, DMSO-d₆) δ 8.53-8.51 (d, J = 8.1 Hz, 1H), 8.27 (s, 1H), 8.17-8.15 (d, J = 8.0 Hz, 1H), 8.12-8.10 (d, J = 8.0 Hz, 1H), 7.99 (d, J = 1.5 Hz, 1H), 7.93 (m, 1H), 7.74-7.71 (m, 2H), 5.57 (s, 2H), 3.65 (m, 2H), 3.37 (m, 2H), 1.66-1.51 (m, 6H) |
| 65 | 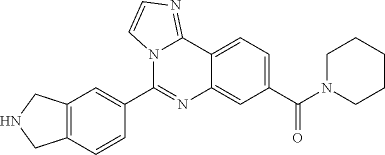 [SMILES: O=C(c1ccc2c(c1)nc(-c1ccc3c(c1)CNC3)n1ccnc21)N1CCCCC1] | Method III, Example 47, Preparation Example 11 | MS (ESI) m/z (M + H)⁺ = 398.1, ¹H NMR (400 MHz, DMSO-d₆) δ 8.48 (d, J = 8.1 Hz, 1H), 7.97 (s, 1H), 7.89 (s, 1H), 7.85 (s, 1H), 7.81-7.79 (d, J = 7.8 Hz, 1H), 7.69-7.67 (m, 2H), 7.54-7.52 (d, J = 7.8 Hz, 1H), 4.20 (s, 4H), 3.64 (m, 2H), 3.36 (m, 2H), 1.66-1.51 (m, 6H) |
| 66 | 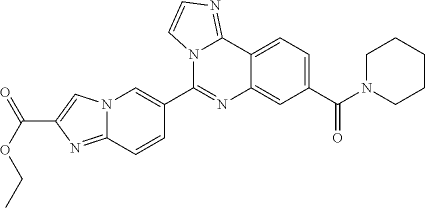 [SMILES: CCOC(=O)c1cn2cc(-c3nc4cc(C(=O)N5CCCCC5)ccc4c4nccn34)ccc2n1] | Method III, Example 46, Preparation Example 11 | MS (ESI) m/z (M + H)⁺ = 469.1, ¹H NMR (400 MHz, DMSO-d₆) δ 9.36 (s, 1H), 8.72 (s, 1H), 8.52-8.50 (d, J = 8.1 Hz, 1H), 8.26 (s, 1H), 7.93-7.85 (m, 3H), 7.75- 7.71 (m, 2H), 4.39-4.33 (q, J = 7.1 Hz, 2H), 3.65 (m, 2H), 3.34 (m, 2H), 1.66-1.51 (m, 6H), 1.37-1.33 (t, J = 7.1 Hz, 3H) |
| 67 | 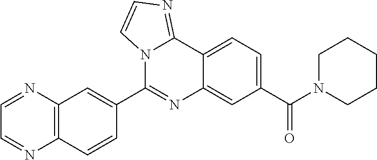 [SMILES: O=C(c1ccc2c(c1)nc(-c1ccc3nccnc3c1)n1ccnc21)N1CCCCC1] | Method III, Example 46, Preparation Example 11 | MS (ESI) m/z (M + H)⁺ = 409.2, ¹H NMR (400 MHz, Methanol-d₄) δ 9.02 (s, 2H), 8.75 (d, J = 1.8 Hz, 1H), 8.60 (d, J = 8.2 Hz, 1H), 8.42 (dd, J = 8.7, 1.9 Hz, 1H), 8.35 (d, J = 8.7 Hz, 1H), 8.07 (d, J = 1.6 Hz, 1H), 8.05 (d, J = 1.6 Hz, 1H), 7.77 (dd, J = 8.2, 1.6 Hz, 1H), 7.71 (d, J = 1.6 Hz, 1H), 3.78 (m, 2H), 3.55-3.42 (m, 2H), 1.74 (m, 4H), 1.61 (m, 2H) |
| 68 | 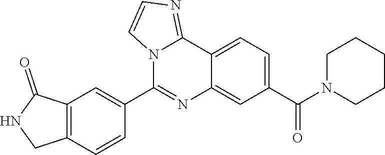 [SMILES: O=C1NCc2ccc(-c3nc4cc(C(=O)N5CCCCC5)ccc4c4nccn34)cc21] | Method III, Example 46, Preparation Example 11 | MS (ESI) m/z (M + H)⁺ = 412.2, ¹H NMR (400 MHz, DMSO-d₆) δ 8.81 (s, 1H), 8.50 (d, J = 8.1 Hz, 1H), 8.24-8.17 (m, 2H), 7.95 (d, J = 1.5 Hz, 1H), 7.92 (d, J = 1.5 Hz, 1H), 7.87 (d, J = 7.8 Hz, 1H), 7.73-7.68 (m, 2H), 4.55 (s, 2H), 3.65 (m, 2H), 3.33(m, 2H), 1.57 (m, 6H) |
| 69 | 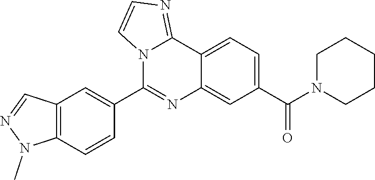 [SMILES: Cn1ncc2cc(-c3nc4cc(C(=O)N5CCCCC5)ccc4c4nccn34)ccc21] | Method III, Example 46, Preparation Example 11 | MS (ESI) m/z (M + H)⁺ = 411.2, ¹H NMR (400 MHz, Methanol-d₄) δ 8.57 (d, J = 8.2 Hz, 1H), 8.48-8.43 (m, 1H), 8.20 (s, 1H), 8.00 (ddd, J = 6.6, 4.3, 1.7 Hz, 3H), 7.81 (d, J = 8.8 Hz, 1H), 7.72 (dd, J = 8.2, 1.6 Hz, 1H), 7.67 (d, J = 1.6 Hz, 1H), 4.17 (s, 3H), 3.78 (m, 2H), 3.46 (m, 2H), 1.79-1.68 (m, 4H), 1.60 (m, 2H) |
| 70 | 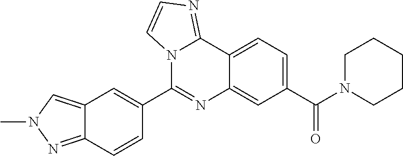 [SMILES: Cn1cc2cc(-c3nc4cc(C(=O)N5CCCCC5)ccc4c4nccn34)ccc2n1] | Method III, Example 46, Preparation Example 11 | MS (ESI) m/z (M + H)⁺ = 412.2, ¹H NMR (400 MHz, Methanol-d₄) δ 8.57 (d, J = 8.2 Hz, 1H), 8.47-8.41 (m, 2H), 8.01 (dd, J = 12.0, 1.6 Hz, 2H), 7.85 (d, J = 1.6 Hz, 2H), 7.72 (dd, J = 8.2, 1.6 Hz, 1H), 7.67 (d, J = 1.6 Hz, 1H), 4.29 (s, 3H), 3.78 (m, 2H), 3.46 (m, 2H), 1.74 (m, 4H), 1.60 (m, 2H) |
| 71 | 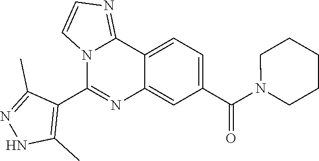 [SMILES: Cc1n[nH]c(C)c1-c1nc2cc(C(=O)N3CCCCC3)ccc2c2nccn12] | Method III, Example 47, Preparation Example 11 | MS (ESI) m/z (M + H)⁺ = 375.2, ¹H NMR (400 MHz, DMSO-d₆) δ 12.85 (s, 1H), 8.46 (d, J = 8.1 Hz, 1H), 7.84 (d, J = 1.5 Hz, 1H), 7.75 (d, J = 1.5 Hz, 1H), 7.70-7.61 (m, 2H), 3.64 (m, 2H), 3.45 (m, 2H), 2.27 (s, 3H), 2.19 (s, 3H), 1.57 (m, 6H) |
| 72 | 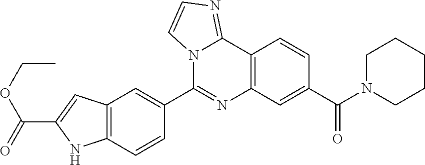 [SMILES: CCOC(=O)c1cc2cc(-c3nc4cc(C(=O)N5CCCCC5)ccc4c4nccn34)ccc2[nH]1] | Method III, Example 46, Preparation Example 11 | MS (ESI) m/z (M + H)⁺ = 468.2, ¹H NMR (400 MHz, Methanol-d₄) δ 8.52 (dd, J = 8.3, 2.8 Hz, 1H), 8.28 (dd, J = 4.1, 1.9 Hz, 1H), 8.01-7.93 (m, 2H), 7.82 (ddd, J = 8.7, 3.2, 1.7 Hz, 1H), 7.72-7.59 (m, 3H), 7.32-7.26 (m, 1H), 4.41 (q, J = 7.1 Hz, 2H), 3.76 (m, 2H), 3.45 (m, 2H), 1.73 (m, 4H), 1.59 (m, 2H), 1.42 (t, J = 7.1 Hz, 3H) |
| 73 | 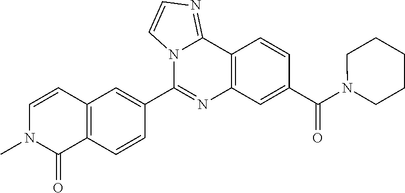 [SMILES: Cn1ccc2cc(-c3nc4cc(C(=O)N5CCCCC5)ccc4c4nccn34)ccc2c1=O] | Method III, Example 46, Preparation Example 11 | MS (ESI) m/z (M + H)⁺ = 438.2, ¹H NMR (400 MHz, Methanol-d₄) δ 8.56 (d, J = 8.2 Hz, 1H), 8.51 (d, J = 8.4 Hz, 1H), 8.24 (d, J = 1.7 Hz, 1H), 8.04 (dd, J = 8.4, 1.7 Hz, 1H), 8.00 (d, J = 1.6 Hz, 1H), 7.97 (d, J = 1.6 Hz, 1H), 7.74 (dd, J = 8.2, 1.6 Hz, 1H), 7.67 (d, J = 1.6 Hz, 1H), 7.47 (d, J = 7.4 Hz, 1H), 6.79 (d, J = 7.3 Hz, 1H), 3.77 (m, 2H), 3.65 (s, 3H), 3.47 (m, 2H), 1.74 (m, 4H), 1.60 (m, 2H) |
| 74 | 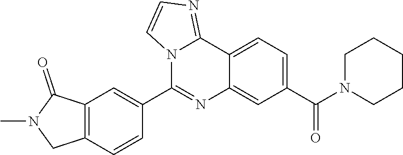 [SMILES: CN1Cc2ccc(-c3nc4cc(C(=O)N5CCCCC5)ccc4c4nccn34)cc2C1=O] | Method III, Example 46, Preparation Example 11 | MS (ESI) m/z (M + H)⁺ = 426.2, ¹H NMR (400 MHz, Methanol-d₄) δ 8.59 (d, J = 8.2 Hz, 1H), 8.35 (s, 1H), 8.23 (dd, J = 7.9, 1.6 Hz, 1H), 8.02 (d, J = 1.5 Hz, 1H), 7.97 (d, J = 1.6 Hz, 1H), 7.86 (d, J = 7.9 Hz, 1H), 7.75 (dd, J = 8.3, 1.6 Hz, 1H), 7.69 (d, J = 1.6 Hz, 1H), 4.67 (s, 2H), 3.78 (m, 2H), 3.46 (m, 2H), 3.26 (s, 3H), 1.74 (m, 4H), 1.61 (m, 2H) |
| 75 | 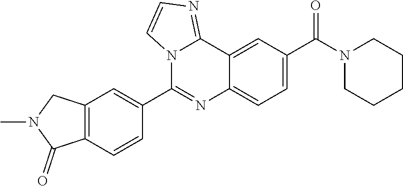 [SMILES: CN1Cc2cc(-c3nc4ccc(C(=O)N5CCCCC5)cc4c4nccn34)ccc2C1=O] | Method III, Example 46, Preparation Example 19 | MS (ESI) m/z (M + H)⁺ = 426.2 ¹H NMR (400 MHz, DMSO-d₆) δ 8.39 (d, J = 1.9 Hz, 1H), 8.19 (s, 1H), 8.09-8.02 (m, 2H), 8.00 (d, J = 1.5 Hz, 1H), 7.92 (d, J = 7.9 Hz, 1H), 7.76 (dd, J = 8.3, 1.9 Hz, 1H), 7.70 (d, J = 1.5 Hz, 1H), 4.62 (s, 2H), 3.66 (m, 2H), 3.36 (m, 2H),3.15 (s, 3H), 1.59 (d, J = 51.7 Hz, 6H). |
| 76 | 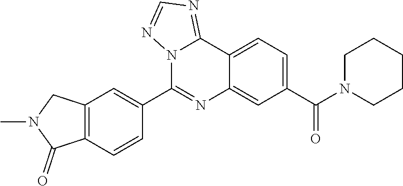 [SMILES: CN1Cc2cc(-c3nc4cc(C(=O)N5CCCCC5)ccc4c4ncnn34)ccc2C1=O] | Method III, Example 46, Preparation Example 13 | MS (ESI) m/z (M + H)⁺ = 427.1, ¹H NMR (400 MHz, DMSO-d6) δ 8.82 (s, 1H), 8.65 (s, 1H), 8.59-8.55 (m, 2H), 8.10 (m, 1H), 7.95-7.93 (d, J = 8.0 Hz, 1H), 7.84- 7.82 (d, J = 8.0 Hz, 1H), 4.65 (s, 2H), 3.68 (s, 2H), 3.30 (m, 2H), 3.15 (s, 3H), 1.65 (s, 4H), 1.52 (s, 2H) |
| 77 | 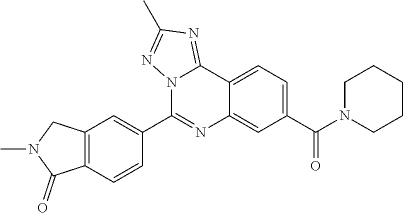 [SMILES: Cc1nc2c3ccc(C(=O)N4CCCCC4)cc3nc(-c3ccc4c(c3)CN(C)C4=O)n2n1] | Method III, Example 46, Preparation Example 14 | MS (ESI) m/z (M + H)⁺ = 441.2, ¹H NMR (400 MHz, DMSO-d6) δ 8.61 (s, 1H), 8.56-8.50 (m, 2H), 8.05 (s, 1H), 7.93- 7.91 (d, J = 8.0 Hz, 1H), 7.80-7.78 (m, 1H), 4.64 (s, 2H), 3.67 (s, 2H), 3.30 (s, 2H), 3.15 (s, 3H), 2.63 (s, 3H), 1.64 (s, 4H), 1.51 (s, 2H) |
| 78 | 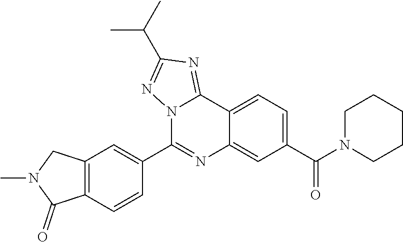 [SMILES: CC(C)c1nc2c3ccc(C(=O)N4CCCCC4)cc3nc(-c3ccc4c(c3)CN(C)C4=O)n2n1] | Method III, Example 46, Preparation Example 15 | MS (ESI) m/z (M + H)⁺ = 469.3, ¹H NMR (400 MHz, DMSO-d6) δ 8.62 (s, 1H), 8.58-8.55 (m, 1H), 8.52-8.49 (d, J = 8.4 Hz, 1H), 8.033-8.029 (d, J = 1.6 Hz, 1H), 7.90-7.88 (d, J = 8.0 Hz, 1H), 7.77-7.74 (m, 1H), 4.69-4.56 (m, 2H), 3.65-3.58 (d, J = 30.0 Hz, 2H), 3.34-3.30 (d, J = 13.6 Hz, 1H), 3.30-3.19 (m, 2H), 3.13 (s, 3H), 1.68- 1.47 (m, 6H), 1.43-1.41 (d, J = 7.2 Hz, 6H) |
| 79 | 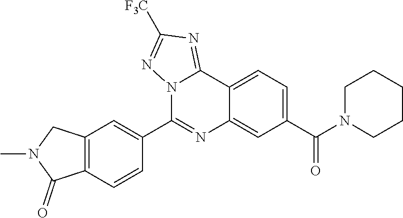 [SMILES: CN1Cc2cc(-c3nc4cc(C(=O)N5CCCCC5)ccc4c4nc(C(F)(F)F)nn34)ccc2C1=O] | Method III, Example 46, Preparation Example 16 | MS (ESI) m/z (M + H)⁺ = 495.1, ¹H NMR (400 MHz, DMSO-d6) δ 8.65-8.63 (d, J = 8.2 Hz, 1H), 8.51 (s, 1H), 8.46-8.44 (d, J = 8.0 Hz, 1H), 8.16 (d, J = 0.8 Hz, 1H), 7.97-7.95 (d, J = 8.0 Hz, 1H), 7.89-7.86 (m, 1H), 4.66 (s, 2H), 3.68 (s, 2H), 3.15 (s, 3H), 1.64 (s, 6H), 1.51 (s, 2 H) |
| 80 | 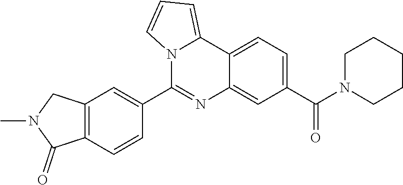 [SMILES: CN1Cc2cc(-c3nc4cc(C(=O)N5CCCCC5)ccc4c4cccn34)ccc2C1=O] | Method III, Example 46, Preparation Example 17 | MS (ESI) m/z (M + H)⁺ = 425.2, ¹H NMR (400 MHz, DMSO-d6) δ 8.25-8.23 (d, J = 8.0 Hz, 1H), 8.11 (s, 1H), 7.99-7.97 (d, J = 8.0 Hz, 1H), 7.90-7.88 (d, J = 4.0 Hz, 1H), 7.72-7.71 (d, J = 1.6 Hz, 1H), 7.50-7.48 (m, 2H), 7.32-7.31 (d, J = 4.0 Hz, 1H), 6.90- 6.89 (m, 1H), 4.60 (s, 2H), 3.70-3.36 (m, 4H), 3.14 (s, 3H), 1.63-1.53 (d, J = 40.0 Hz, 6H) |
| 81 | 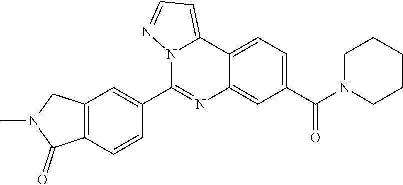 [SMILES: CN1Cc2cc(-c3nc4cc(C(=O)N5CCCCC5)ccc4c4ccnn34)ccc2C1=O] | Method III, Example 46, Preparation Example 18 | MS (ESI) m/z (M + H)⁺ = 426.3, ¹H NMR (400 MHz, DMSO-d6) δ 8.47 (s, 1H), 8.38-8.34 (t, J = 14.8 Hz, 2H), 8.26 (s, 1H), 7.86 (s, 1H), 7.83-7.81 (d, J = 0.8 Hz, 1H), 7.64-7.62 (d, J = 0.8 Hz, 1H), 7.48 (s, 1H), 4.56 (s, 2H), 3.58 (s, 2H), 3.24-3.20 (m, 2H), 3.08 (s, 3H), 1.57-1.49 (m, 6H) |
| 82 | 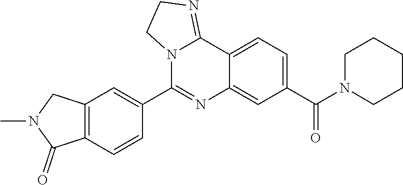 [SMILES: CN1Cc2cc(C3=Nc4cc(C(=O)N5CCCCC5)ccc4C4=NCCN34)ccc2C1=O] | Method III, Example 46, Preparation Example 12 | MS (ESI) m/z (M + H)⁺ = 427.9, ¹H NMR (400 MHz, DMSO-d6) δ 7.96-7.90 (m, 2H), 7.85-7.75 (m, 2H), 7.36 (s, 1H), 7.33-7.30 (m, 1H), 4.55 (s, 2H), 4.05-3.95 (m, 4H), 3.67-3.53 (m, 2H), 3.30-3.18 (m, 2H), 3.11 (s, 3H), 1.67-1.39 (m, 6H) |

### Biological Tests

### Test Example 1: Detection of Activity of 15-PGDH Kinase

### 1. Experimental Materials

| Reagents/Materials/Instruments | Manufacturer | Item No./Model No. |
|---|---|---|
| 15-PGDH | Sino Biological Inc. | 11205-H08E |
| β-NAD | Sigma-Aldrich Corporation | N6522 |
| PGF2α | MedChemExpress LLC | HY-12956A |
| DMSO | Sigma-Aldrich Corporation | D8418 |
| 384-well plate | Corning United States Corporation | 4513 |
| Tween 20 | Shanghai Macklin Biochemical | T818927 |
| | Technology Co., Ltd. | |
| Tris-HCl | Shanghai Beyotime Biotech Inc. | ST774 |
| Multifunctional microplate reader | BMG LABTECH Corporation | PHERAstar ® FSX |

### 2. Experimental Method

a. A solution of pH 7.5 containing 50 mM Tris-HCl and 0.01% Tween 20 was prepared with ultrapure water as a reaction buffer;
b. a 10 mM mother liquor of the compound to be tested was prepared with DMSO, and then the reaction buffer was used to dilute the mother liquor of the compound to be tested to obtain solution 1 of the compound to be tested at a concentration of 4000 nM, and then the solution 1 of the compound to be tested was serially diluted into solutions 2-10 of the compound to be tested at 9 concentrations with a gradient difference of three-fold. 5 μL of each concentration of solutions 1-10 of the compound to be tested was respectively taken and added into a 384-well plate as test wells;
c. 5 μL of the reaction buffer was added to the 384-well plate as positive control and blank control wells;
d. the reaction buffer was used to prepare a 15-PGDH protein solution at a concentration of 5 ng/μL, 5 μL of the 15-PGDH protein solution was taken and added to the test wells and positive control wells, and 5 μL of the reaction buffer was added to the blank control wells at the same time, then the plate was centrifuged at 2000 rpm for 30 seconds;
e. the reaction buffer was used to prepare 5 mM β-NAD and 2 mM PGF2α, respectively, which were mixed at 1:1 by volume to obtain a substrate mixture, 10 μL of the substrate mixture was taken and added to the test wells, positive control wells and blank control wells to start the reaction;
f. the fluorescence signal value (Ex/Em=340/450) of each well was detected continuously by using a multifunctional microplate reader.

### 3. Data Analysis

a) Continuous fluorescence signal values were analyzed by using the “kinetic calculations-slope calculation method” in the PHERAstar Data analysis software to obtain the slope of each test well;
b) the inhibition rate % was calculated by using the following formula:

inhibition rate %=[1−(slope of test well-signal value of positive control well)/(signal value of blank control well-average signal value of positive control well)]×100%.
c) calculation of IC₅₀ and plotting of inhibition rate-dose curves: IC₅₀ values were calculated by fitting compound concentrations and corresponding inhibition rates with a nonlinear regression (dose response-variable slope) via using GraphPad Prism 6.0.

The formula is shown below: Y=Bottom+(Top−Bottom)/(1+10\hat{ }((Log IC50−X)*HillSlope)), wherein X is a log value of a concentration of the compound, and Y is inhibition rate %.

### 4. Experimental Results

Inhibitory activities of the compounds in the present application against the 15-PGDH enzyme are shown in Table 3 below.

TABLE 3

| Example No. | IC₅₀ (nM) |
|---|---|
| Example 2 | B |
| Example 3 | B |
| Example 4 | A |
| Example 5 | C |
| Example 6 | A |
| Example 7 | B |
| Example 8 | C |
| Example 10 | C |
| Example 16 | G |
| Example 17 | G |
| Example 18 | D |
| Example 19 | C |
| Example 20 | A |
| Example 21 | A |
| Example 23 | B |
| Example 24 | B |
| Example 27 | C |
| Example 29 | A |
| Example 30 | C |
| Example 31 | C |
| Example 33 | A |
| Example 34 | A |
| Example 35 | A |
| Example 36 | A |
| Example 37 | A |
| Example 38 | B |
| Example 39 | E |
| Example 40 | F |
| Example 41 | A |
| Example 42 | C |
| Example 43 | C |
| Example 44 | C |
| Example 45 | C |
| Example 46 | B |
| Example 47 | B |
| Example 50 | C |
| Example 52 | C |
| Example 53 | B |
| Example 54 | A |
| Example 56 | A |
| Example 57 | B |
| Example 59 | C |
| Example 60 | C |
| Example 61 | C |
| Example 62 | A |
| Example 67 | B |
| Example 69 | B |
| Example 70 | B |
| Example 72 | C |
| Example 73 | A |
| Example 75 | G |
| Example 76 | A |
| Example 77 | A |
| Example 78 | B |
| Example 79 | B |
| Example 80 | A |
| Example 81 | A |
| Example 82 | C |

In the table, “A” represents that IC₅₀ of the inhibitory activity against 15-PGDH enzyme is in a range of less than or equal to 2 nM; “B” represents that IC₅₀ of the inhibitory activity against 15-PGDH enzyme is in a range of greater than 2 nM and less than or equal to 5 nM; “C” represents that IC₅₀ of the inhibitory activity against 15-PGDH enzyme is in a range of greater than 5 nM and less than or equal to 10 nM; “D” represents that IC₅₀ of the inhibitory activity against 15-PGDH enzyme is in a range of greater than 10 nM and less than or equal to 20 nM; “E” represents that IC₅₀ of the inhibitory activity against 15-PGDH enzyme is in a range of greater than 20 nM and less than or equal to 50 nM; “F” represents that IC₅₀ of the inhibitory activity against 15-PGDH enzyme is in a range of greater than 50 nM and less than or equal to 100 nM; “G” represents that IC₅₀ of the inhibitory activity against 15-PGDH enzyme is in a range of greater than 100 nM and less than or equal to 1000 nM.

It was found via the tests that the IC₅₀ value of the inhibitory activity of the compounds in the present application against 15-PGDH enzyme is less than 100 nM; the IC₅₀ value of the inhibitory activity of some compounds in the present application against 15-PGDH enzyme is greater than or equal to 20 nM and less than 50 nM; the IC₅₀ value of the inhibitory activity of some compounds in the present application against 15-PGDH enzyme is greater than or equal to 10 nM and less than 20 nM; the IC₅₀ value of the inhibitory activity of some compounds in the present application against 15-PGDH enzyme is greater than or equal to 5 nM and less than 10 nM; the IC₅₀ value of the inhibitory activity of some compounds in the present application against 15-PGDH enzyme is greater than or equal to 2 nM and less than 5 nM; the IC₅₀ value of the inhibitory activity of some compounds in the present application against 15-PGDH enzyme is less than 2 nM.

### Test Example 2: Assay of Intracellular PGE2 Up-Regulatory Activity

### 1. Experimental Materials

| Reagents/Materials/Instruments | Manufacturer | Item No. |
|---|---|---|
| F12k Kaighn's Modification culture | Hyclone Laboratories, Inc | SH3052601/AG29722854 |
| medium | | |
| TRYPSIN | Hyclone Laboratories, Inc | J190002 |
| Fetal Bovine Serum | PAN-Biotech | ST-30-3302 |
| Penicillin-Streptomycin | Hyclone Laboratories, Inc | J190007 |
| DMSO | Sigma-Aldrich Corporation | D8418 |
| A549 cells | Nanjing Cobioer Biosciences | CBP60084 |
| | Co., Ltd. | |
| Prostaglandin E2 Kit | PerkinElmer Corporation | 62P2APEG |
| 24-well plate | Corning United States | 3337 |
| | Corporation | |
| 384-well plate | Corning United States | 3570 |
| | Corporation | |
| Multifunctional microplate reader | BMG LABTECH Corporation | PHERAstar ® FSX |
| CO₂ cell incubator | Thermo Fisher Scientific | RI-250 |
| | Corporation | |
| Microscope | Thermo Fisher Scientific | DMI1 |
| | Corporation | |

### 2. Experimental Method

a) A549 cells were inoculated in the 24-well plate, and after cell adhesion, IL-1β was added thereto for 16 h of stimulation to induce COX2 expression and PGE2 production;
b) the compounds of Examples 1-82 were each respectively prepared into a compound solution to be tested with F12k Kaighn's Modification culture medium and serially diluted to 7 concentrations of 0.64 nM, 3.2 nM, 16 nM, 80 nM, 400 nM, 2000 nM, and 10000 nM, and meanwhile the positive control group and negative control group were set up; the cell supernatants were collected after 8 h of action, in which the positive control group was induced by IL-1B without treatment of the compounds to be tested, and the negative control group was neither stimulated by IL-1B, nor treated with the compounds to be tested;
c) the PGE2 content of the samples was determined by Prostaglandin E2 Kit, and the fluorescence signal was detected by a multifunctional microplate reader (Ex/Em=337/620, 337/665).

### 3. Data Analysis

a) A standard curve was plotted with the PGE2 standard in the Prostaglandin E2 Kit, and the PGE2 concentration was calculated by substituting with the fluorescence signal of the sample;
b) the PGE2 up-regulation rate % was calculated by using the following formula:

PGE2 up-regulation rate %−(PGE2 concentration of sample group/PGE2 concentration of positive control group)×100%.

### 4. Experimental Results

The compounds of the present application, particularly the compounds prepared in Examples, are able to achieve a PGE2 up-regulation rate of greater than 100% in A549 cells. The compounds of the present application, particularly the compounds prepared in Examples, can have intracellular PGE2 up-regulatory activity.

For the purpose of describing and disclosing, all patents, patent applications and other established publications are expressly incorporated herein by reference. These publications are provided solely for their disclosure prior to the filing date of this application. All statements regarding the dates of these documents or the representation of the contents of these documents are based on the information available to the applicants and do not constitute any admission as to the correctness of the dates of these documents or the contents of these documents. Moreover, any reference to these publications herein does not constitute an admission that the publications form part of the common general knowledge in the art in any country.

The skilled in the art will recognize that the scope of the present application is not limited to the various specific embodiments and examples described above, but is capable of making various modifications, substitutions, or recombinations without departing from the spirit of the present application, and that these adjusted technical solutions fall within the protection scope of the present application.

1. A compound represented by formula (I), a stereoisomer thereof, a tautomer thereof or a mixture form thereof, or a pharmaceutically acceptable salt thereof, or a solvate thereof, or a prodrug thereof, 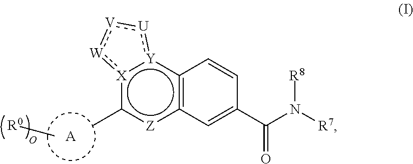
wherein ring A is selected from the group consisting of an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle;
o is selected from the group consisting of 0, 1, 2, 3, and 4,
R⁰ is each independently selected from the group consisting of deuterium, tritium, nitro, hydroxyl, mercapto, halogen, cyano, ═O, imino, an amine group, an ester group, an aldehyde group, carboxyl, carbonyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl,
◯ represents an aromatic ring, [symbol] is a single bond or double bond,
Z is N or CH, X and Y are each independently C or N, and at least one of X, Y, and Z is N,
U, V, and W are each independently selected from the group consisting of N, CR², CR³R⁴, NR⁵, O, and S, wherein the R², R³, R⁴, and R⁵ are each independently selected from the group consisting of hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl, or the R³ and the R⁴, together with the carbon atom to which they are attached, form C═O, C═NR^{W}, or ring B, wherein the R^{W} is hydrogen, deuterium, tritium, C₁-C₆ alkyl, or 3-12 membered cycloalkyl, and the ring B is 3-12 membered cycloalkyl or 3-12 membered heterocycloalkyl, R⁷ and R⁸ are each independently C₁-C₁₀ chain hydrocarbyl, or R⁷ and R⁸, together with the nitrogen atom to which they are attached, form 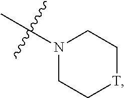
the T is a covalent bond, O, or CR⁹R¹⁰, and the R⁹ and R¹⁰ are each independently selected from the group consisting of hydrogen, deuterium, tritium, nitro, hydroxyl, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, and C₁-C₆ haloalkoxy,
wherein R⁰, R², R³, R⁴, R⁵, and ring B are optionally substituted by one or two or more independently selected from the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl, the aromatic heterocycle, the aliphatic heterocycle, the unsaturated aliphatic heterocycle, the fused ring, and the heterocycloalkyl each independently comprise 1-3 heteroatoms which are independently N, O, or S.

2. The compound, the stereoisomer thereof, the tautomer thereof or the mixture form thereof, or the pharmaceutically acceptable salt thereof, or the solvate thereof, or the prodrug thereof according to claim 1, wherein the 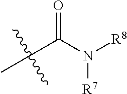
is 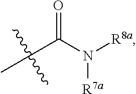
wherein the R^{7a} and R^{8a} are each independently C₁-C₁₀ alkyl; or, the 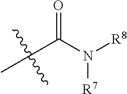
is 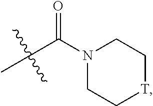
wherein the T is a covalent bond or CR⁹R¹⁰, and the R⁹ and R¹⁰ are each independently selected from the group consisting of hydrogen, deuterium, tritium, nitro, hydroxyl, mercapto, halogen, cyano, —O, imino, an amine group, an ester group, an aldehyde group, carboxyl, amido, C₁-C₆ alkyl, C₁-C₆ haloalkyl, C₁-C₆ alkoxy, and C₁-C₆ haloalkoxy.

3. The compound, the stereoisomer thereof, the tautomer thereof or the mixture form thereof, or the pharmaceutically acceptable salt thereof, or the solvate thereof, or the prodrug thereof according to claim 1, wherein the Y and Z are N, and the X is C; or
the X and Z are N, and the Y is C; or
the Z is N, and the X and Y are C; or
the Z, X, and Y are all N; or
the Z is CH, the X is N, and Y is C; or
the Z is selected from CH, the X is C, and Y is N; or
the Z is CH, and both the X and Y are N.

4. The compound, the stereoisomer thereof, the tautomer thereof or the mixture form thereof, or the pharmaceutically acceptable salt thereof, or the solvate thereof, or the prodrug thereof according to claim 1, wherein the 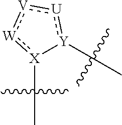
is selected from the group consisting of: 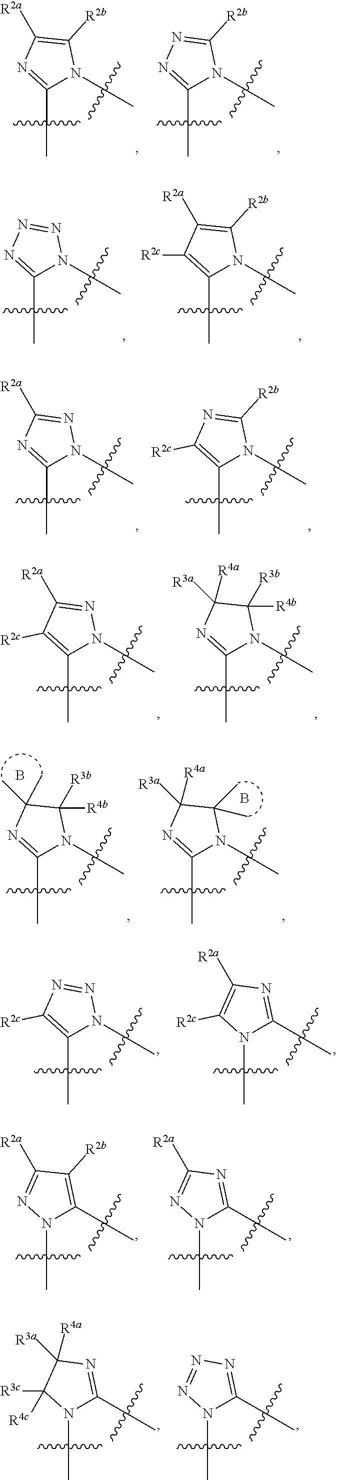 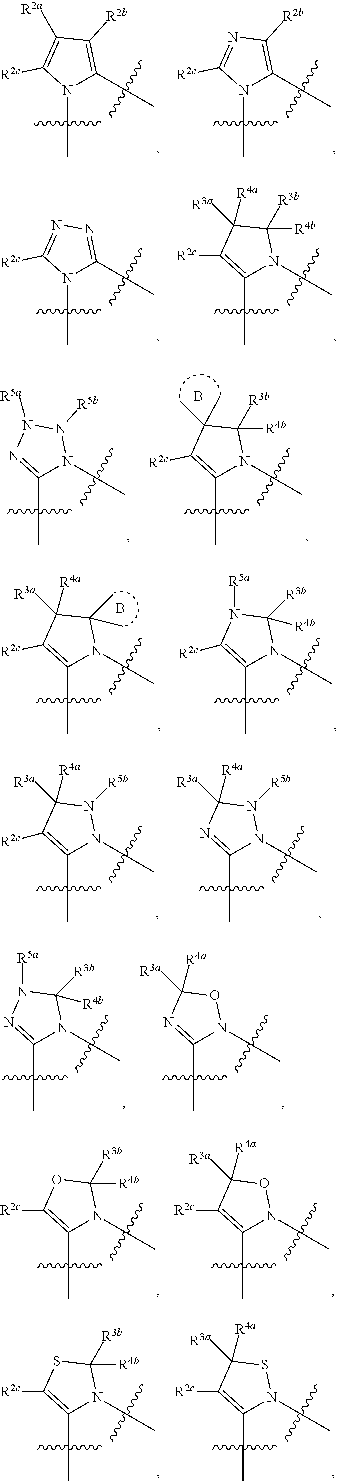 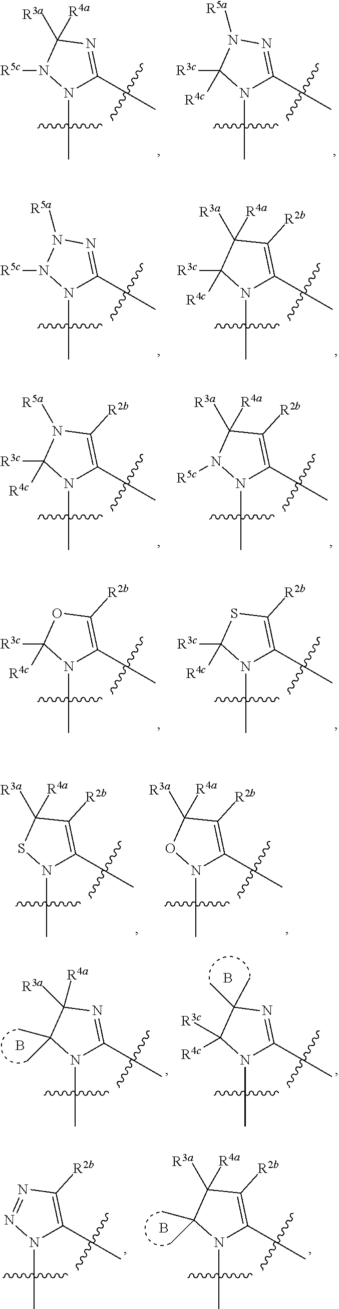 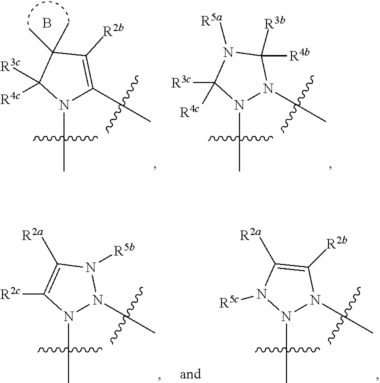
wherein the ring B is 3-12 membered cycloalkyl or 3-12 membered heterocycloalkyl, and the R^{2a}, R^{2b}, R^{2c}, R^{3a}, R^{3b}, R^{3c}, R^{4a}, R^{4b}, R^{4c}, R^{5a}, R^{5b}, and R^{5c} are each independently selected from the group consisting of hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl, or the R^{3a} and R^{4a}, the R^{3b} and R^{4b}, and the R^{3c} and R^{4c}, respectively, together form ═O or imino, and the ring B, R^{2a}, R^{2b}, R^{2c}, R^{3a}, R^{3b}, R^{3c}, R^{4a}, R^{4b}, R^{4c}, R^{5a}, R^{5b}, and R^{5c} are optionally substituted by one or two or more independently selected from the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl; or, the 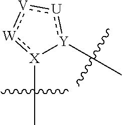
is selected from the group consisting of: 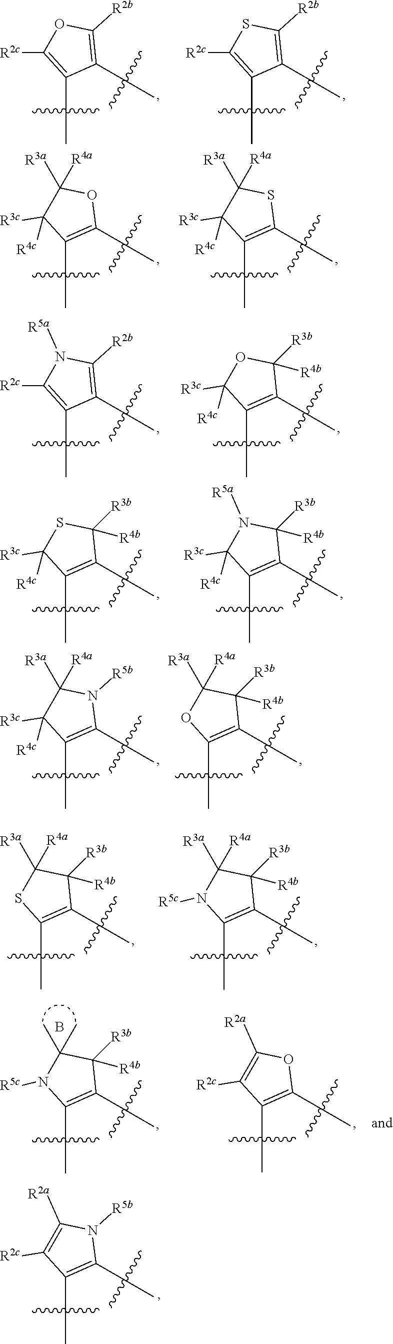
wherein the ring B is 3-12 membered cycloalkyl or 3-12 membered heterocycloalkyl, and the R^{2a}, R^{2b}, R^{2c}, R^{3a}, R^{3b}, R^{3c}, R^{4a}, R^{4b}, R^{4c}, R^{5a}, R^{5b}, and R^{5c} are each independently selected from the group consisting of hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl, or the R^{3a} and R^{4a}, the R^{3b} and R^{4b}, and the R^{3c} and R^{4c}, respectively, together form ═O or imino, and the ring B, R^{2a}, R^{2b}, R^{2c}, R^{3a}, R^{3b}, R^{3c}, R^{4a}, R^{4b}, R^{4c}, R^{5a}, R^{5b}, and R^{5c} are optionally substituted by one or two or more independently selected from the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl.

5. The compound, the stereoisomer thereof, the tautomer thereof or the mixture form thereof, or the pharmaceutically acceptable salt thereof, or the solvate thereof, or the prodrug thereof according to claim 1, wherein the compound is a compound represented by formula (II), formula (III), or formula (IV), 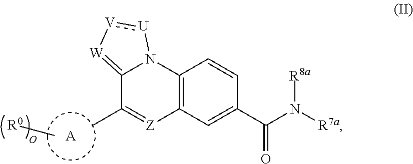
W is independently N or CR², U and V are each independently selected from N, CR², CR³R⁴, or NR⁵;
or 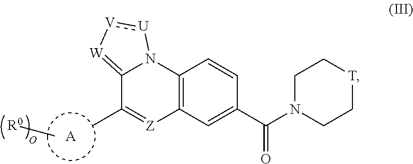
T is a covalent bond or CR⁹R¹⁰, and the R⁹ and R¹⁰ are each independently selected from the group consisting of hydrogen, deuterium, tritium, nitro, hydroxyl, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, and C₁-C₆ haloalkoxy; W is independently N or CR², and U and V are each independently N, CR², CR³R⁴, or NR⁵; or 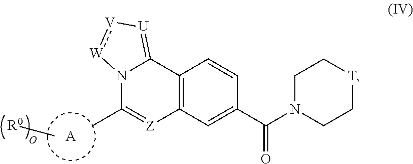
T is a covalent bond or CR⁹R¹⁰, and the R⁹ and R¹⁰ are each independently selected from the group consisting of hydrogen, deuterium, tritium, nitro, hydroxyl, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, and C₁-C₆ haloalkoxy; U is independently N or CR², and V and W are each independently N, CR², CR³R⁴, or NR⁵.

6. The compound, the stereoisomer thereof, the tautomer thereof or the mixture form thereof, or the pharmaceutically acceptable salt thereof, or the solvate thereof, or the prodrug thereof according to claim 5, wherein the 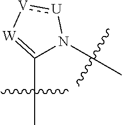
is selected from the group consisting of 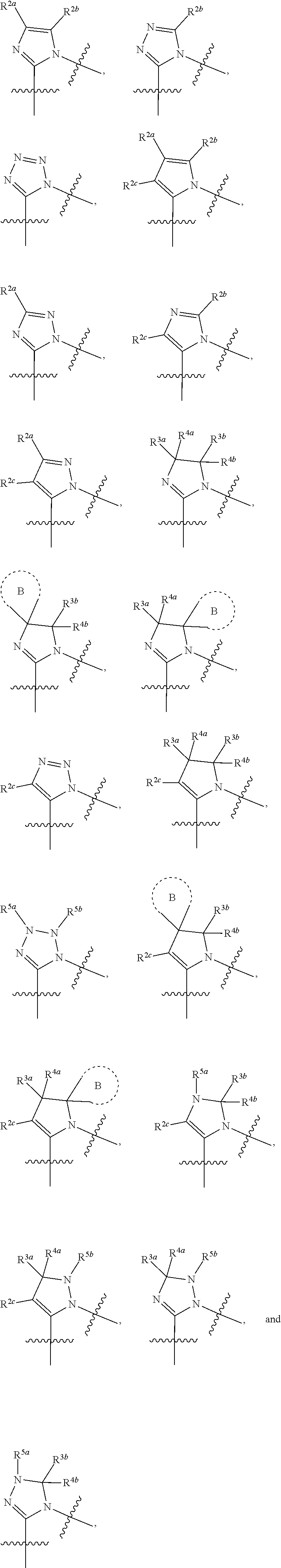
wherein the ring B is 3-12 membered cycloalkyl or 3-12 membered heterocycloalkyl;
the R^{2a}, R^{2b}, R^{2c}, R^{3a}, R^{3b}, R^{4a}, R^{4b}, R^{5a}, and R^{5b} are each independently selected from the group consisting of hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl, or the R^{3a} and R^{4a}, the R^{3b} and R^{4b}, respectively, together form ═O or imino;
wherein the ring B, R^{2a}, R^{2b}, R^{2c}, R^{3a}, R^{3b}, R^{4a}, R^{4b}, R^{5a}, and R^{5b} are optionally substituted by one or two or more independently selected from the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl.

7. The compound, the stereoisomer thereof, the tautomer thereof or the mixture form thereof, or the pharmaceutically acceptable salt thereof, or the solvate thereof, or the prodrug thereof according to claim 1, wherein the compound is a compound represented by formula (II-a) or formula (III-a) to formula (III-i),

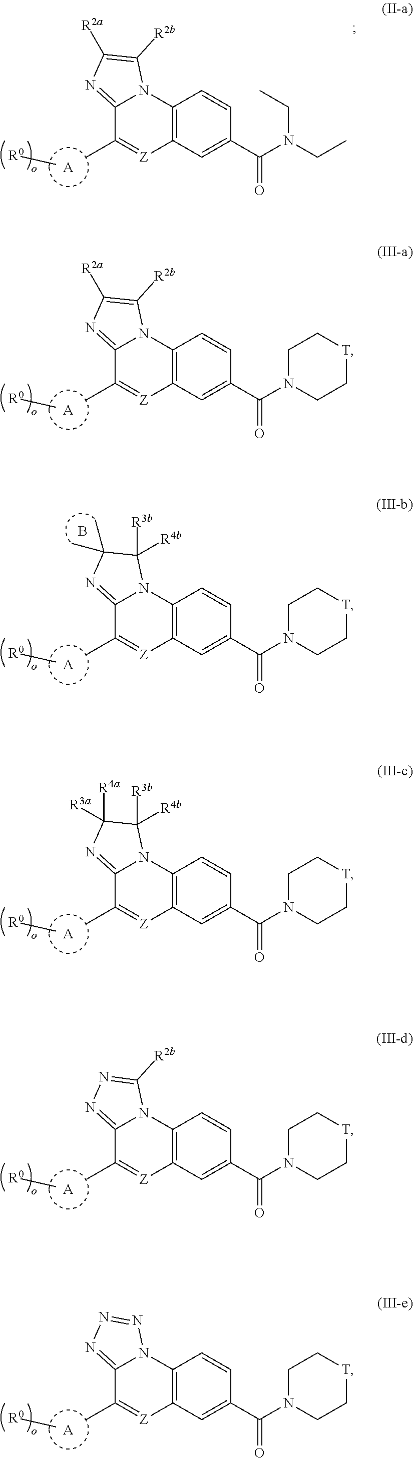

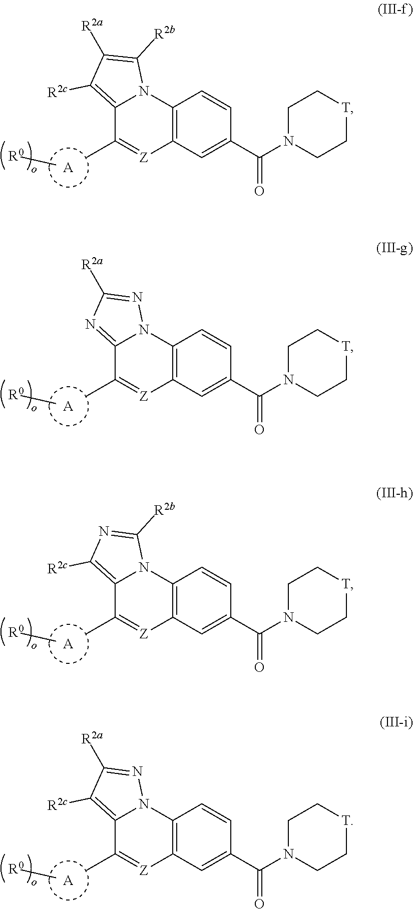

8. The compound, the stereoisomer thereof, the tautomer thereof or the mixture form thereof, or the pharmaceutically acceptable salt thereof, or the solvate thereof, or the prodrug thereof according to claim 5, wherein the T is CH₂, or CHF; or
the 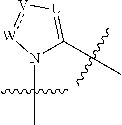
is selected from the group consisting of 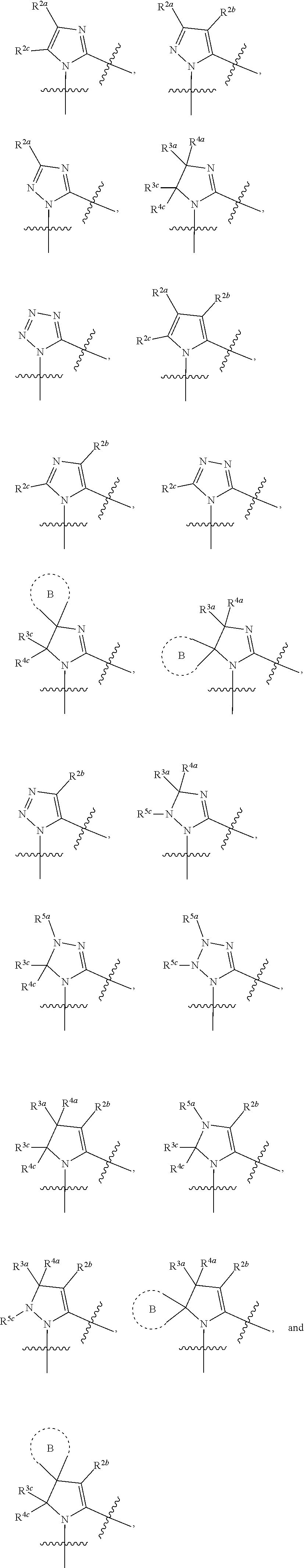
wherein the ring B is 3-12 membered cycloalkyl or 3-12 membered heterocycloalkyl;
R^{2a}, R^{2b}, R^{2c}, R^{3a}, R^{3c}, R^{4a}, R^{4c}, R^{5a}, and R^{5c} are each independently selected from the group consisting of hydrogen, deuterium, tritium, nitro, hydroxyl, mercapto, halogen, cyano, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl, or the R^{3a} and R^{4a}, the R^{3c} and R^{4c}, respectively, together form ═O or imino; wherein the ring B, R^{2a}, R^{2b}, R^{2c}, R^{3a}, R^{3c}, R^{4a}, R^{4c}, R^{5a}, and R^{5c} are optionally substituted by one or two or more independently selected from the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, 3-8 membered cycloalkyl, and 3-8 membered heterocycloalkyl.

9. The compound, the stereoisomer thereof, the tautomer thereof or the mixture form thereof, or the pharmaceutically acceptable salt thereof, or the solvate thereof, or the prodrug thereof according to claim 8, wherein the compound is any one of the structures represented by formula (IV-a) to formula (IV-h),

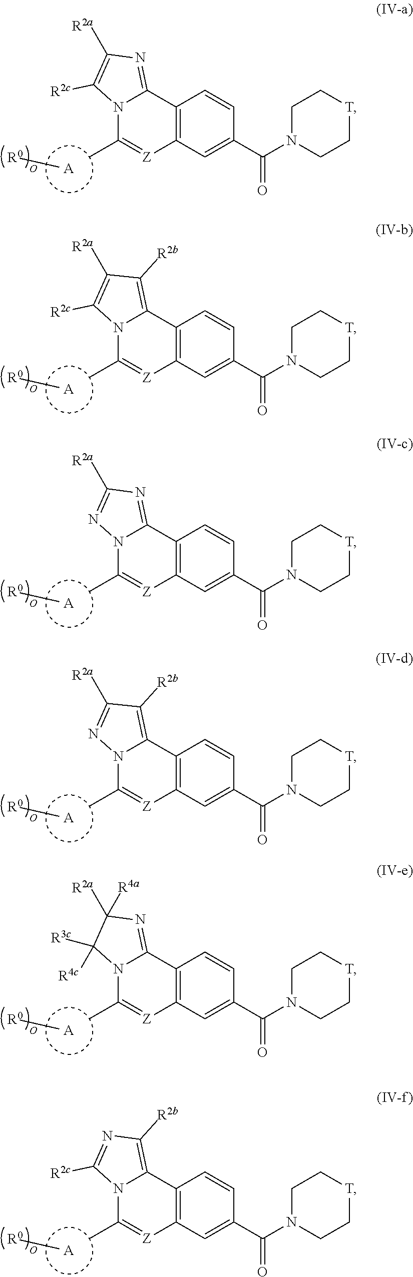

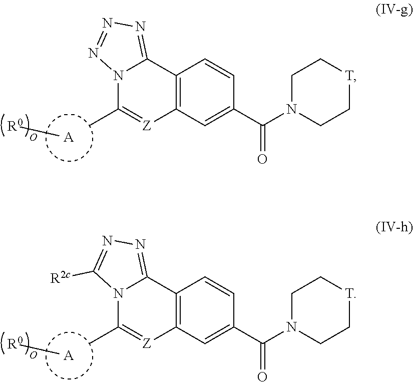

10. The compound, the stereoisomer thereof, the tautomer thereof or the mixture form thereof, or the pharmaceutically acceptable salt thereof, or the solvate thereof, or the prodrug thereof according to claim 1, wherein the ring A is selected from the group consisting of a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle; the aromatic ring and the aromatic heterocycle are monocyclic or bicyclic, the unsaturated aliphatic heterocycle is monocyclic, and the aromatic heterocycle and the unsaturated aliphatic heterocycle each independently comprise 1-3 heteroatoms, which are independently N, O, or S; or
the ring A is selected from the group consisting of benzene ring, naphthalene ring, a 5-10 membered aromatic heterocycle comprising 1-3 heteroatoms, a 6 membered unsaturated aliphatic heterocycle comprising 1-3 heteroatoms, or an 8-14 membered or an 8-12 membered fused ring consisting of benzene ring and an unsaturated aliphatic heterocycle comprising 1-3 heteroatoms, which are independently N, O, or S;
or, the ring A is selected from the group consisting of benzene ring, naphthalene ring, a 5-10 membered aromatic heterocycle comprising 1-3 heteroatoms, a 6-membered unsaturated aliphatic heterocycle comprising 1-2 heteroatoms, and an 8-14 membered or an 8-12 membered fused ring consisting of benzene ring and an unsaturated aliphatic heterocycle comprising 1-2 heteroatoms, which are independently N, O, or S.

11. The compound, the stereoisomer thereof, the tautomer thereof or the mixture form thereof, or the pharmaceutically acceptable salt thereof, or the solvate thereof, or the prodrug thereof according to claim 1, wherein the R⁰ is each independently selected from the group consisting of deuterium, tritium, nitro, hydroxyl, mercapto, fluoro, chloro, bromo, iodo, cyano, ═O, ═NH, NHCH₃, —CH₂CH₃, ═NCH(CH₃)₂, —NH₂, —NHCH₃, —N(CH₃)₂, —NHCH₂CH₃, —N(CH₂CH₃)₂, —C(O)OCH₃, —C(O)OCH₂CH₃, —C(O)O(CH₂)₂CH₃, —C(O)OCH(CH₃)₂, —OC(O)CH₃, —OC(O)CH₂CH₃, —OC(O) (CH₂)₂CH₃, —OC(O)CH(CH₃)₂, an aldehyde group, carboxyl, —COCH₃, —COCH₂CH₃, —CO(CH₂)₂CH₃, —COCH(CH₃)₂, —CONH₂, —CONHCH₃, —CON(CH₃)₂, —NHCOH, —NHCOCH₃, —N(CH₃)COCH₃, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, trifluoromethoxy, trifluoroethoxy, trichloromethoxy, trichloroethoxy, piperazinyl, N-methylpiperazinyl, morpholinyl, piperidinyl, thiomorpholinyl, oxazolidinyl, isoxazolidinyl, thiazolidinyl, isothiazolidinyl, cyclopropoxy, cyclopropylmethoxy, cyclopropyl, cyclobutyl, cyclopentyl, and cyclohexyl; or
the Z is N, the o is 0, 1, or 2, the R⁰ is each independently selected from the group consisting of deuterium, tritium, nitro, hydroxyl, mercapto, halogen, cyano, ═O, imino, an amine group, an ester group, an aldehyde group, carboxyl, carbonyl, amido, C₁-C₆ alkyl, C₁-C₆ haloalkyl, C₁-C₆ alkoxy, and C₁-C₆ haloalkoxy, the ring A is selected from the group consisting of benzene ring, naphthalene ring, a 5-10 membered aromatic heterocycle comprising 1-3 heteroatoms, a 6 membered unsaturated aliphatic heterocycle comprising 1 heteroatom, and an 8-14 membered fused ring consisting of benzene ring and an unsaturated aliphatic heterocycle comprising 1-2 heteroatoms, which are independently N, O, or S.

12. The compound, the stereoisomer thereof, the tautomer thereof or the mixture form thereof, or the pharmaceutically acceptable salt thereof, or the solvate thereof, or the prodrug thereof according to claim 1, wherein ring A is 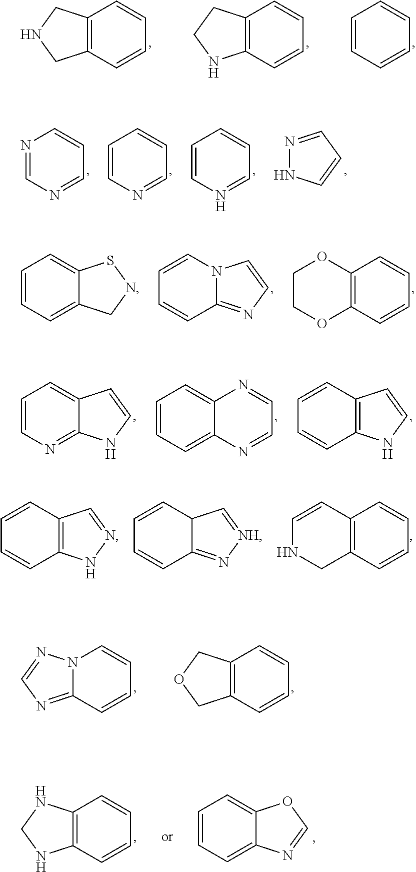
o is 0, 1, or 2,
R⁰ is each independently selected from the group consisting of deuterium, tritium, imino, an amine group, C₁-C₆ alkyl, ═O, —C(O)OCH₃, —C(O)OCH₂CH₃, —C(O)O(CH₂)₂CH₃, —C(O)OCH(CH₃)₂, carboxyl, —NH₂, —NHCH₃, —N(CH₃)₂, —NHCH₂CH₃, —N(CH₂CH₃)₂, and cyano, 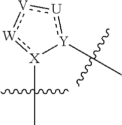
is selected from the group consisting of 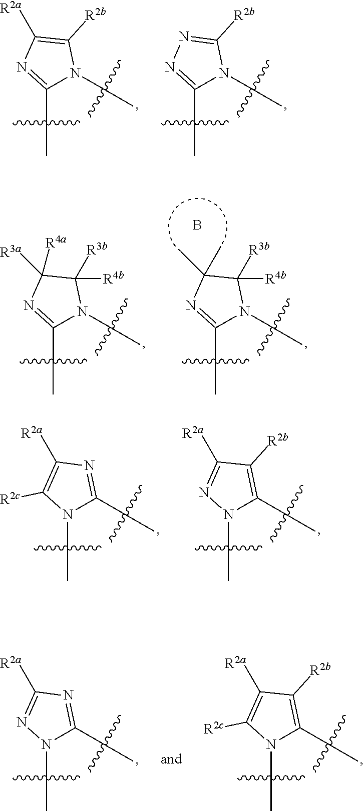
the ring B is cyclopropyl or cyclobutyl, and the R^{2a}, R^{2b}, R^{2c}, R^{3a}, R^{3b}, R^{4a}, and R^{4b} are each independently selected from the group consisting of hydrogen, deuterium, tritium, methyl, ethyl, n-propyl, isopropyl, n-butyl, isobutyl, tert-butyl, trifluoromethyl, trifluoroethyl, trichloromethyl, trichloroethyl, and cyclopropyl,
◯ represents an aromatic ring, Z is N,
R⁷ and R⁸ are each independently C₁-C₆ alkyl, or R⁷ and R⁸, together with the nitrogen atom to
which they are attached, form

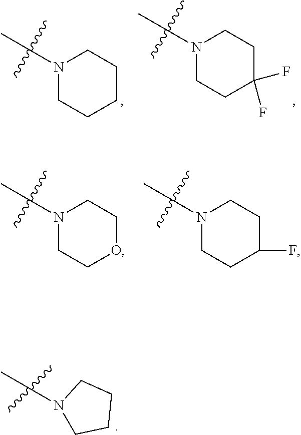 [SMILES: *N1CCCCC1 ; *N1CCC(F)(F)CC1 ; *N1CCOCC1 ; *N1CCC(F)CC1 ; *N1CCCC1]

13. The compound, the stereoisomer thereof, the tautomer thereof or the mixture form thereof, or the pharmaceutically acceptable salt thereof, or the solvate thereof, or the prodrug thereof according to claim 1, wherein the compound is selected from the group consisting of:

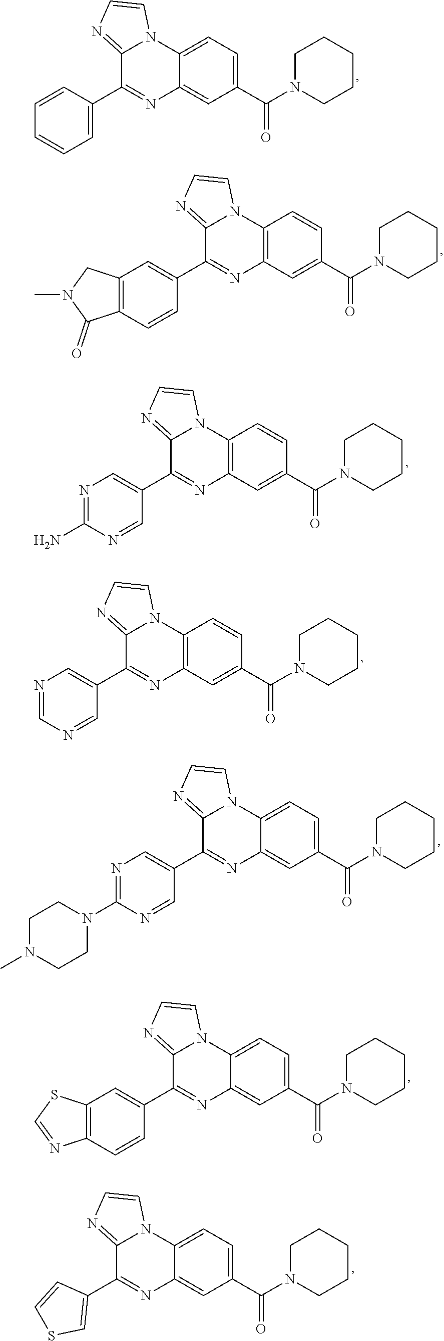

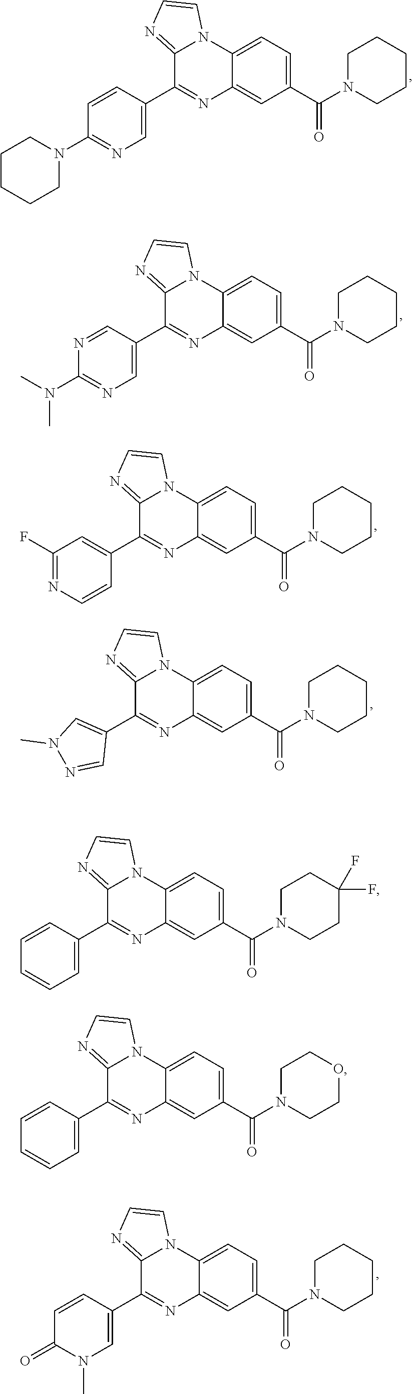

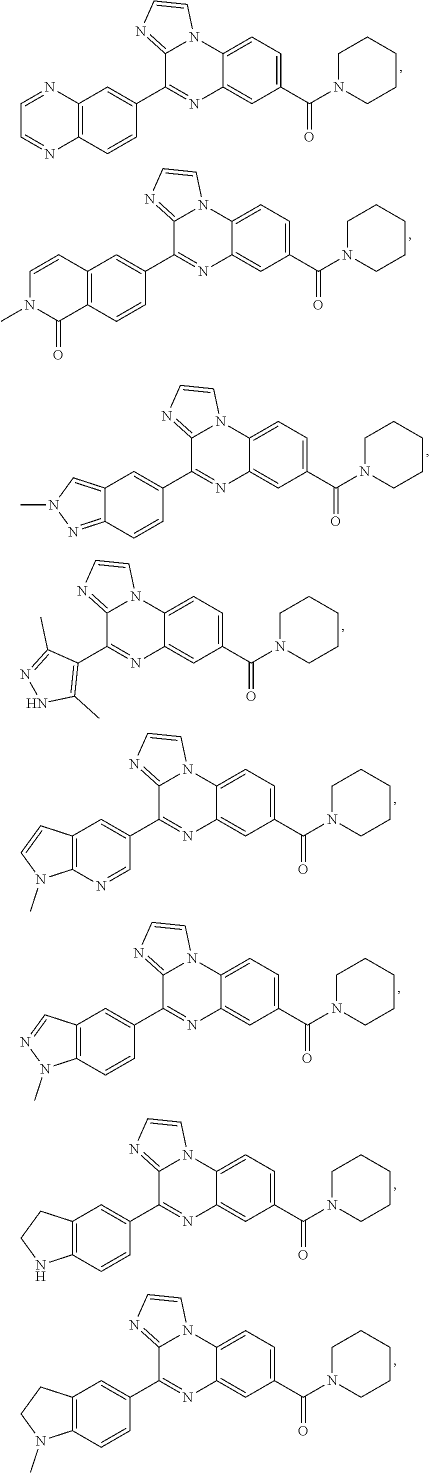

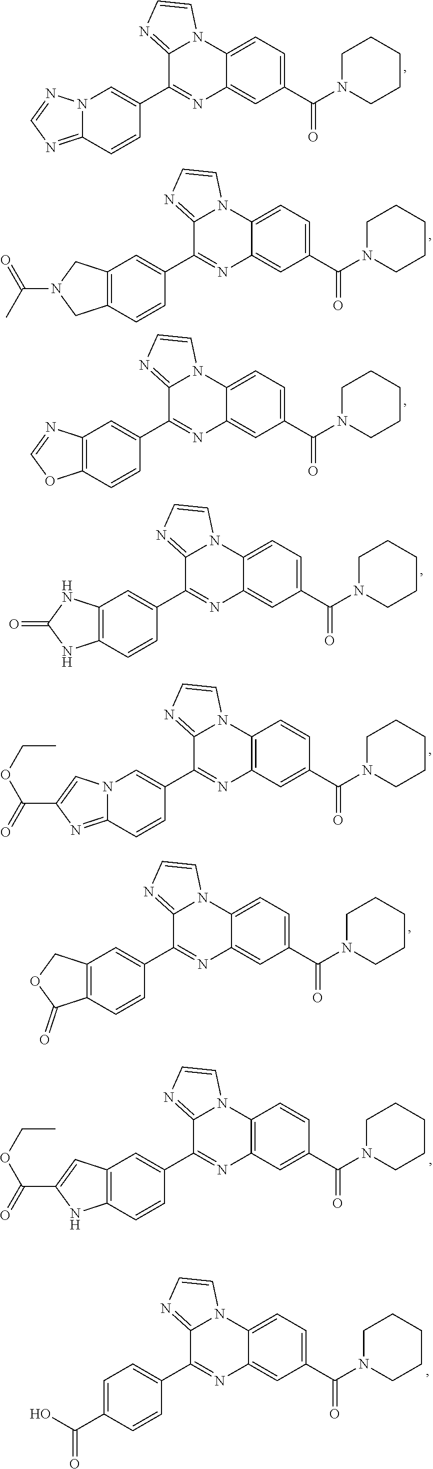

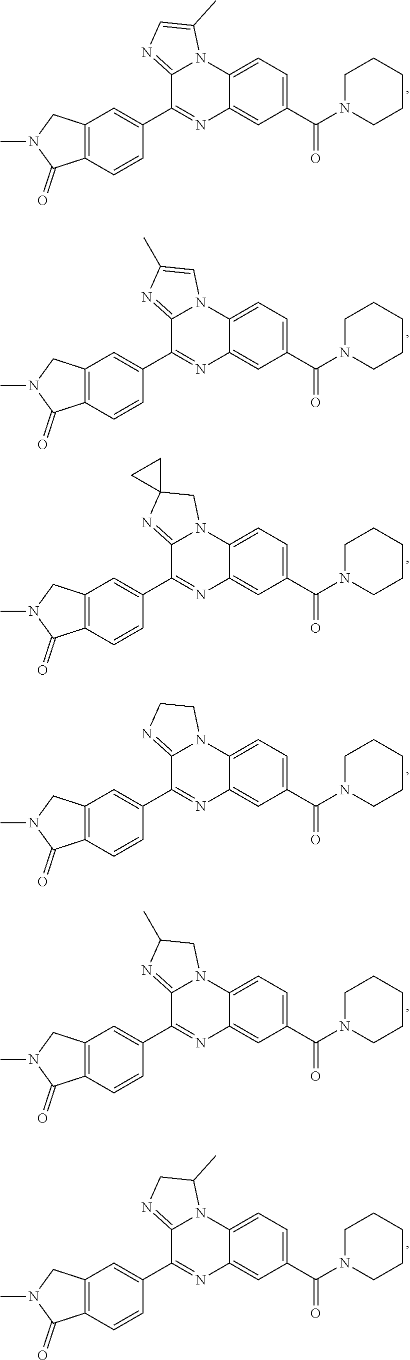 [SMILES: Cc1cnc2c(-c3ccc4c(c3)CN(C)C4=O)nc3cc(C(=O)N4CCCCC4)ccc3n12 ; Cc1cn2c(n1)c(-c1ccc3c(c1)CN(C)C3=O)nc1cc(C(=O)N3CCCCC3)ccc12 ; CN1Cc2cc(C3=Nc4cc(C(=O)N5CCCCC5)ccc4N4CC5(CC5)N=C34)ccc2C1=O ; CN1Cc2cc(C3=Nc4cc(C(=O)N5CCCCC5)ccc4N4CCN=C34)ccc2C1=O ; CC1CN2C(=N1)C(c1ccc3c(c1)CN(C)C3=O)=Nc1cc(C(=O)N3CCCCC3)ccc12 ; CC1CN=C2C(c3ccc4c(c3)CN(C)C4=O)=Nc3cc(C(=O)N4CCCCC4)ccc3N21]

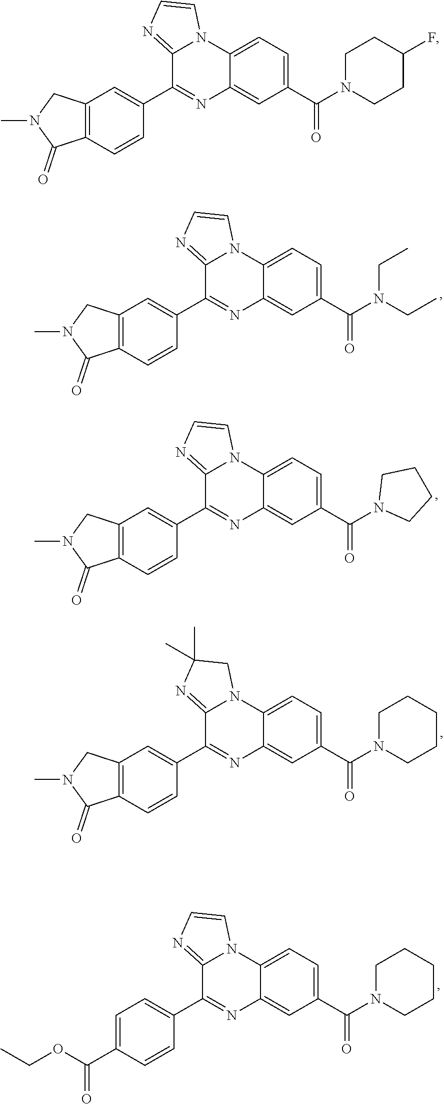

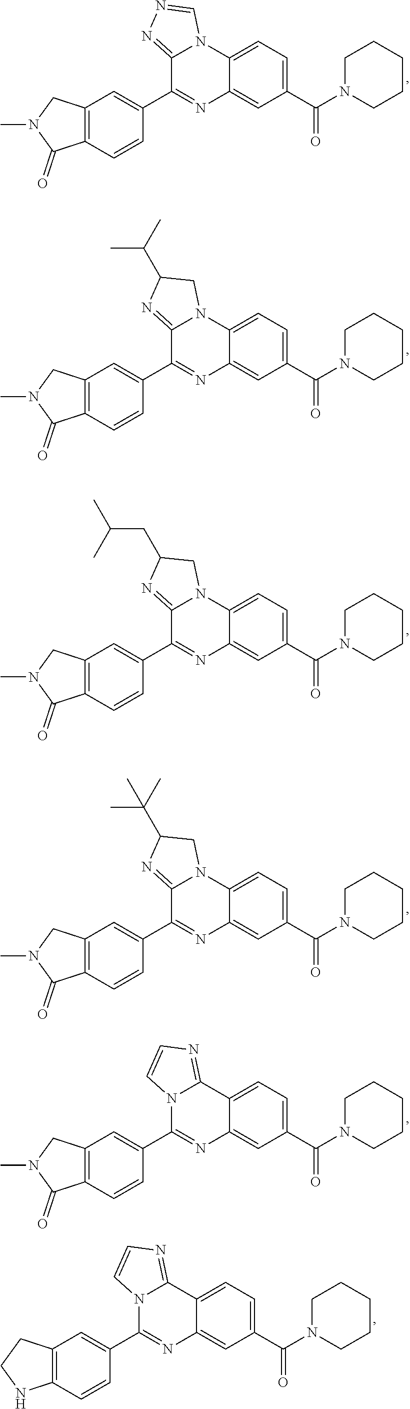 [SMILES: CN1Cc2cc(-c3nc4cc(C(=O)N5CCCCC5)ccc4n4cnnc34)ccc2C1=O ; CC(C)C1CN2C(=N1)C(c1ccc3c(c1)CN(C)C3=O)=Nc1cc(C(=O)N3CCCCC3)ccc12 ; CC(C)CC1CN2C(=N1)C(c1ccc3c(c1)CN(C)C3=O)=Nc1cc(C(=O)N3CCCCC3)ccc12 ; CN1Cc2cc(C3=Nc4cc(C(=O)N5CCCCC5)ccc4N4CC(C(C)(C)C)N=C34)ccc2C1=O ; CN1Cc2cc(-c3nc4cc(C(=O)N5CCCCC5)ccc4c4nccn34)ccc2C1=O ; O=C(c1ccc2c(c1)nc(-c1ccc3c(c1)CCN3)n1ccnc21)N1CCCCC1]

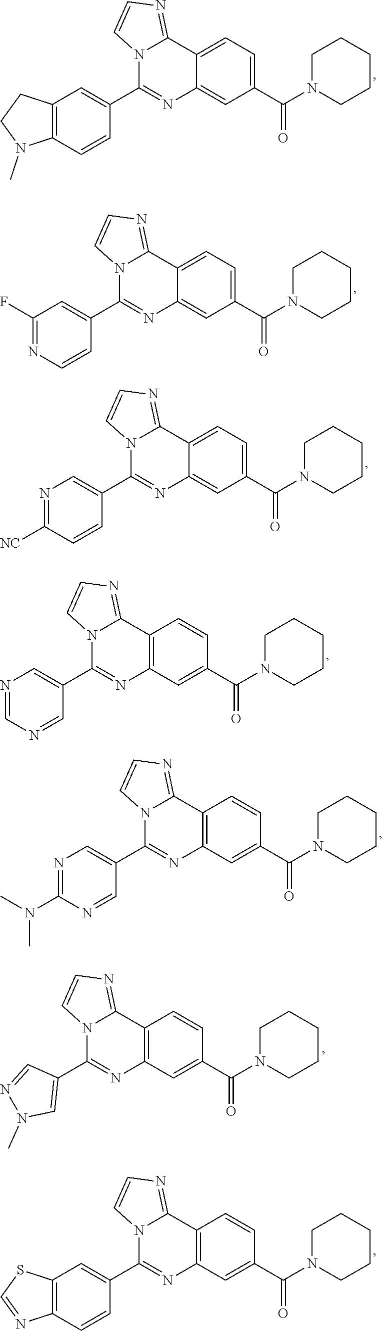

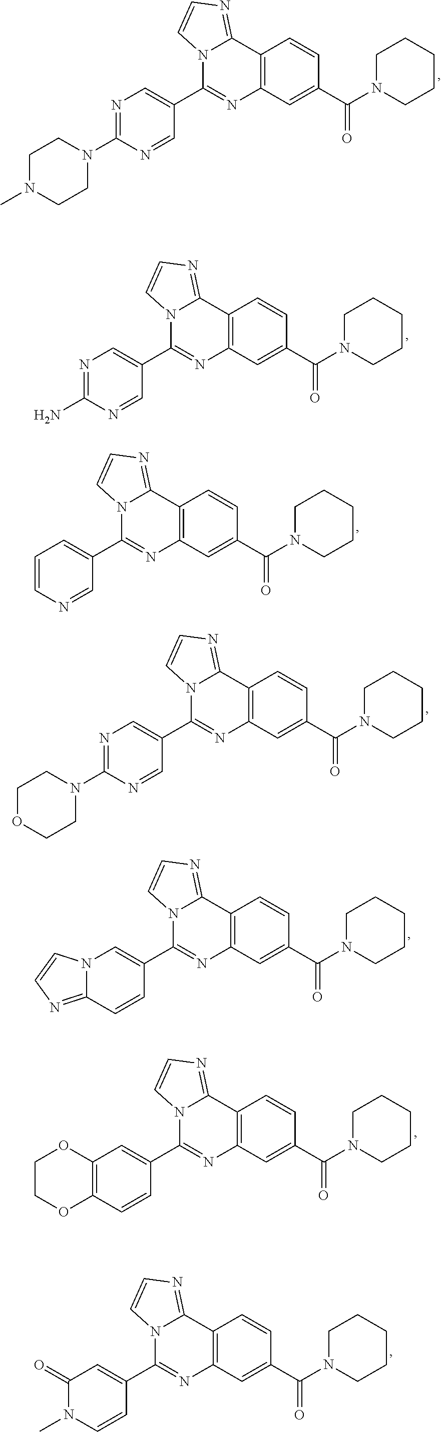

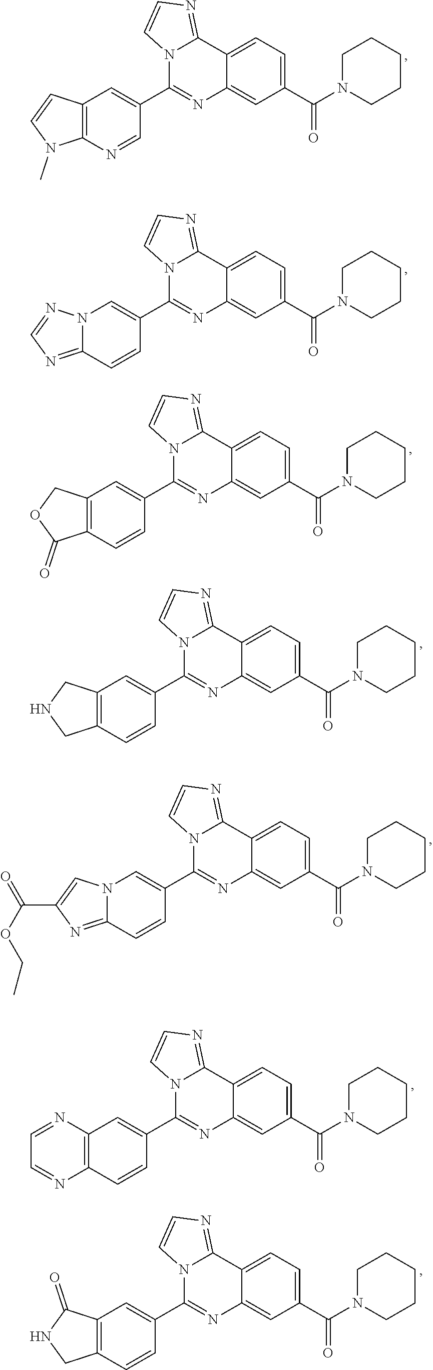

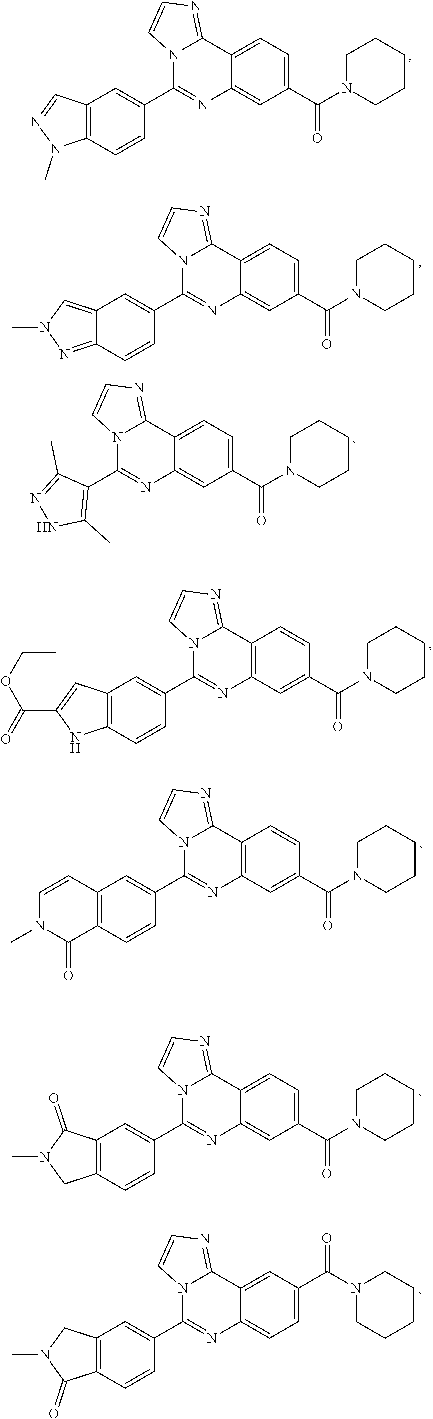

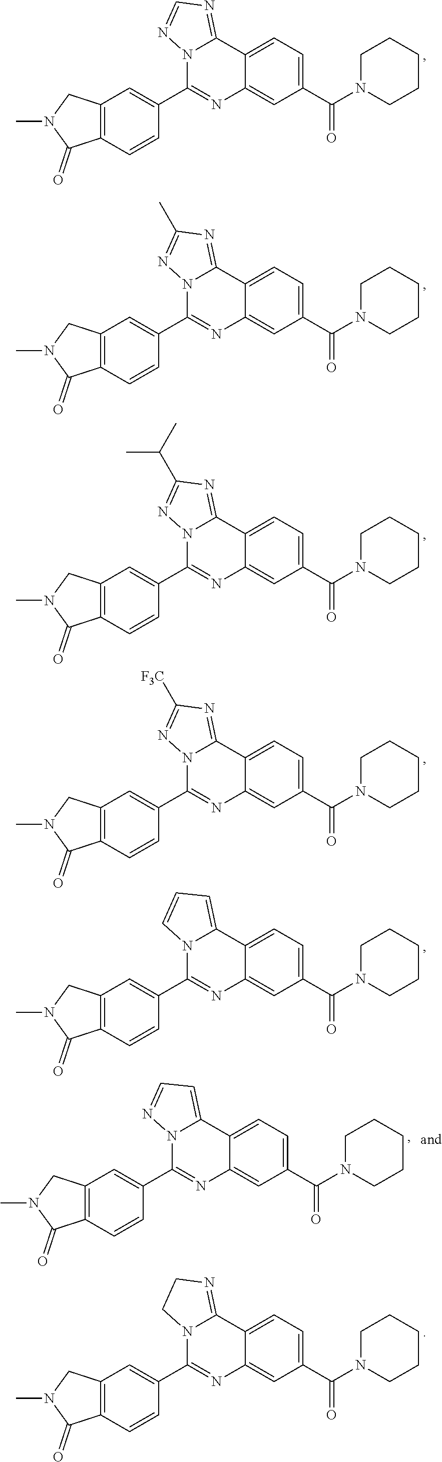

14. A pharmaceutical composition, comprising at least one of the compound,
the stereoisomer thereof, the tautomer thereof or the mixture form thereof, or the pharmaceutically acceptable salt thereof, or the solvate thereof, or the prodrug thereof according to claim 1, and at least one pharmaceutically acceptable excipient.

15. (canceled)

16. The compound, the stereoisomer thereof, the tautomer thereof or the mixture form thereof, or the pharmaceutically acceptable salt thereof, or the solvate thereof, or the prodrug thereof according to claim 2, wherein the R^{7a} and R^{8a} are each independently selected from the group consisting of methyl, ethyl, n-propyl, isopropyl, n-butyl, isobutyl, and tert-butyl; or
the T is a covalent bond or CR⁹R¹⁰, and the R⁹ and R¹⁰ are each independently selected from the group consisting of hydrogen, deuterium, tritium, nitro, cyano, —O, —NH₂, —OH, —SH, fluoro, chloro, bromo, iodo, methyl, ethyl, n-propyl, isopropyl, n-butyl, isobutyl, tert-butyl, methoxy, ethoxy, n-propoxy, isopropoxy, trifluoromethyl, trifluoroethyl, trichloromethyl, trichloroethyl, trifluoromethoxy, trifluoroethoxy, trichloromethoxy, and trichloroethoxy.

17. The compound, the stereoisomer thereof, the tautomer thereof or the mixture form thereof, or the pharmaceutically acceptable salt thereof, or the solvate thereof, or the prodrug thereof according to claim 5, wherein the R^{7a} and R^{8a} are ethyl; or
the T is a covalent bond or CR⁹R¹⁰, and the R⁰ and R¹⁰ are each independently selected from hydrogen, deuterium, tritium, nitro, cyano, —O, —NH₂, —OH, —SH, fluoro, chloro, bromo, iodo, methyl, ethyl, n-propyl, isopropyl, n-butyl, isobutyl, tert-butyl, methoxy, ethoxy, n-propoxy, isopropoxy, trifluoromethyl, trifluoroethyl, trichloromethyl, and trichloroethyl.

18. The compound, the stereoisomer thereof, the tautomer thereof or the mixture form thereof, or the pharmaceutically acceptable salt thereof, or the solvate thereof, or the prodrug thereof according to claim 6, wherein the ring B is selected from the group consisting of cyclopropyl, cyclobutyl, cyclopentyl, 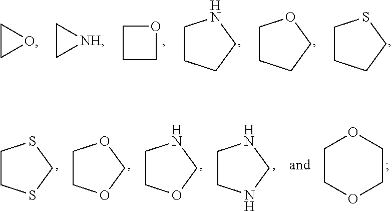
or
the R^{2a}, R^{2b}, R^{2c}, R^{3a}, R^{3b}, R^{4a}, R^{4b}, R^{5a}, and R^{5b} are each independently selected from the group consisting of hydrogen, deuterium, tritium, nitro, hydroxyl, mercapto, fluoro, chloro, bromo, iodo, cyano, —NH₂, carboxyl, —CONH₂, —NHCOOH, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl, or the R^{3a} and R^{4a}, the R^{3b} and R^{4b}, respectively, together form ═O, ═NH.

19. The compound, the stereoisomer thereof, the tautomer thereof or the mixture form thereof, or the pharmaceutically acceptable salt thereof, or the solvate thereof, or the prodrug thereof according to claim 10, wherein the ring A is selected from the group consisting of

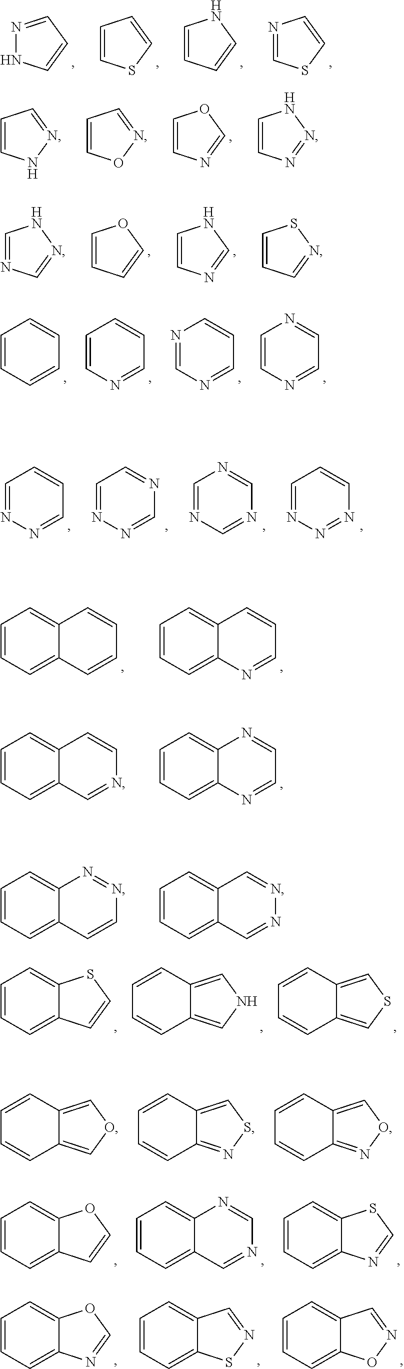

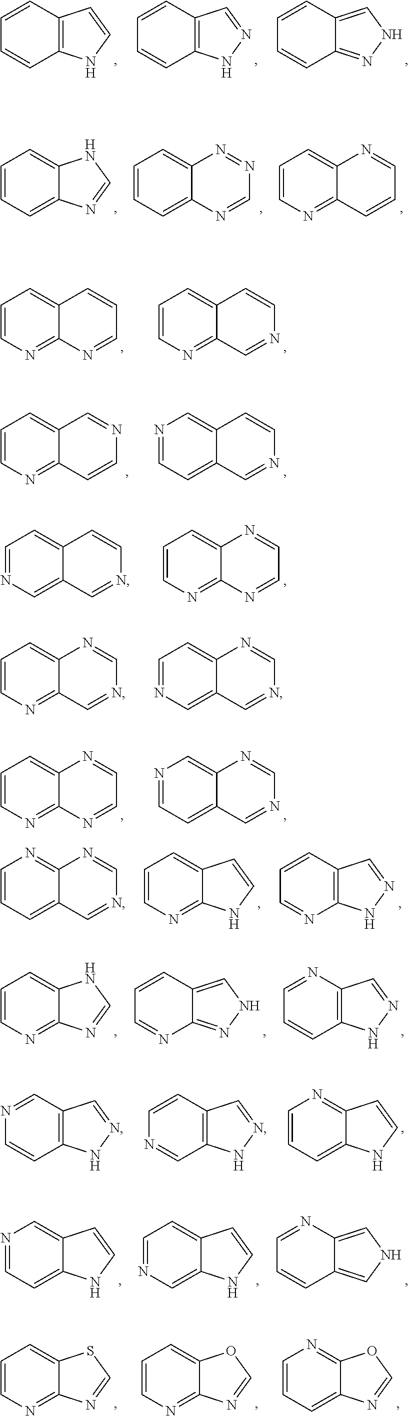

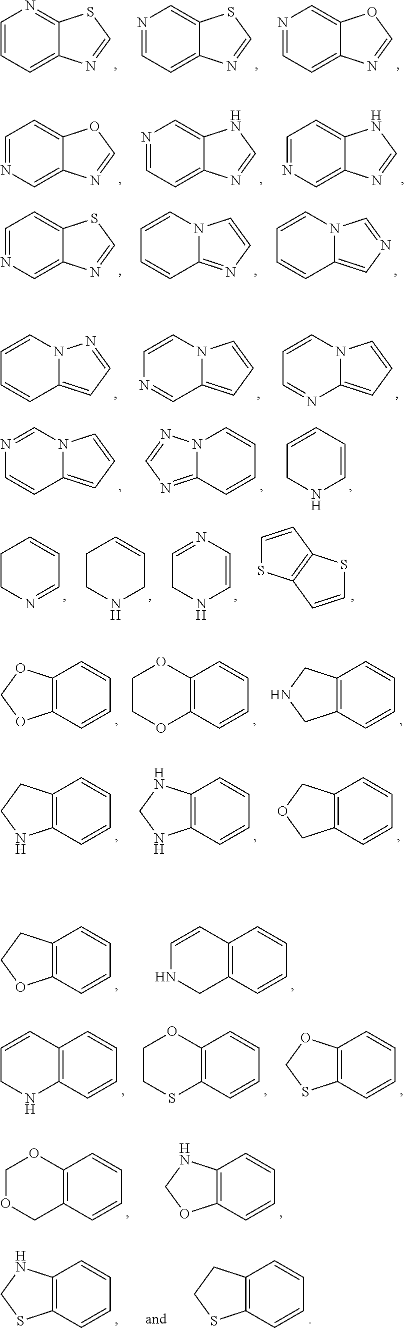

20. A method of treating or preventing a disease associated with 15-PGDH, comprising administering to a subject in need thereof the compound, the stereoisomer, the tautomer or the mixture form thereof, or the pharmaceutically acceptable salt thereof, or the solvate thereof, or the prodrug thereof according to claim 1, or a pharmaceutical composition comprising the same.

21. The method according to claim 20, wherein the method is used for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, underactive bladder; or for promoting hair growth, pigmentation, tissue repair, tissue regeneration, implant in stem cell transplantation or bone marrow transplantation or organ transplantation, neurogenesis and neuronal cell death, or muscle regeneration, and cervical ripening; or for resistance to the toxicity of chemotherapy and the toxicity of immunosuppressant.
